(19) United States
(12) Patent Application Publication
(10) Pub. No.: US 2026/0306492 A1
(43) Pub. Date: Oct. 8, 2026

(54) USE OF MILVEXIAN IN THE TREATMENT AND PREVENTION OF THROMBOTIC CONDITIONS IN PATIENTS WITH ATRIAL FIBRILATION

(71) Applicants: BRISTOL-MYERS SQUIBB COMPANY, Princeton, NJ (US); JANSSSEN PHARMACEUTICA NV, Beerse (BE)

(72) Inventors: John STRONY, Raritan, NJ (US); Gary PETERS, Raritan, NJ (US); Madhu CHINTALA, Spring House, PA (US); Christopher NESSEL, Bridgewater, NJ (US); Danshi LI, Princeton, NJ (US); Joseph LUETTGEN, Princeton, NJ (US); Puneet MOHAN, Princeton, NJ (US); Jay HORROW, Princeton, NJ (US)

(21) Appl. No.: 19/476,294

(22) PCT Filed: Apr. 19, 2024
(86) PCT No.: PCT/US2024/025401

§ 371 Date: Oct. 17, 2025

Related U.S. Application Data
(60) Provisional application No. 63/497,101, filed on Apr. 19, 2023.
(60) Provisional application No. 63/578,508, filed on Aug. 24, 2023.
(60) Provisional application No. 63/582,313, filed on Sep. 13, 2023.

(51) Int. Cl. A61K 31/513; A61K 9/20; A61K 9/28; A61P 7/02; A61P 9/10

(52) U.S. Cl. CPC A61K 31/513; A61K 9/2013; A61K 9/2018; A61K 9/2054; A61K 9/28; A61P 7/02; A61P 9/10

(57) ABSTRACT

The disclosure provides methods of using milvexian for treating or preventing thrombotic events in human patients with atrial fibrillation or flutter.

### CROSS-REFERENCE TO RELATED APPLICATIONS

This application claims the benefit of U.S. Provisional Application No. 63/497,101, filed Apr. 19, 2023, U.S. Provisional Application No. 63/578,508, filed Aug. 24, 2023, and U.S. Provisional Application No. 63/582,313, filed Sep. 13, 2023, each of which is incorporated by reference herein in its entirety.

### TECHNICAL FIELD

The disclosure pertains to the use of milvexian for treating or preventing major thrombotic conditions with less bleeding than the current standard-of-care in human patients with atrial fibrillation, including those for whom Factor Xa (FXa) inhibitors are underutilized or not indicated.

### BACKGROUND

Atrial fibrillation (AF) affects about 2.3 million people in North America and 4.5 million people in the European Union and is emerging as a growing public health concern because of the aging of the population. AF without significant valve disease (commonly referred to as non-valvular AF) is the most common cardiac arrhythmia worldwide and is a major burden to healthcare services.

AF is a condition in which the upper chambers of the heart beat in an uncoordinated and disorganized fashion, resulting in a very irregular and fast rhythm (i.e., an irregular heartbeat). When blood is not completely pumped out of the heart's chambers, it can pool and clot. Sometimes these blood clots will travel through the blood and block off the blood supply in different areas of the body which causes thrombotic events (e.g., primarily embolism from the left atrial appendage, see FIG. 2). For example, a stroke occurs when a blood clot blocks off blood flow to part of the brain which can cause brain damage, disability, or even death. Incidence of ischemic stroke in patients with AF increases nearly five-fold compared to those patients without AF. Consequently, about 15 percent of strokes result from AF.

It is known that atrial fibrillation itself can cause changes in the electrical parameters of the heart known as electrical remodeling and in the structure of the cardiac chambers known as structural remodeling which tend to decrease the chances of the patient to get back into normal sinus rhythm. This vicious circle whereby “atrial fibrillation begets atrial fibrillation” has been well documented since the 1990s (Wijffels et al., Atrial fibrillation begets atrial fibrillation, A study in awake chronically instrumented goats. Circulation, 1995 Oct. 1; 92 (7): 1954-68). It explains why when patients have been in atrial fibrillation for a long time, they develop permanent atrial fibrillation with little or no chance to recover from this arrhythmia which becomes chronic.

AF is increasingly frequent with advancing age and is often caused by age-related changes in the heart, physical or psychological stress, agents that stimulate the heart, such as caffeine, or as a result of cardiovascular disease. The number is expected to double in the next 20 years. Without appropriate management, AF can lead to serious complications, such as ischemic stroke and congestive heart failure (Polimeni et al., The risk of myocardial infarction in patients with atrial fibrillation: an unresolved issue. Intern Emerg Med. 2010; 5:91-94; Virani, Heart disease and stroke statistics 2021 update, Circulation 2021; 143: doi: 10.1161/CIR.0000000000000950, Section on AF is pages e311-e328). The presence of multiple comorbidities in those who experience AF highlights the need for a more comprehensive treatment strategy that provides benefit across a broad spectrum of clinical events associated with atherothrombosis and systemic embolism.

Milvexian (BMS-986177/JNJ-70033093) is a direct-acting, high-affinity inhibitor of human coagulation FXIa (Dilger et al. Discovery of milvexian, a high-affinity, orally bioavailable inhibitor of factor XIa in clinical studies for antithrombotic therapy. J Med Chem 2022; 65 (3): 1770-85). Milvexian is a macrocyclic compound having the structure of Formula (I):

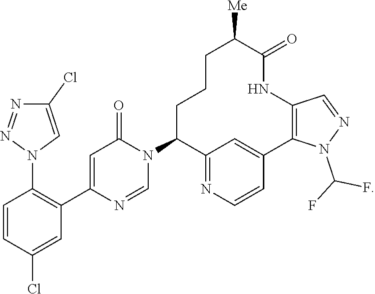 [SMILES: C[C@@H]1CCC[C@H](n2cnc(-c3cc(Cl)ccc3-n3cc(Cl)nn3)cc2=O)c2cc(ccn2)-c2c(cnn2C(F)F)NC1=O]

Milvexian is also known by its chemical name (5R,9S)-9-(4-(5-chloro-2-(4-chloro-1H-1,2,3-triazol-1-yl)phenyl)-6-oxopyrimidin-1 (6H)-yl)-2¹-(difluoromethyl)-5-methyl-2¹H-3-aza-1 (4,2)-pyridina-2 (5,4)-pyrazolacyclonaphan-4-one.

Milvexian and a method of preparing milvexian are described in U.S. Pat. No. 9,453,018, which is hereby incorporated by reference in its entirety. Solvates, crystalline forms, and amorphous forms of milvexian have been described in WO2021207659 and WO2022081473. An amorphous solid dispersion composition of milvexian in one or more polymers has been described in WO2020210629, which is hereby incorporated by reference in its entirety.

### SUMMARY

The methods of the disclosure provide a novel treatment regimen comprising a novel immediate release oral tablet comprising milvexian for anticoagulation in patients with atrial fibrillation or flutter, and at increased risk of thromboembolic stroke and increased risk of bleeding. The methods of the disclosure could break through the perceived efficacy ceiling of currently available anticoagulants for patients with AF or flutter (Bentley et al. infra). The methods of the disclosure introduce an opportunity to expand the use of FXIa inhibitors as anticoagulants to areas or diseases where existing anticoagulants are underused or not used. In some embodiments of the method described herein, the novel treatment regimen may further comprise standard-of-care antiplatelet therapy (e.g., dual antiplatelet therapy (DAPT), or single antiplatelet therapy (SAPT)).

In some aspects, the disclosure provides methods for preventing one or more of stroke and non-central nervous system (CNS) systemic embolism in a patient with atrial fibrillation or flutter, wherein the method comprises administering to the human patient a pharmaceutical composition comprising about 50 mg, about 75 mg, about 87.5 mg, or about 100 mg of milvexian (or a pharmaceutically acceptable salt or solvate thereof, on a milvexian basis); wherein the composition is administered twice daily. In some aspects, the disclosure provides methods for preventing one or more of stroke and non-central nervous system (CNS) systemic embolism events in a patient with a history of atrial fibrillation or flutter, wherein the method comprises administering to the human patient a pharmaceutical composition comprising about 50 mg, or about 100 mg of milvexian (or a pharmaceutically acceptable salt or solvate thereof, on a milvexian basis); wherein the composition is administered twice daily. In some aspects, the disclosure provides methods for preventing one or more of stroke and non-central nervous system (CNS) systemic embolism events in a patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis); wherein the composition is administered twice daily.

In some aspects, the disclosure provides methods for preventing one or more of adverse cardiovascular events in a patient with atrial fibrillation or flutter, wherein each adverse cardiovascular event is selected from the group consisting of cardiovascular death, myocardial infarction, stroke, and non-central nervous system (CNS) systemic embolism, wherein the method comprises administering to the human patient a pharmaceutical composition comprising about 50 mg, about 75 mg, about 87.5 mg, or about 100 mg of milvexian (or a pharmaceutically acceptable salt or solvate thereof, on a milvexian basis); wherein the composition is administered twice daily. In some aspects, the disclosure provides methods for preventing one or more of major adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg, or about 100 mg of milvexian (or a pharmaceutically acceptable salt or solvate thereof, on a milvexian basis); wherein the composition is administered twice daily. In some aspects, the disclosure provides methods for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.

### BRIEF DESCRIPTION OF THE DRAWINGS

FIG. 1 shows coagulation pathways. Legend: FXII, factor XII; FXIIa, activated factor XII; FXI, factor XI; FXIa, activated factor XI; FIX, factor IX; FIXa, activated factor IX; FVIIa, activated factor VII; FVII, factor VII; FX, factor X; FXa, activated factor X, as described in Dilger A K, et al., Discovery of milvexian, a high-affinity, orally bioavailable inhibitor of factor XIa in clinical studies for antithrombotic therapy. J Med Chem. 2022; 65 (3): 1770-1785.

FIG. 2 illustrates the clot formation associated with atrial fibrillation and adverse vascular events, e.g. ischemic stroke and non-central nervous system (CNS) systemic embolism.

FIG. 3 illustrates median of milvexian plasma concentration (ng/ml) by nominal sampling time Day 1 through Day 14, linear scale, by treatment Groups of 25 mg, 50 mg and 200 mg QD dosing and 25 mg, 50 mg, 100 mg and 200 mg BID dosing. Subjects who received twice daily and once daily doses of milvexian from 25 mg to 200 mg exhibited increased milvexian concentration with increase in dose. For example, Day 4 predose concentration of 1,570.71 ng/mL was achieved at 100 mg twice daily dose vs 3,699.01 ng/ml at 200 mg twice daily dose. A similar increase in dose dependent concentration was observed with the once daily regimens, eg, 220.23 ng/mL at 50 mg once daily vs 1,228.63 ng/ml at 200 mg once daily, at Day 4 predose Group; PK Analysis Set (AXIOMATIC-TKR) The observed concentration on Day 4 (12 hours after dosing) was higher for 50 mg and 200 mg once daily compared to 25 mg and 100 mg twice daily, respectively; but the values were lower for once daily dosing compared to twice daily dosing at Day 7, 12 hours after dosing. On Day 4, Tₘₐₓ was approximately 2 hours after dosing for milvexian 25 mg and 100 mg twice daily vs approximately 4 hours after dosing for milvexian 50 mg and 200 mg once daily. These values were consistent with the median Tₘₐₓ of 2-to-3 hours for once daily, and approximately 3 hours twice daily observed in healthy volunteers. Mean trough plasma concentrations of milvexian achieved steady state condition approximately on Day 4 following 25 mg, 50 mg, or 100 mg doses after twice daily administration to patients as a spray-dried amorphous solid dispersion formulation in capsule (25 mg or 100 mg strength as described in WO2020210629). (AXIOMATIC-TKR Phase II). In some embodiments, milvexian has a terminal plasma half-life (T_{1/2}) ranging from about 13 hours to about 16 hours. With a terminal half-life ranging from 13-16 hours, milvexian administered twice daily can remain sufficiently active between the two administrations.

FIG. 4 illustrates median of aPTT ratio to baseline, by nominal sampling time Day 1 through Day 14 by treatment Groups of 25 mg, 50 mg and 200 mg QD dosing and 25 mg, 50 mg, 100 mg and 200 mg BID dosing. A dose related increase in aPTT was observed with milvexian. In this study, the VTE reduction observed with milvexian 50 mg twice daily was superior to enoxaparin and was associated with an approximately 2.3-fold increase in aPTT. Higher milvexian doses (eg, greater than 50 mg twice daily) that resulted in even lower VTE rates accompanied by further increases in aPTT, with the maximum 200 mg twice daily dose resulting in an approximately 3.4-fold increase in aPTT ratio. (AXIOMATIC-TKR Phase II).

FIG. 5 illustrates median of FXI clotting activity % change from baseline, by nominal sampling time Day 1 through Day 14 by treatment Groups of 25 mg, 50 mg and 200 mg QD dosing and 25 mg, 50 mg, 100 mg and 200 mg BID dosing. A dose dependent decrease in FXI clotting activity was observed across the milvexian dose range tested. The VTE reduction observed with milvexian 50 mg twice daily was superior to enoxaparin and was associated with an approximately 18% mean reduction in FXI clotting activity. At the highest dose (200 mg twice daily), an approximately 79% mean reduction in FXI clotting activity was observed. (AXIOMATIC-TKR Phase II).

FIG. 6A illustrates the highest concentration of thrombin as measured by mean peak height ratio to baseline, FIG. 6B illustrates median of endogenous thrombin potential (ETP) area under the curve ratio to baseline, and FIG. 6C illustrates median of lag time ratio to baseline in the TGA measurements, by nominal sampling time Day 1 through Day 14 by treatment Groups of 25 mg, 50 mg and 200 mg QD dosing and 25 mg, 50 mg, 100 mg and 200 mg BID dosing. A clear and robust dose dependent reduction in peak thrombin generation was observed upon milvexian treatment, mean peak height ratio to baseline was 0.18 on Day 10-to-14 for 200 mg twice daily as compared to 0.27 for 100 mg twice daily (FIG. 6A). There were dose related reductions in peak thrombin and area under the curve (total thrombin generation) (FIG. 6B), and there was a dose related prolongation in the lag time which indicates a delay in time to clot initiation (FIG. 6C). (AXIOMATIC-TKR Phase II).

FIG. 7A shows the Day 1 milvexian plasma concentration as a function of time after BID administration of a film-coated direct compression tablet (2×100 mg) of the disclosure compared to the Day 1 milvexian plasma concentration as a function of time after BID administration of a milvexian-containing capsule (2×100 mg). See Example 4.

FIG. 7B shows the Day 5 milvexian plasma concentration as a function of time after BID administration of a film-coated direct compression tablet (2×100 mg) of the disclosure compared to the Day 5 milvexian plasma concentration as a function of time after BID administration of a milvexian-containing capsule (2×100 mg). See Example 4.

FIG. 7C shows the Day 1 milvexian plasma concentration as a function of time after BID administration of a film-coated direct compression tablet (1×25 mg) of the disclosure compared to the Day 1 milvexian plasma concentration as a function of time after BID administration of a milvexian-containing capsule (1×25 mg). See Example 4.

FIG. 7D shows the Day 5 milvexian plasma concentration as a function of time after BID administration of a film-coated direct compression tablet (1×25 mg) of the disclosure compared to the Day 5 milvexian plasma concentration as a function of time after BID administration of a milvexian-containing capsule (1×25 mg). See Example 4.

FIG. 8 shows a odds ratio plot resulting from the model-based meta-analysis described in the Dose Selection section of Example 1.

FIG. 9 shows a graphical representation of the design of the study described in Example 1.

### DETAILED DESCRIPTION OF ILLUSTRATIVE EMBODIMENTS

The physiopathology of atrial fibrillation includes abnormal changes in the atrial tissue, inflammation and endothelial damages/dysfunction leading to an overexpression of tissue factor. Patients with atrial fibrillation are typically prescribed long-term anticoagulants in the form of either vitamin K antagonists or non-vitamin K antagonist oral anticoagulants; however, both carry a potential risk of adverse bleeding. The advent of direct oral anticoagulants (DOACs), that include factor Xa and direct thrombin inhibitors, has resulted in major advances in the prevention of thromboembolic events in patients with non-valvular AF (NVAF) (Granger et al., Apixaban versus warfarin in patients with atrial fibrillation, N. Engl. J. Med. 2011; 365 (11): 981-992; Patel et al., Rivaroxaban versus warfarin in nonvalvular atrial fibrillation, N Engl J Med. 2011; 365:883-891 including Supplement). These DOACs reduced mortality by approximately 10% and hemorrhagic stroke by nearly half, compared with warfarin (Liew et al., Comparing mortality in patients with atrial fibrillation who are receiving a direct-acting oral anticoagulant or warfarin: a meta-analysis of randomized trials, J Thromb Haemost. 2014; 12:1419-24; Hellenbart et al., Evaluation of bleeding in patients receiving direct oral anticoagulants, Vasc Health and Risk Manag. 2017; 13:325-342). However, these DOACs can also cause a person to have bleeding in different parts of their body (like their stomach or intestines), or make it hard to control bleeding. Bleeding risk varies among AF subpopulations. In patients receiving DOACs with single or dual antiplatelet therapy (SAPT, DAPT), the risk of bleeding is substantially increased (Hellenbart et al. supra; Ravi et al., Meta-Analysis Comparing Double Versus Triple Antithrombotic Therapy in Patients With Atrial Fibrillation and Coronary Artery Disease, Am J Cardiol. 2020; 125:19-28).

Additionally, patients who are elderly, frail, or diagnosed with severe renal insufficiency are at an increased risk of bleeding (Hindricks et al., 2020 ESC Guidelines for the diagnosis and management of atrial fibrillation, Eur Heart J. 2020; 42:373-498). Fear of the risk of bleeding restrains practitioners from prescribing, and prevents patients with AF from taking anticoagulation therapy to reduce the risk of stroke and systemic embolism (Sen, et al., Physician's Fear of Anticoagulant Therapy in Nonvalvular Atrial Fibrillation, Am J Med Sci. 2014; 348(6):513-521). Of 58,084 patients discharged from a hospital after stroke with a pre-stroke CHA2DS2-VASc score ≥2 and receiving no anticoagulation, only 34% had a reason for lack of anticoagulation: risk of bleeding topped the list at 16%, with risk of falls at 10% (Xian et al., Association of Preceding Antithrombotic Treatment With Acute Ischemic Stroke Severity and In-Hospital Outcomes Among Patients With Atrial Fibrillation, JAMA. 2017; 317 (10): 1057-1067). The undertreatment or withheld treatment of various population segments with AF (Verdino, Untreated atrial fibrillation in the United States of America: Understanding the barriers and treatment options, J Saudi Heart Assoc 2015; 27:44-49; Xian et al. supra) provide a foundation for a new treatment that prevents stroke and systemic embolism at least as well as a DOAC, but with fewer bleeding events.

An aging population results in growing numbers of patients with atrial fibrillation who also have concomitant coronary artery disease (CAD) and/or peripheral artery disease (PAD). PAD is associated with an increased risk of all-cause mortality, cardiovascular (CV) mortality, and major adverse cardiovascular events (MACE) in patients with AF. Furthermore, peripheral atherosclerosis was reported to increase the inflammatory state, which can negatively impact the AF. (Zhu et al., Peripheral artery disease and clinical outcomes in patients with atrial fibrillation: A systematic review and meta-analysis, Clin Cardiol. 2021 August; 44(8): 1050-1057). Optimal therapy remains uncertain in these cases where both antiplatelet and anticoagulant indications exist (Violi, Peripheral artery disease and atrial fibrillation: a potentially dangerous combination. Intern Emerg Med. 2012; 7:213-218; Bencivenga et al. The Management of Combined Antithrombotic Therapy in Patients With Atrial Fibrillation Undergoing Percutaneous Coronary Intervention: A Particularly Complex Challenge, Especially in the Elderly. Front Physiol. 2018; 9:876. doi: 10.3389/fphys.2018.00876). Up to 7% of patients undergoing percutaneous coronary intervention (PCI) will have existing AF. Atrial fibrillation and myocardial infarction (MI) frequently occur together (Rubenstein et al., Atrial Fibrillation in Acute Coronary Syndrome, J Atr Fibrillation. 2012; 5(1):551; Gibson et al., Prevention of Bleeding in Patients with Atrial Fibrillation Undergoing PCI, N Engl J Med. 2016; 375(25):2423-2434; Capodanno et al., Management of antithrombotic therapy in atrial fibrillation patients undergoing PCI, J Am Coll Cardiol 2019; 74:83-99; Capodanno et al., Safety and efficacy of double antithrombotic therapy with non-vitamin K antagonist oral anticoagulants in patients with AF undergoing PCI: a systematic review and meta-analysis, J Am Heart Assoc 2020; 18:9:e017212).

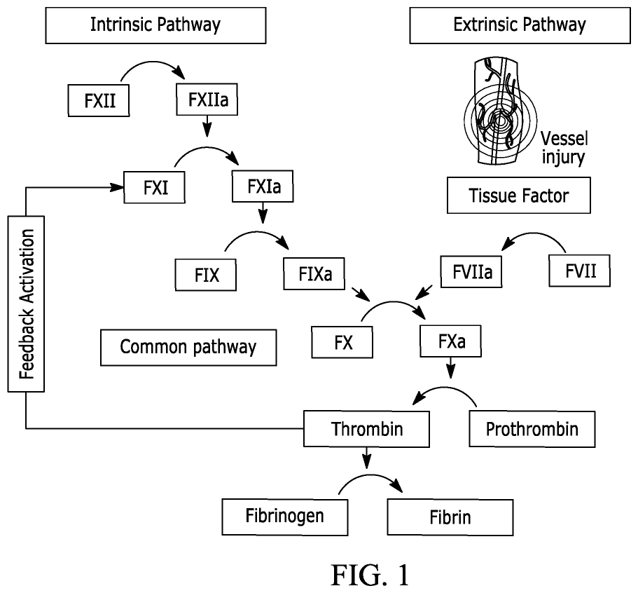

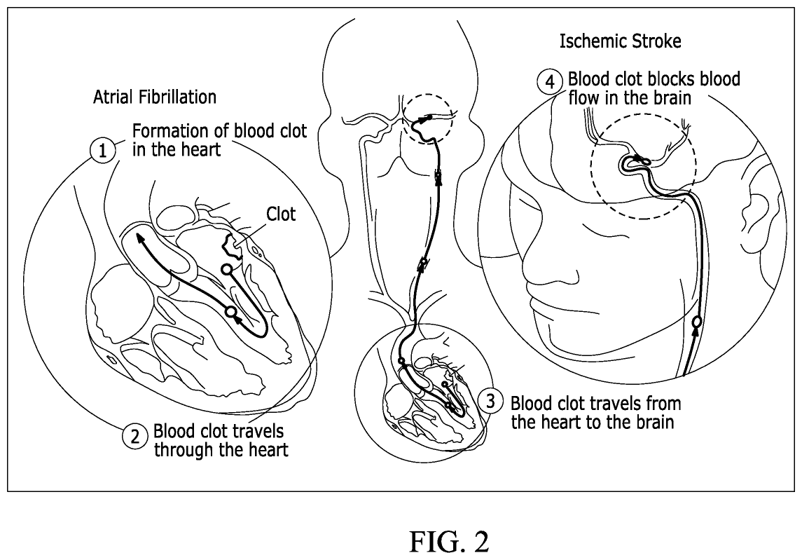

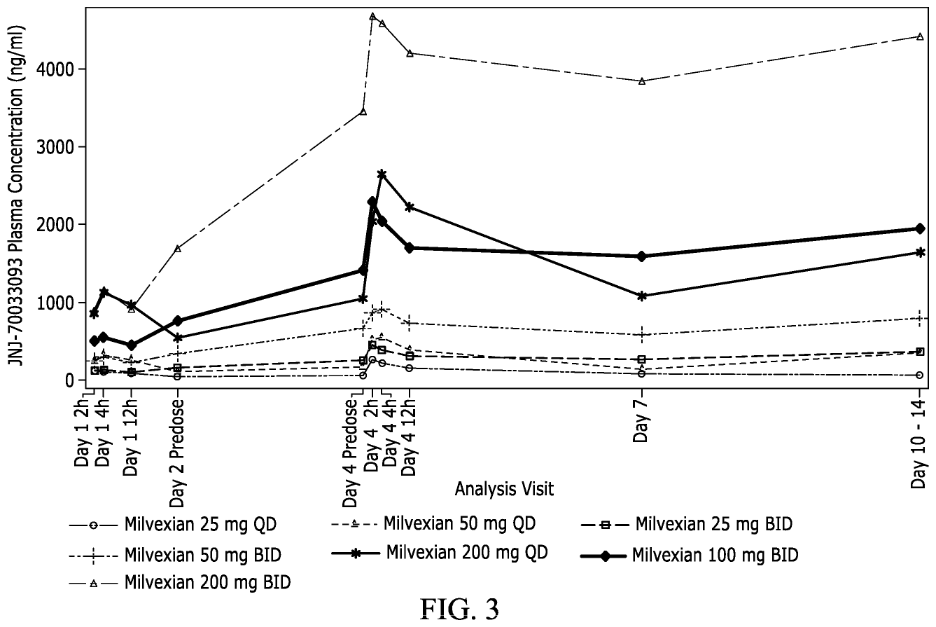

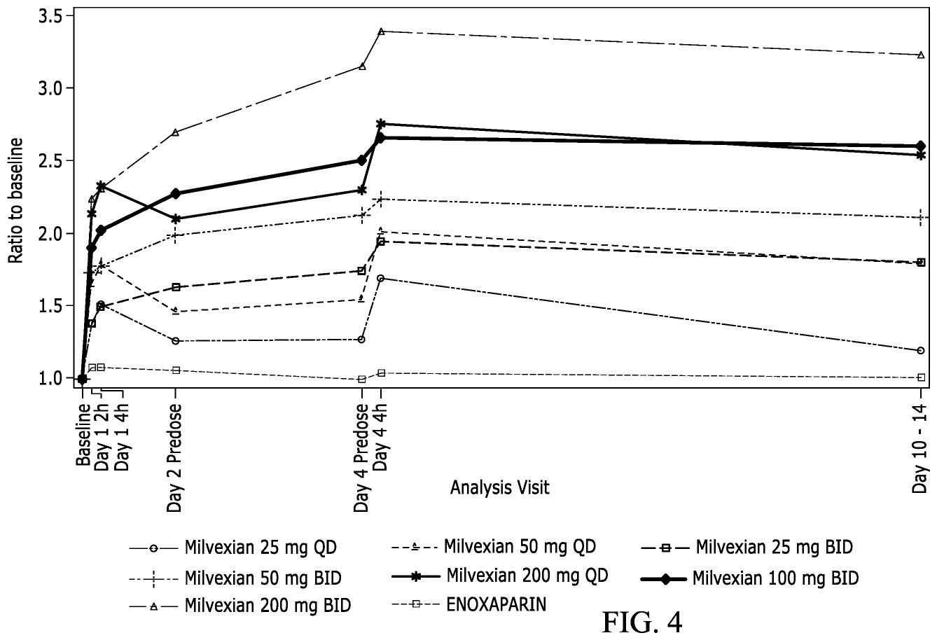

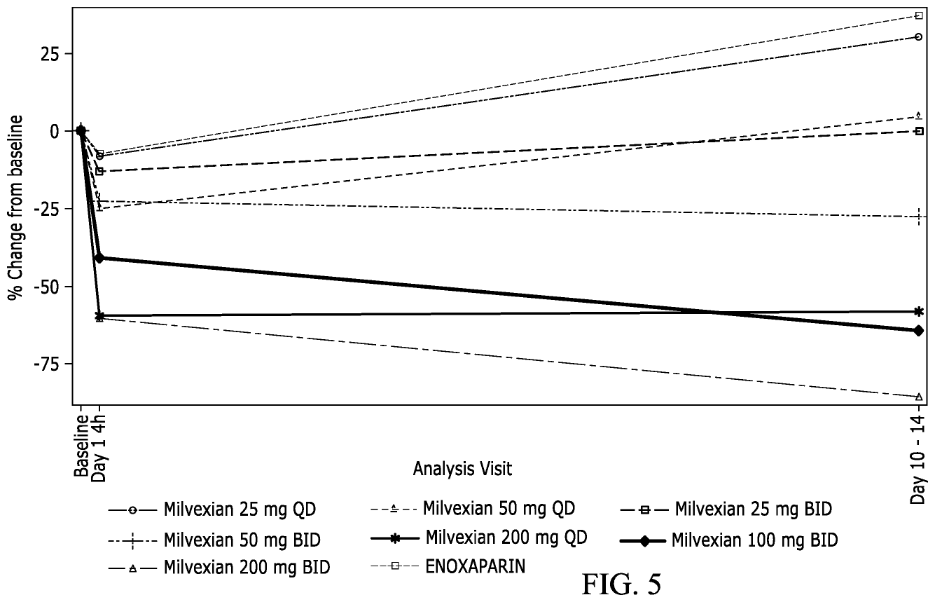

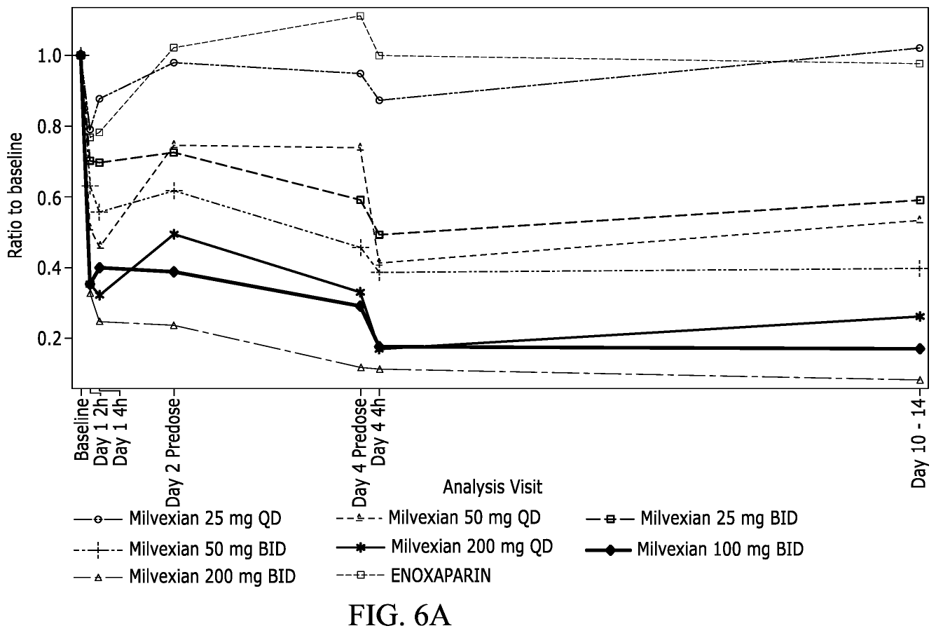

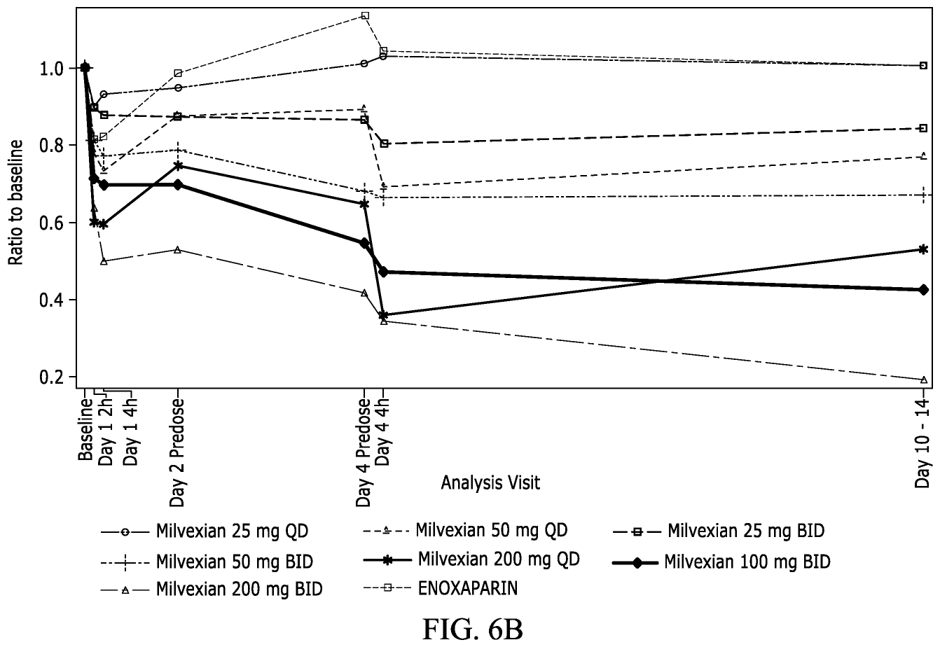

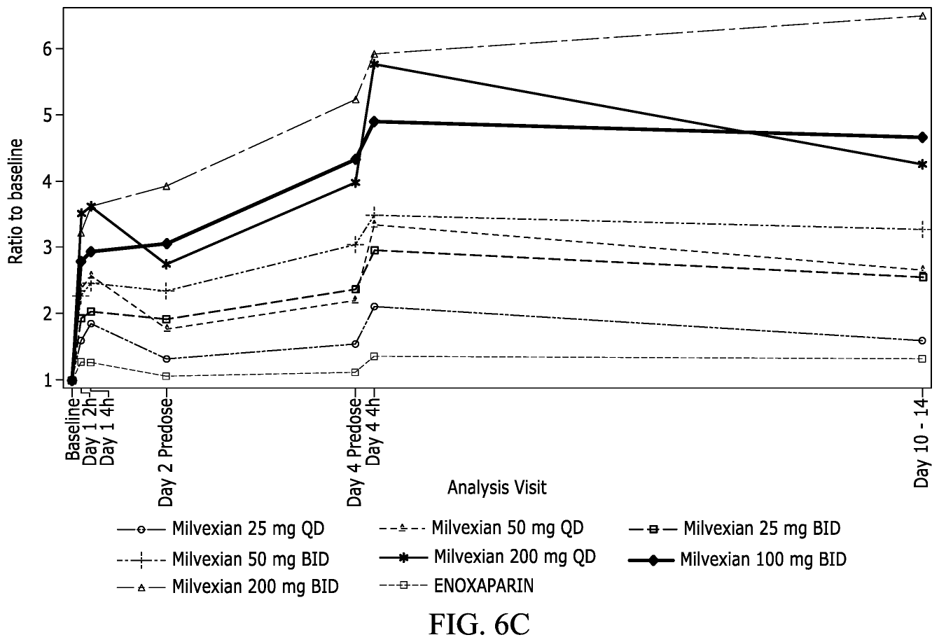

FXIa inhibition has the potential to reduce thrombin generation, prevent thrombus formation and embolism, thus reduce stroke risk in patients with atrial fibrillation (AF), without impairing hemostasis (See FIG. 1). While Factor Xa inhibitors block the tissue factor-activated coagulation process required for hemostasis as well as the intrinsic pathway, Factor XIa inhibition blocks thrombus formation through the intrinsic pathway (or the ‘contact’ system) which may be dispensable for normal hemostasis, and through an amplification pathway of thrombin generation which appears to contribute to pathological thrombus formation (See FIG. 1). Therefore, Factor XIa inhibitors have the potential for an improved benefit to risk profile than inhibitors of Factor Xa or thrombin. (Weitz, et al., Effect of Osocimab in Preventing Venous Thromboembolism Among Patients Undergoing Knee Arthroplasty: The FOXTROT Randomized Clinical Trial, JAMA. 2020; 323(2):130-139). In addition, atrial fibrillation is a chronic disease and therefore the development of oral formulations is key for patient compliance and patient adherence (Bentley et al., Drugs in Phase I and Phase II Clinical Development for the Prevention of Stroke in Patients with Atrial Fibrillation, Expert Opinion On Investigational Drugs, 2021, Vol. 30, 1057-1069, https://doi.org/10.1080/13543784.2021.1897786).

4,114 participants have been included and exposed to the study intervention (milvexian, placebo, or comparator) in the milvexian clinical program. Of the 4,114 participants, 3,229 received milvexian, of which 660 participants were exposed to milvexian in the Phase 1 studies and 2,569 participants were exposed to milvexian in the Phase 2 and 2a studies. Four types of serious bleeding in the milvexian clinical development program were assessed as adverse drug reactions: gastrointestinal bleeding, procedural hemorrhage, nervous system disorder bleeding (hemorrhagic transformation of ischemic stroke and subdural hematoma) and hematuria. The result of Phase II clinical trial of milvexian in patients undergoing TKR (total knee replacement) was published in 2021 (Weitz, J. I.; et al. Milvexian for the Prevention of Venous Thromboembolism. N. Engl. J. Med. 2021, 385, 2161-2172). The results of Phase II clinical trial using milvexian in addition to a single or dual antiplatelet therapy (SAPT/DAPT) for preventing non-cardioembolic, non-lacunar ischemic stroke was published in 2023 (Sharma et al., Safety and efficacy of factor XIa inhibition with milvexian for secondary stroke prevention (AXIOMATIC-SSP): a phase 2, international, randomised, double-blind, placebo-controlled, dose-finding trial, The Lancet Neurology, 2023, vol. 23, pp-46-59). Milvexian in the two Phase II clinical trials (AXIOMATIC-TKR and AXIOMATIC-SSP) focused on different patient populations.

As used herein, the term “atrial fibrillation” (AF) refers to a supraventricular tachyarrhythmia characterized by uncoordinated atrial activation with consequent deterioration of atrial mechanical function. As used herein, atrial fibrillation also includes atrial flutter. AF features atrial wavelets propagating in different directions, causing disorganized atrial depolarization without effective atrial contraction. On the electrocardiogram, AF is described by the replacement of consistent P waves by rapid oscillations (brillatory (‘f’) waves) that vary in size, shape, and timing, at a rate of 350-600 beats/min, associated with an irregular, frequently rapid ventricular response when atrioventricular conduction is intact. The ventricular response to AF depends on the electrophysiological properties of the atrioventricular node, the level of vagal and sympathetic tone, and the action of drugs. By convention, an episode lasting at least 30 seconds or for an entire 12-lead electrocardiogram is considered diagnostic for clinical AF. The most recent classification proposed by the European Society of Cardiology (ESC) 3 is the following: (1) First diagnosed AF: when a patient presents AF for the first time, irrespective of the duration of the arrhythmia or the presence and severity of AF-related symptoms. (2) Paroxysmal AF: when AF is self-terminating, usually within 48 hours. Although AF paroxysms may continue for up to 7 days, the 48-hour time point is clinically important because after this the likelihood of spontaneous conversion is low and anticoagulation must be considered. (3) Persistent AF: when AF episodes either last longer than 7 days or require termination by either pharmacological or electrical cardioversion. (4) Long-standing persistent AF: when AF has lasted for at least 1 year when adopting a rhythm control strategy is decided. (5) Permanent AF: when the presence of AF is accepted by the patient (and the physician). Atrial flutter occurs when certain electrical signals do not reach the ventricles of the heart. The rapid heartbeat associated with atrial flutter also increases the risk of developing blood clots and stroke. Often, AFib and atrial flutter occur at the same time.

As used herein, the terms “patients with atrial fibrillation”, “patients with atrial fibrillation or flutter”, “patients having a history of atrial fibrillation or atrial flutter”, “patients with a history of or a current atrial fibrillation or atrial flutter” or “patients with a recent history of or a current atrial fibrillation or atrial flutter” or “patients with paroxysmal or persistent atrial fibrillation or atrial flutter” or “patients with a history of, or a current paroxysmal or persistent atrial fibrillation or atrial flutter” or “patients with a recent history of, or a current paroxysmal or persistent atrial fibrillation or atrial flutter” or “patients with paroxysmal or intermittent atrial fibrillation or atrial flutter and a recent episode of atrial fibrillation or atrial flutter, who are in sinus rhythm or who will be cardioverted” or “patients with paroxysmal or persistent atrial fibrillation or atrial flutter and a recent episode of atrial fibrillation or atrial flutter, who are in sinus rhythm or who will be cardioverted” means a patient who, in the past, has presented one or more episodes of atrial fibrillation or atrial flutter and/or who is suffering from atrial fibrillation or atrial flutter at the time the milvexian or a pharmaceutically acceptable salt thereof is used and is eligible for anticoagulation. More particularly, this term means patients with documentation of having been in both atrial fibrillation or flutter and sinus rhythm within the last 6 months preceding the start of treatment. Patients could be either in sinus rhythm, or in atrial fibrillation or atrial flutter at the time the milvexian or a pharmaceutically acceptable salt thereof is initiated. In some embodiments, the patient has a recent history of, or a current, non-permanent atrial fibrillation or atrial flutter. In some embodiments, the patient has paroxysmal atrial fibrillation (i.e., AF that occurs intermittently and stops on its own within seven days). In some embodiments, the patient has persistent atrial fibrillation (i.e., AF that lasts longer than seven days, and may require electric shocks to the heart to restore normal rhythm). In some embodiments, the patient has long-standing persistent atrial fibrillation (i.e., AF that is persistent, but lasts longer than 1 year). In some embodiments, the patient has permanent/chronic atrial fibrillation (i.e., the patient is always in AF, and all attempts for restoring sinus rhythm have failed). In some embodiments, the patients, who have a recent history of atrial fibrillation or atrial flutter, also have concomitant coronary artery disease (CAD) and/or peripheral artery disease (PAD). In some embodiments, the patients, who have a recent history of atrial fibrillation or atrial flutter that is paroxysmal, or sustained, or not reversible, were diagnosed within one year prior to the first dose of milvexian treatment regimen as described herein. Among the patients, notably patients having a history of atrial fibrillation or atrial flutter, the patients further have one or more of Category (A) risk factor selected from (i) age greater than or equal to 75 years, or (ii) history of a clinical symptomatic stroke (e.g., History of symptomatic or silent stroke of any type (ischemic, hemorrhagic, lacunar, or undetermined, or cerebral microbleeds (CMB). If ischemic stroke, it must be >7 days prior to the first dose of milvexian treatment regimen as described herein. Hemorrhagic strokes/transformations must occurred equal to or longer than 3 months); and/or two or more of concomitant or comorbid Category (B) cardiovascular risk factor selected from the group consisting of: (i) age between 65 and 74 years; (ii) hypertension (e.g., use of antihypertensive medications within 6 months before screening, or persistent SBP >140 mmHg or DBP >90 mmHg); (iii) diabetes mellitus (e.g., history of diabetes mellitus and current use of antidiabetic medication(s)); (iv) atherosclerotic vascular diseases (hardening of the arteries, CAD, MI, PAD, PCI, CABG); and (v) congestive heart failure (e.g., symptomatic heart failure including history of hospitalization with heart failure as primary cause regardless of ejection fraction (EF), or EF <40% regardless of history of heart failure hospitalization (most recent and assessed less than 1 year prior to the first dose of milvexian treatment regimen as described herein)). A normal ejection fraction is 50% or higher. An ejection fraction below 40% means the heart isn't pumping enough blood and may be failing. The terms “persistent” and “intermittent” are used interchangeably. More particularly, this term means patients who are medically stable and appropriate for chronic antithrombotic treatment. The term CMBs are defined as rounded foci of <10 mm in size that appear hypointense and distinct from vascular flow voids, leptomeningeal hemosiderosis, or non-hemorrhagic subcortical mineralization on T2*-weighted MRI.

Patients in “permanent atrial fibrillation or flutter” are patients that have all scheduled ECGs in this rhythm throughout the period the dronedarone or a pharmaceutically acceptable salt thereof is administered.

As used herein, the term “activated partial thromboplastin time (aPTT)” refers to a measure of the intrinsic and final common pathways of the coagulation cascade. It represents the time, in seconds, for plasma to clot after addition of phospholipid, an intrinsic pathway activator, and calcium. The name ‘Activated Partial Thromboplastin Time’ comes from the original form of the test in which only the phospholipid concentration of the test was controlled (as opposed to the phospholipid and the surface activator concentrations) and the name ‘partial thromboplastin’ was applied at the time to phospholipid preparations that accelerated clotting but did not correct the prolonged clotting times of hemophilic plasma. The term ‘partial’ means phospholipid is present but no tissue factor. The normal and reference ranges vary depending on reagent and instrument combinations, particularly with the phospholipid composition.

In some embodiments, aPTT is measured as follows: Plasma samples are incubated with Actin FS aPTT assay reagent containing a standard amount of phospholipid and contact activator (ellagic acid) which activates the intrinsic coagulation pathway. After incubating for 3 minutes, calcium chloride is added to initiate coagulation and formation of a fibrin clot is measured optically. The time to clot formation (measured in seconds) is reported as the Activated Partial Thromboplastin Time (aPTT).

Administration of multiple oral doses of milvexian to healthy human subjects resulted in a dose- and concentration-dependent prolongation of aPTT. The maximal mean change of aPTT from baseline was approximately a 1.1- to 4.1-fold increase after QD doses of 5 to 500 mg for 2 weeks and a 3.4-fold increase after BID dose of 200 mg for 2 weeks. Prothrombin time was not impacted by the administration of a single or multiple doses of milvexian to human subjects, with a maximal mean percent change from baseline of approximately 5%. As used herein, a prothrombin time (PT) test measures how long it takes for a clot to form in a blood sample).

As used herein, “Factor XI Clotting Activity” is determined utilizing an aPTT-based 1-stage clotting time assay. Serial dilutions of normal pooled plasma are mixed with FXI-depleted plasma and the clotting times are measured according to standard aPTT protocol, to establish a reference range. Subject test plasma is treated in the same way and compared with the reference plasma.

In some embodiments, the Factor XI Clotting Activity is measured as follows: Factor XI (FXI) activity is measured using a modification of the activated partial thromboplastin time (aPTT) using Actin FS (Siemens Healthcare) on the Siemens BCS®XP analyzer. A 6-point calibration curve (~5-150%) is prepared using a secondary calibrator (Standard Human Plasma, Siemens Healthcare Diagnostics Inc.) with a known concentration of human FXI assigned by the manufacturer. The reference standard, at approximately 100%, is diluted by the BCS®XP analyzer in saline to generate pre-selected calibration levels of FXI. The calibration curve is plotted with FXI activity in percent (%) on the x-axis and clotting time in seconds on the y-axis. A log/lin regression curve fit is used. The samples to be tested are mixed with FXI deficient plasma (containing less than 1% FXI and at least 75% of all the other factors) to normalize all other factors. APTT reagent (Actin FS) is added and the mixture is incubated. Following incubation, calcium chloride is added to the mixture and the time to clot formation (measured optically) is compared to the time on the calibration curve. Samples are tested at the base dilution (1:10) prepared by the BCS®XP in saline.

In some embodiments, “plasma half-life” is used interchangeably with terminal half-life (t½).

As used herein, “Thrombin Generation Assay (TGA)” is a global coagulation assay that evaluates the thrombogenic capacity of a plasma sample and has been proposed that it may better reflect prothrombotic or hemorrhagic states than conventional clotting assays. In the traditional TGA, coagulation of citrated plasma is initiated through the extrinsic pathway by adding tissue factor, phospholipids, and calcium. For this study, in the in-vitro thrombin generation assay (TGA) using human platelet-rich plasma, and a kaolin slurry was used instead to initiate coagulation through the contact activation pathway. Thrombin generation is continuously monitored through the product released by cleavage of a thrombin-specific fluorogenic substrate. The generated thrombogram is used to determine several relevant parameters, including endogenous thrombin potential, defined as the area under the thrombin concentration vs. time curve. In some embodiments, administration of a single dose or multiple oral doses of milvexian to a human subject results in inhibition of thrombin generation via the intrinsic pathway, but only minimal inhibition of thrombin generation when initiated by the extrinsic pathway.

As used herein, “preventing” refers to reducing the risk of occurrence. In some embodiments, preventing encompasses eliminating the risk of occurrence (i.e., reducing the risk of occurrence to zero). As such, “prevention” covers the preventive treatment aimed at reducing the probability of the occurrence of a clinical disease-state. In some embodiments, for any of the method described herein, “preventing” encompasses the reduction of recurrence of a clinical disease-state.

In some embodiments, “preventing” is synonymous with “reducing the risk” or “reducing the incidence rate” of an adverse cardioembolic event occurring. Reducing the risk or reducing the incidence rate means that there is numerical and/or a statistically-significant reduction or lowering in occurrence of the adverse cerebrovascular event by at least 1% or greater. Preferably, this reduction is by 2% or greater, 3% or greater, 4% or greater, 5% or greater, 6% or greater, 7% or greater, 10% or greater, 20% or greater, 26% or greater, 34% or greater, 50% or greater, 64% or greater and 74% or greater. These reductions include confidence intervals equals or greater than 50%, equals or greater than 75%, equals or greater than 80%, equals or greater than 90%, equals or greater than 95%, equals or greater than 98% and equals or greater than 99%. Confidence intervals equal to greater than 95% are preferred.

Within the scope of this disclosure, “prophylaxis” is the protective treatment of a disease state to reduce and/or minimize the risk and/or reduction in the risk of recurrence of a disease state by administering to a patient a therapeutically effective amount of milvexian or a pharmaceutically acceptable salt, or a solvate thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, the term “treating” refers to ameliorating the signs or symptoms of a disease or condition in a patient and/or preventing a disease or condition in a patient. As used herein, unless otherwise noted, the terms “treating”, “treatment” and the like, shall include the management and care of a patient for the purpose of combating a disease, condition, or disorder and includes the administration of milvexian to prevent the onset of the symptoms or complications, alleviate the symptoms or complications, or eliminate the disease, condition, or disorder. As such, “treating” or “treatment” cover the treatment of a disease-state in a human, and include: (a) inhibiting the disease-state, i.e., arresting its development; and/or (b) relieving the disease-state, i.e., causing regression of the disease state.

As used herein, a “risk factor” is a demographic factor that influences the underlying risk of an event independently of any drug treatment.

As used herein, the term “cerebrovascular disorders” or “cerebrovascular disease” refers to the neurologic problems resulting from the disruption of the blood flow to the brain that leads to damage or death of brain cells from the lack of oxygen. In some embodiments, the cerebrovascular disorders include transient ischemic attack. In some embodiments, the cerebrovascular disorders include stroke. How a stroke or transient ischemic attack affects the body depends on precisely where in the brain the blood supply was cut off.

In some embodiments, the term “stroke” refers to an acute episode of neurologic dysfunction that caused the death of brain tissue (cerebral infarction) resulting from lack of blood flow and insufficient oxygen to the brain. A stroke can either be ischemic or hemorrhagic. In an ischemic stroke, the blood supply to part of the brain is cut off because a blood clot has blocked a blood vessel due to either a thrombus formed locally in an abnormal artery (e.g., atherosclerosis) or an embolus that formed upstream and traveled through the bloodstream. In a hemorrhagic stroke, a blood vessel bursts, preventing normal blood flow and allowing blood to leak into an area of the brain and destroy it. Most strokes begin suddenly, develop rapidly and cause brain damage within minutes (complete stroke). Less commonly, strokes may continue to worsen for several hours to days as a steadily enlarging area of brain tissue dies (stroke in evolution). Ischemic stroke may be lacunar or non-lacunar in nature. Ischemic stroke may be cardioembolic in origin if the ischemic stroke is attributable to arterial occlusion from embolus that presumably arose from the blood clots formed in the heart or on one of its valves. In some embodiments, the term “ischemic stroke” and “symptomatic ischemic stroke” may be used interchangeably.

The severity of stroke is measured using the National Institutes of Health Stroke Scale (NIHSS) scores in clinical trials (Kamel et al., Validation of the International Classification of Diseases, Tenth Revision Code for the National Institutes of Health Stroke Scale Score. Circ Cardiovasc Qual Outcomes, 2023, vol. 16, e009215). Stroke severity is categorized as follows: 1-4 for minor stroke, 5-15 for moderate stroke, 16-20 for moderate to severe stroke, and 21-42 for severe stroke.

In some embodiments, ischemic stroke refers to a neurological deficit attributable to a non-lacunar, acute brain infarction detected by neuroimaging (CT or MRI) and relevant to the clinical symptoms.

In some embodiments, the ischemic stroke is further characterized by a National Institutes of Health Stroke Score (NIHSS)≤7.

In other embodiments, the ischemic stroke is further characterized by a National Institutes of Health Stroke Score (NIHSS) of 8-15.

In other embodiments, the ischemic stroke is further characterized by a National Institutes of Health Stroke Score (NIHSS)≤15.

In still other embodiments, an ischemic stroke is further characterized by a Modified Rankin Score. For example, in some embodiments an ischemic stroke is further characterized by a Modified Rankin Score (mRS) of ≤3, of ≤4, of ≤5, or of ≤6.

As used herein, the term “non-CNS systemic embolism” refers to an abrupt vascular insufficiency associated with clinical or radiologic evidence of arterial occlusion in the absence of other likely mechanisms. The term “systemic embolism” is used interchangeably with “non-CNS systemic embolism.”

As used herein, “pharmaceutically acceptable salt” refers to derivatives wherein a compound is modified by making an acid or a basic salt thereof. Examples of pharmaceutically acceptable salts include, but are not limited to, mineral or organic acid salts of basic groups such as amines; and alkali or organic salts of acidic groups such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic. The pharmaceutically acceptable salts of milvexian can be synthesized using conventional chemical methods. Generally, such salts can be prepared by reacting milvexian with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, Pa. (1990), the disclosure of which is hereby incorporated by reference.”

As used herein, the term “standard of care” refers to a treatment process that is generally accepted by medical experts as a proper treatment for a certain type of disease (e.g., stroke) and that is widely used by healthcare professionals.

As used herein, the term “hemostasis” means normal blood clotting activity.

The definition of major bleeding described below is adapted from the International Society on Thrombosis and Hemostasis (ISTH) definition. Major bleeding event is defined as a bleeding event that is: Acute clinically overt bleeding accompanied by one or more of the following: A decrease in hemoglobin (Hgb) of 2 g/dL or more over a 24-hour period; A transfusion of 2 or more units of packed red blood cells; Bleeding that occurs in at least one of the following critical sites: Intracranial, Intra-spinal, Intraocular (within the corpus of the eye; thus, a conjunctival bleed is not an intraocular bleed), Pericardial, Intra-articular, Intramuscular with compartment syndrome, or Retroperitoneal; or Bleeding that is fatal.

In some embodiments, a clinically relevant non-major (CRNM) bleeding event is defined as an acute or sub-acute clinically overt bleeding that does not satisfy the criteria for major bleeding and that leads to either 1) hospital admission for bleeding or 2) physician guided medical or surgical treatment for bleeding or 3) a change in antithrombotic therapy.

In some embodiments, the “Bleeding Academic Research Consortium (BARC) criteria are as set forth in Roxana Mehran, et al. Standardized Bleeding Definitions for Cardiovascular Clinical Trials, Circulation. 2011; 123:2736-2747, which is incorporated by reference herein.

In other embodiments, the “Bleeding Academic Research Consortium (BARC) criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the GUSTO (Global Use of Streptokinase and t-PA for Occluded Coronary Arteries) criteria are as set forth in “An international randomized trial comparing four thrombolytic strategies for acute myocardial infarction.” The GUSTO investigators. N Engl J Med 1993; 329:673-82, which is incorporated by reference herein.

In some embodiments, the GUSTO criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the TIMI criteria are as set forth in Rao A K, Pratt C, Berke A, et al. Thrombolysis in Myocardial Infarction (TIMI) Trial-phase I: hemorrhagic manifestations and changes in plasma fibrinogen and the fibrinolytic system in patients treated with recombinant tissue plasminogen activator and streptokinase. J Am Coll Cardiol 1988; 11:1-11. which is incorporated by reference herein.

In some embodiments, the TIMI criteria are as set forth in Pascal Vranckx, et al. Validation of BARC Bleeding Criteria in Patients With Acute Coronary Syndromes J Am Coll Cardiol 2016; 67:2135-44, which is incorporated by reference herein.

In some embodiments, the “Bleeding Academic Research Consortium (BARC) Type 3 criteria” are: a. Overt bleeding plus hemoglobin drop of 3 to <5 g/dL (provided hemoglobin drop is related to bleed); transfusion with overt bleeding; b. Overt bleeding plus hemoglobin drop ≥5 g/dL (provided hemoglobin drop is related to bleed); cardiac tamponade; bleeding requiring surgical intervention for control; bleeding requiring IV vasoactive agents; or c. Intracranial hemorrhage confirmed by autopsy, imaging, or lumbar puncture; intraocular bleed compromising vision. See, e.g., Roxana Mehran, et al. Standardized Bleeding Definitions for Cardiovascular Clinical Trials, Circulation. 2011; 123:2736-2747.

In some embodiments, the “Bleeding Academic Research Consortium (BARC) Type 5 criteria” are: a. Probable fatal bleeding; or b. Definite fatal bleeding (overt or autopsy or imaging confirmation). See, e.g., Roxana Mehran, et al. Standardized Bleeding Definitions for Cardiovascular Clinical Trials, Circulation. 2011; 123:2736-2747.

In some embodiments, the “Bleeding Academic Research Consortium (BARC) Type 2 criteria” are: any clinically overt sign of hemorrhage that “is actionable” and requires diagnostic studies, hospitalization, or treatment by a health care professional. See, e.g., Roxana Mehran, et al. Standardized Bleeding Definitions for Cardiovascular Clinical Trials, Circulation. 2011; 123:2736-2747.

In some embodiments, the “ISTH criteria (major bleeding or clinically-relevant non-major bleeding (CRNM)) criteria” are as follows:
ISTH major bleeding in non-surgical patients is defined as having a symptomatic presentation and:
   i. Fatal bleeding, and/or
   ii. Bleeding in a critical area or organ, such as intracranial, intraspinal, intraocular, retroperitoneal, intra-articular or pericardial, or intramuscular with compartment syndrome, and/or
   iii. Bleeding causing a fall in hemoglobin level of 20 g L⁻¹ (1.24 mmol L⁻¹) or more, or leading to transfusion of two or more units of whole blood or red cell
ISTH CRNM bleeding is any sign or symptom of hemorrhage (e.g., more bleeding than would be expected for a clinical circumstance, including bleeding found by imaging alone) that does not fit the criteria for the ISTH definition of major bleeding but does meet at least one of the following criteria:
   i. requiring medical intervention by a healthcare professional
   ii. leading to hospitalization or increased level of care
   iii. prompting a face to face (i.e., not just a telephone or electronic communication) evaluation

In some embodiments, CRNM bleeding refers to acute or sub-acute overt bleeding that leads to either:
hospital admission for bleeding, or
physician guided medical or surgical treatment for bleeding, or
a change in antithrombotic therapy.

It will be understood that the use of terms of approximation, such as “approximately” or “about,” when referring to, for example, amounts, durations of time, extents of effects, and the like, may be a number that is within 0% (greater or less than) (i.e., may be a number that is the specified number), may be a number that is within 5% (greater or less than), or within 7.5% (greater or less than), or within 10% (greater or less than), or within 12.5% (greater or less than), or within 15% (greater or less than), or within 17.5% (greater or less than), or within 20% (greater or less than) of the specified number.

### Methods of the Disclosure

In some aspects, the disclosure provides methods for preventing one or more of stroke and non-central nervous system (CNS) systemic embolism events in a human patient with a history of atrial fibrillation or flutter, wherein the method comprises administering to the human patient a composition comprising about 50 mg, about 75.0 mg, about 87.5 mg, or about 100 mg of milvexian (or a pharmaceutically acceptable salt or solvate thereof, on a milvexian basis); wherein the composition is administered twice daily.

In some aspects, the disclosure provides methods for preventing one or more of stroke and non-central nervous system (CNS) systemic embolism events in a human patient with a history of atrial fibrillation or flutter, wherein the method comprises administering to the human patient a composition comprising about 50 mg, or about 100 mg of milvexian (or a pharmaceutically acceptable salt or solvate thereof, on a milvexian basis); wherein the composition is administered twice daily.

In some aspects, the disclosure provides methods for preventing one or more of stroke and non-central nervous system (CNS) systemic embolism events in a human patient with a history of atrial fibrillation or flutter, wherein the method comprises administering to the human patient a composition comprising about 50 mg of milvexian (or a pharmaceutically acceptable salt or solvate thereof, on a milvexian basis); wherein the composition is administered twice daily.

In some aspects, the disclosure provides methods for preventing one or more of stroke and non-central nervous system (CNS) systemic embolism events in a human patient with a history of atrial fibrillation or flutter, wherein the method comprises administering to the human patient a composition comprising about 75.0 mg of milvexian (or a pharmaceutically acceptable salt or solvate thereof, on a milvexian basis); wherein the composition is administered twice daily.

In some aspects, the disclosure provides methods for preventing one or more of stroke and non-central nervous system (CNS) systemic embolism events in a human patient with a history of atrial fibrillation or flutter, wherein the method comprises administering to the human patient a composition comprising about 87.5 mg of milvexian (or a pharmaceutically acceptable salt or solvate thereof, on a milvexian basis); wherein the composition is administered twice daily.

In some aspects, the disclosure provides methods for preventing one or more of stroke and non-central nervous system (CNS) systemic embolism in a human patient with atrial fibrillation or flutter, wherein the method comprises administering to the human patient a composition comprising about 100 mg of milvexian (or a pharmaceutically acceptable salt or solvate thereof, on a milvexian basis); wherein the composition is administered twice daily (BID).

In some aspects, the disclosure provides methods for preventing one or more of stroke and non-central nervous system (CNS) systemic embolism in a human patient with atrial fibrillation or flutter, wherein the method comprises administering to the human patient about 100 mg of milvexian twice daily.

In some aspects, for any of the method described herein, treatment regimen may be administered once daily (QD).

In some aspects, for any of the method described herein, the treatment regimen may further comprise a standard-of-care antiplatelet therapy. In some embodiments, the antiplatelet therapy is selected from adenosine diphosphate (ADP) receptor inhibitors (e.g., clopidogrel, ticagrelor, ticlopidine, prasugrel) for making platelets less sticky; adenosine reuptake inhibitors (e.g., dipyridamole) for blocking enzymes involved in clotting; glycoprotein platelet inhibitors (e.g., abciximab, eptifibatide, tirofiban) for blocking substances that help clots stick together; phosphodiesterase inhibitors (e.g., cilostazol) for widening blood vessels and stopping platelets from sticking together; or protease-activated receptor (PAR-1) antagonist (e.g., vorapaxar) for blocking a substance on platelets that helps them clot.

In some embodiments, the standard-of-care comprises single antiplatelet therapy (SAPT).

In some embodiments, the single antiplatelet therapy comprises aspirin, or a P2Y12 inhibitor selected from clopidogrel, ticagrelor, or prasugrel.

In some embodiments, the standard-of-care comprises dual antiplatelet therapy (DAPT).

In some embodiments, the standard-of-care comprises dual antiplatelet therapy (DAPT) for 21 days, followed by single antiplatelet therapy (SAPT) thereafter.

In some embodiments, the single antiplatelet therapy is aspirin.

In some embodiments of the single antiplatelet therapy, aspirin is administered in an amount of 50-150 mg daily, such as, for example, one of: about 50 mg, about 55 mg, about 60 mg, about 65 mg, about 70 mg, about 75 mg, about 80 mg, about 81 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg, daily.

In some embodiments of the single antiplatelet therapy, the aspirin is administered in an amount of about 75 mg to about 100 mg daily, such as, for example, one of: about 75 mg, about 80 mg, about 81 mg, about 85 mg, about 90 mg, about 95 mg, or about 100 mg, daily.

In some embodiments of the single antiplatelet therapy, the aspirin is administered in an amount of about 75 mg daily.

In some embodiments of the single antiplatelet therapy, the aspirin is administered in an amount of about 81 mg daily.

In some embodiments of the single antiplatelet therapy, the aspirin is administered in an amount of about 100 mg daily.

In other embodiments, the single antiplatelet therapy is a P2Y12 inhibitor.

In some embodiments of the single antiplatelet therapy, the P2Y12 inhibitor is clopidogrel, ticagrelor, or prasugrel.

In some embodiments of the single antiplatelet therapy, the P2Y12 inhibitor is clopidogrel.

In some embodiments of the single antiplatelet therapy, the clopidogrel is administered in an amount of about 75 mg to about 600 mg daily, such as, for example, one of: about 75 mg, about 150 mg, about 225 mg, about 300 mg, about 375 mg, about 450 mg, about 525 mg, or about 600 mg daily.

In some embodiments of the single antiplatelet therapy, the P2Y12 inhibitor is ticagrelor.

In some embodiments, the single antiplatelet therapy is ticlopidine.

In some embodiments, the ticlopidine is administered in amounts an amount of about 250 mg to about 500 mg daily, such as, for example, about 150 mg, or about 500 mg daily.

In other embodiments of the disclosed methods, the antiplatelet therapy comprises dual antiplatelet therapy.

In some embodiments, the dual antiplatelet therapy is aspirin and ticlopidine.

In some embodiments, the dual antiplatelet therapy is aspirin and a P2Y12 inhibitor.

In some embodiments of the dual antiplatelet therapy, the aspirin is administered in an amount of about 50 mg to about 150 mg daily, such as, for example, one of: about 50 mg, about 55 mg, about 60 mg, about 65 mg, about 70 mg, about 75 mg, about 80 mg, about 81 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg, daily.

In some embodiments of the dual antiplatelet therapy, the aspirin is administered in an amount of about 75 mg to 100 mg daily, such as, for example, one of about 75 mg, about 80 mg, about 81 mg, about 85 mg, about 90 mg, about 95 mg, or about 100 mg, daily.

In some embodiments of the dual antiplatelet therapy, the aspirin is administered in an amount of about 75 mg daily.

In some embodiments of the dual antiplatelet therapy, the aspirin is administered in an amount of about 81 mg daily.

In some embodiments of the dual antiplatelet therapy, the aspirin is administered in an amount of about 100 mg daily.

In some embodiments of the dual antiplatelet therapy, the P2Y12 inhibitor is clopidogrel, ticagrelor, or prasugrel.

In some embodiments of the dual antiplatelet therapy, the P2Y12 inhibitor is clopidogrel.

In some embodiments of the dual antiplatelet therapy, the clopidogrel is administered in an amount of about 75 mg to about 600 mg daily, such as, for example, one of: about 75 mg, about 150 mg, about 225 mg, about 300 mg, about 375 mg, about 450 mg, 525 mg, or about 600 mg daily.

In some embodiments of the dual antiplatelet therapy, the clopidogrel is administered as a loading dose.

In some embodiments, the loading dose is between about 300 mg to 600 mg daily.

In some embodiments of the dual antiplatelet therapy, the clopidogrel is administered in an amount of about 75 mg daily.

In some embodiments of the dual antiplatelet therapy, the clopidogrel is administered in an amount of about 150 mg daily.

In some embodiments of the dual antiplatelet therapy, the clopidogrel is administered in an amount of about 225 mg daily.

In some embodiments of the dual antiplatelet therapy, the clopidogrel is administered in an amount of about 300 mg daily.

In some embodiments of the dual antiplatelet therapy, the clopidogrel is administered in an amount of about 375 mg daily.

In some embodiments of the dual antiplatelet therapy, the clopidogrel is administered in an amount of about 450 mg daily.

In some embodiments of the dual antiplatelet therapy, the clopidogrel is administered in an amount of about 525 mg daily.

In some embodiments of the dual antiplatelet therapy, the clopidogrel is administered in an amount of about 600 mg daily.

In some embodiments, antiplatelet therapy comprises Aggrenox, i.e., a combination of aspirin and dipyridamole (persantine).

In some embodiments, the antiplatelet therapy comprises a combination of single antiplatelet therapy and dual antiplatelet therapy.

In some embodiments, the dual antiplatelet therapy (ie., aspirin and P2Y12 inhibitor) is administered for more than 90 days (with or without de-escalation to SAPT).

In some embodiments, the dual antiplatelet therapy (ie., aspirin and P2Y12 inhibitor) is administered for less than 90 days and a single antiplatelet therapy is administered thereafter.

In some embodiments of the disclosed methods, the dual antiplatelet therapy is administered for up to 21 consecutive days, and single antiplatelet therapy is administered thereafter.

In some embodiments of the disclosed methods, a more potent P2Y12 inhibitor (i.e., ticagrelor or prasugrel) is administered for up to 90 days and a less potent P2Y12 inhibitor (i.e., clopidogrel) is administered thereafter.

In other aspects, the disclosure provides methods for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a composition comprising 50 mg, or 100 mg of milvexian (or a pharmaceutically acceptable salt or solvate thereof, on a milvexian basis); wherein the composition is administered twice daily.

In some embodiments, the human patient is at least 18 years old.

In other embodiments, the human patient is at least 75 years old.

In other embodiments, the human patient is between 64-74 years old, such as, for example, 64 years old, 65 years old, 66 years old, 67 years old, 68 years old, 69 years old, 70 years old, 71 years old, 72 years old, 73 years old, or 74 years old. In some aspects, the patient has atrial fibrillation or flutter.

In some embodiments, the patient has paroxysmal atrial fibrillation.

In other embodiments, the patient has sustained atrial fibrillation not due to a reversible cause.

In some embodiments, the patient has persistent atrial fibrillation (i.e., AF that lasts longer than seven days, and may require electric shocks to the heart to restore normal rhythm).

In some embodiments, the patient has long-standing persistent atrial fibrillation (i.e., AF that is persistent, but lasts longer than 1 year).

In some embodiments, the patient has permanent/chronic atrial fibrillation (i.e., the patient is always in AF, and all attempts for restoring sinus rhythm have failed).

In some embodiments, the patient has paroxysmal, persistent, or long-standing persistent atrial fibrillation.

In some embodiments, the patient's atrial fibrillation is shown by ECG evidence (eg, 12-lead ECG, rhythm strip, Holter, pacemaker interrogation). In some embodiments, the patient has medical evidence of atrial fibrillation within 1 year before and at least 1 day before the ECG evidence.

In some embodiments, the methods of the disclosure are performed on a patient following electrical cardioversion or ablation.

In some embodiments, the patient has atrial fibrillation.

In other embodiments, the patient has atrial flutter.

In other embodiments, the patient has both atrial fibrillation and atrial flutter.

In some embodiments, the patient has medical evidence of atrial fibrillation or atrial flutter.

In some embodiments, the patient has medical evidence of atrial fibrillation or atrial flutter within the past year 1.

In some embodiments, the patient has atrial fibrillation or flutter, and also has concomitant coronary artery disease (CAD) and/or peripheral artery disease (PAD).

In some embodiments, the patient has atrial fibrillation or flutter, and also has concomitant coronary artery disease (CAD).

In some embodiments, the patient has atrial fibrillation or flutter, and also has concomitant peripheral artery disease (PAD).

In some embodiments, the patient has atrial fibrillation or flutter, and also has concomitant coronary artery disease (CAD) and peripheral artery disease (PAD).

In some aspects, for any of the methods as described herein, wherein milvexian reduces the risk of one or more of stroke and non-central nervous system (CNS) systemic embolism. In some embodiments, the term “reduces the risk” or “reduction of the risk” is used interchangeably with “reduces the incidence rate” or “reduction of incidence rate”.

In some embodiments, for any of the methods as described herein, wherein milvexian reduces the risk of stroke.

In some embodiments, for any of the methods of the disclosure as described herein, wherein milvexian reduces the risk of non-central nervous system (CNS) systemic embolism.

In some embodiments, for any of the methods of the disclosure as described herein, wherein milvexian reduces the risk of stroke and non-central nervous system (CNS) systemic embolism.

In some aspects, for any of the methods as described herein, wherein milvexian reduces the risk of adverse cardiovascular events. In some embodiments, each event is selected from the group consisting of cardiovascular death, myocardial infarction, stroke, and non-central nervous system (CNS) systemic embolism.

In some embodiments, for any of the methods as described herein, wherein milvexian reduces the risk of non-central nervous system (CNS) systemic embolism.

In some embodiments, for any of the methods as described herein, wherein milvexian reduces the risk of non-fatal myocardial infarction.

In other embodiments, for any of the methods as described herein, wherein milvexian reduces the risk of preventing non-fatal stroke.

In other embodiments, for any of the methods as described herein, wherein milvexian reduces the risk of cardiovascular death.

In other embodiments, for any of the methods as described herein, wherein milvexian reduces the risk of non-fatal myocardial infarction, non-fatal stroke, and cardiovascular mortality.

In some aspects, for any of the methods as described herein, wherein milvexian reduces the risk of one or more of all-cause death, myocardial infarction, stroke, and non-CNS systemic embolism.

In some embodiments, for any of the methods as described herein, wherein milvexian reduces the risk of all-cause death.

In some embodiments, for any of the methods as described herein, wherein milvexian reduces the risk of myocardial infarction.

In some embodiments, for any of the methods as described herein, wherein milvexian reduces the risk of stroke.

In some embodiments, for any of the methods as described herein, wherein milvexian reduces the risk of non-CNS systemic embolism.

In some aspects, for any of the methods as described herein, wherein milvexian reduces the risk of cardiovascular death, myocardial infarction, stroke, acute limb ischemia (ALI [unanticipated revascularization (including amputation for ischemic limb)]), or urgent hospitalization for vascular cause of ischemic nature (including DVT and PE) in a human patient with atrial fibrillation or atrial flutter.

In some embodiments, for any of the methods as described herein, wherein milvexian reduces the risk of cardiovascular death in a human patient with atrial fibrillation or atrial flutter.

In some embodiments, for any of the methods as described herein, wherein milvexian reduces the risk of myocardial infarction in a human patient with atrial fibrillation or atrial flutter.

In some embodiments, for any of the methods as described herein, wherein milvexian reduces the risk of stroke in a human patient with atrial fibrillation or atrial flutter.

In some embodiments, for any of the methods as described herein, wherein milvexian reduces the risk of a non-CNS embolism in a human patient with atrial fibrillation or atrial flutter.

In some embodiments, for any of the methods as described herein, wherein milvexian reduces the risk of acute limb ischemia (ALI [unanticipated revascularization (including amputation for ischemic limb)]) in a human patient with atrial fibrillation or atrial flutter.

In some embodiments, for any of the methods as described herein, wherein milvexian reduces the risk of urgent hospitalization for vascular cause of ischemic nature (including DVT and PE) in a human patient with atrial fibrillation or atrial flutter.

In some embodiments, for any of the methods as described herein, wherein milvexian reduces the risk of a condition that is stroke or non-CNS systemic embolism in a human patient with atrial fibrillation or atrial flutter.

In some embodiments, for any of the methods as described herein, wherein milvexian reduces the risk of cardiovascular death, myocardial infarction, stroke, or non-CNS embolism in a human patient with atrial fibrillation or atrial flutter.

In some embodiments, for any of the methods as described herein, wherein milvexian reduces the risk of a condition that is all-cause death, myocardial infarction, stroke, or non-CNS embolism in a human patient with atrial fibrillation or atrial flutter.

In some embodiments, for any of the methods as described herein, wherein milvexian reduces the risk of a condition that is cardiovascular death, myocardial infarction, stroke, acute limb ischemia (ALI [any unanticipated revascularization (including amputation for ischemic limb)]), or urgent hospitalization for vascular cause of ischemic nature (including thrombotic events: deep vein thrombosis (DVT) and pulmonary embolism [PE]) in a human patient with atrial fibrillation or atrial flutter.

The primary efficacy endpoint for the clinical trial described in Example 1 below is the time to first occurrence of the composite endpoint of stroke and non-CNS systemic embolism (SE). Primary efficacy endpoint will be analyzed using the Cox Proportional Hazards model, stratified by antiplatelet use, with treatment as the only covariate to provide a point estimate and 95% confidence interval for the hazard ratio (HR). The model assumes proportional hazards and non-informative censoring.

The Cox Proportional Hazards model is as the following:

$$\begin{matrix}{H}_{0} : HR \geq Δ \\ {H}_{1} : HR < Δ\end{matrix}$$

where the HR represents the hazard ratio of milvexian relative to apixaban derived from stratified Cox-regression model and A is the non-inferiority margin (NI margin). The alternate hypothesis (H₁) is that milvexian is non-inferior to apixaban while the null hypothesis (H₀) is that milvexian is inferior to apixaban.

A key aspect of developing adequate sample size for this trial is arriving at the appropriate NI margin (Δ) from historical studies, whilst aligning for constancy of the primary endpoint (stroke+SE) and study population. The estimation is based on primary efficacy analysis in the Full-Analysis Set up to the global targeted endpoint date (GTED). The assumptions and parameters are: (1) event-driven study with variable treatment duration; (2) power of at least 90%: composite of stroke and non-CNS systemic embolism; (3) one-sided significance level of 0.025; (4) non-inferiority margin (4) for the composite of stroke and non-CNS systemic embolism of 1.37 (additional detail can be found in the section below.); (5) apixaban treatment group event rate of 1.33% per year for primary efficacy composite outcome of stroke and non-CNS systemic embolism; (6) Hazard Ratio=1.00 for milvexian compared to apixaban; (7) non-uniform enrollment of 140 participants per month (Months 1-6); 280 participants per month (Months 7-12), 420 participants per month (Months 13-43). To achieve 90% power to test for non-inferiority using a margin of 1.37 and the additional assumptions listed above, 430 composite of stroke and non-CNS systemic embolization events are needed, and a sample size of approximately 15,500 participants (7,750 per arm) is required.

The primary objective of the study is to establish that milvexian is non-inferior to apixaban in reducing the risk of stroke and non-CNS systemic embolism. The stratified Cox-proportional Hazard model is used for calculating the Hazard ratio (HZ) of milvexian 100 mg versus apixaban 5 mg twice daily (2.5 mg twice daily for participants with at least 2 of the following characteristics: age ≥80 years, body weight ≤60 kg, or serum creatinine ≥1.5 mg/dL), along with the 2-sided 95% CI. If the upper bound of the 2-sided 95% confidence interval of the HR of milvexian versus apixaban is less than 1.37 (non-inferiority margin), the lower bound of the 2-sided 95% confidence interval of the HR of milvexian versus apixaban is less than 1.00 (inferiority boundary), and the 1-sided non-inferiority p-value from the stratified Cox proportional hazard model is <0.025, then non-inferiority of the milvexian will be declared. If the upper bound of the 2-sided 95% confidence interval of the HR of milvexian versus apixaban is less than 1.00, then superiority of milvexian will be declared.

In some embodiments, the methods of the present invention result in a patient's hazard ratio (HR) (comparing risk in treated group versus risk in placebo group per industry standards) for one or more of particular vascular thrombotic events or outcome, for example, ischemic stroke, non-CNS systemic embolism, all-cause death, myocardial infarction, stroke, or non-CNS embolism, acute limb ischemia, or urgent hospitalization for vascular cause of ischemic nature (including DVT and PE) in a human patient with atrial fibrillation or flutter, in a range of from about 0.5 to about 1.1, or any amount or range therein, preferably in the range of from about 0.6 to about 0.9, more preferably in a range of from about 0.75 to about 0.9, more preferably in a range of from about 0.65 to about 0.85, for example, less than about 1.0, 0.99, 0.98, 0.97, 0.96, 0.95, 0.94, 0.93, 0.92, 0.91, 0.90, 0.89, 0.88, 0.87, 0.86, 0.85, 0.84, 0.83, 0.82, 0.81, 0.80, 0.79, 0.78, 0.77, 0.76, 0.75, 0.74, 0.73, 0.72, 0.71, 0.70, 0.69, 0.68, 0.67, 0.66, 0.65, 0.64, 0.63, 0.62, 0.61, 0.60, 0.59, 0.58, 0.57, 0.56, 0.55, 0.54, 0.53, 0.52, 0.51 or 0.50 or any range defined by any two of the preceding values.

In certain embodiments, one or more improvements provided by the methods of the present invention (e.g. reduction in the risk of stroke and systemic embolism (SE), reduction in the predicted severity of an adverse cardiovascular event, decrease in the predicted mortality from an adverse cardio-vascular event, decrease in the progression of cardiovascular disease in a patient, increase in the predicted life expectancy of the patient, or increase in the predicted time period until next occurrence of an adverse cardiovascular event, the increase in the effectiveness of a cardiovascular intervention in a patient), may continue for a period of time after the discontinuation of the administration of milvexian. In certain embodiments, this period of time is, or is at least, about 1, 2, 3, 4, 5, or 6 months, or 0.5, 1, 2, 3, 4, or 5 years, or between 1-6 months, 1 month to 1 year, 4 months to 2 years, or 6 months to 5 years.

In certain embodiments of the present invention, one or more improvements provided by the methods of the present invention (e.g. reduction in the risk of one or more stroke and SE, reduction in the predicted severity of an adverse cardiovascular event, decrease in the predicted mortality from an adverse cardiovascular event, decrease in the progression of cardiovascular disease in a patient, increase in the predicted life expectancy of the patient, or increase in the predicted time period until next occurrence of an adverse cardiovascular event, the increase in the effectiveness of a cardiovascular intervention in a patient, or favorable modulation in a diagnostic indicator predictive of a adverse cardiovascular event), is seen in a treated patient population as compared to a control population, for example between patients receiving milvexian and patients receiving apixaban.

In some aspects of the methods of the disclosure, the patient is administered a pharmaceutical composition comprising about 50 mg, about 75.0 mg, about 87.5 mg, or about 100 mg, of milvexian (or a pharmaceutically acceptable salt or solvate thereof, on a milvexian basis), wherein the composition is administered twice daily.

In some aspects of the methods of the disclosure, the patient is administered a pharmaceutical composition comprising about 50 mg, or about 100 mg, of milvexian (or a pharmaceutically acceptable salt or solvate thereof, on a milvexian basis), wherein the composition is administered twice daily.

In some aspects of the methods of the disclosure, the patient is administered a pharmaceutical composition comprising about 50 mg, of milvexian (or a pharmaceutically acceptable salt or solvate thereof, on a milvexian basis), wherein the composition is administered twice daily.

In some aspects of the methods of the disclosure, the patient is administered a pharmaceutical composition comprising about 75.0 mg, of milvexian (or a pharmaceutically acceptable salt or solvate thereof, on a milvexian basis), wherein the composition is administered twice daily.

In some aspects of the methods of the disclosure, the patient is administered a pharmaceutical composition comprising about 87.5 mg, of milvexian (or a pharmaceutically acceptable salt or solvate thereof, on a milvexian basis), wherein the composition is administered twice daily.

In some aspects of the methods of the disclosure, the patient is administered a pharmaceutical composition comprising about 100 mg, of milvexian (or a pharmaceutically acceptable salt or solvate thereof, on a milvexian basis), wherein the composition is administered twice daily.

In embodiments of the disclosed methods in which the pharmaceutical composition comprises a solvate or a pharmaceutically acceptable salt of milvexian, then the amount specified is on a milvexian basis. That is, an amount of the solvate or the pharmaceutically acceptable salt in the pharmaceutical composition contains the specified amount of milvexian. For example, a pharmaceutical composition comprising 100 mg of milvexian (or pharmaceutically acceptable salt or solvate thereof) refers to a pharmaceutical composition comprising either about 100 mg of milvexian, or an amount of a pharmaceutically acceptable salt or solvate of milvexian equivalent to about 100 mg of milvexian. In some embodiments, the pharmaceutically acceptable solvate of milvexian is a hydrate including monohydrate, hemi-hydrate, or a mixture of monohydrate and hemi-hydrate. In some embodiments, the pharmaceutically acceptable solvate of milvexian is a channel hydrate.

In some embodiments the patient is administered a pharmaceutical composition comprising about 50 mg of milvexian, wherein the composition is administered twice daily.

In some embodiments the patient is administered a pharmaceutical composition comprising about 50 mg (on a milvexian basis) of a pharmaceutically acceptable salt or solvate of milvexian, wherein the composition is administered twice daily.

In some embodiments the patient is administered a pharmaceutical composition comprising about 50 mg (on a milvexian basis) of a pharmaceutically acceptable salt of milvexian, wherein the composition is administered twice daily.

In some embodiments, the patient is administered a pharmaceutical composition comprising about 50 mg (on a milvexian basis) of a pharmaceutically acceptable solvate of milvexian, wherein the composition is administered twice daily. In some embodiments, the pharmaceutically acceptable solvate of milvexian is a hydrate including monohydrate, hemi-hydrate, or a mixture of monohydrate and hemi-hydrate. In some embodiments, the pharmaceutically acceptable solvate of milvexian is a channel hydrate.

In some embodiments the patient is administered a pharmaceutical composition comprising about 100 mg of milvexian, wherein the composition is administered twice daily.

In some embodiments the patient is administered a pharmaceutical composition comprising about 100 mg (on a milvexian basis) of a pharmaceutically acceptable salt or solvate of milvexian, wherein the composition is administered twice daily.

In some embodiments the patient is administered a pharmaceutical composition comprising about 100 mg (on a milvexian basis) of a pharmaceutically acceptable salt of milvexian, wherein the composition is administered twice daily.

In some embodiments the patient is administered a pharmaceutical composition comprising about 100 mg (on a milvexian basis) of a pharmaceutically acceptable solvate of milvexian, wherein the composition is administered twice daily. In some embodiments, the pharmaceutically acceptable solvate of milvexian is a hydrate including monohydrate, hemi-hydrate, or a mixture of monohydrate and hemi-hydrate. In some embodiments, the pharmaceutically acceptable solvate of milvexian is a channel hydrate.

In some aspects of the methods of the disclosure, the human patient is administered a pharmaceutical composition comprising about 50 mg to about 200 mg of milvexian (or a pharmaceutically acceptable salt or solvate thereof, on a milvexian basis) twice daily.

In some embodiments the patient is administered a pharmaceutical composition comprising about 50 mg to about 200 mg of milvexian, wherein the composition is administered twice daily.

In some embodiments the patient is administered a pharmaceutical composition comprising about 50 mg to about 200 mg (on a milvexian basis) of a pharmaceutically acceptable salt or solvate of milvexian, wherein the composition is administered twice daily.

In some embodiments the patient is administered a pharmaceutical composition comprising about 50 mg to about 200 mg (on a milvexian basis) of a pharmaceutically acceptable salt of milvexian, wherein the composition is administered twice daily.

In some embodiments the patient is administered a pharmaceutical composition comprising about 50 mg to about 200 mg (on a milvexian basis) of a pharmaceutically acceptable solvate of milvexian, wherein the composition is administered twice daily. In some embodiments, the pharmaceutically acceptable solvate of milvexian is a hydrate including monohydrate, hemi-hydrate, or a mixture of monohydrate and hemi-hydrate. In some embodiments, the pharmaceutically acceptable solvate of milvexian is a channel hydrate.

In some embodiments the patient is administered a pharmaceutical composition comprising about 50 mg of milvexian, wherein the composition is administered twice daily.

In some embodiments the patient is administered a pharmaceutical composition comprising about 50 mg (on a milvexian basis) of a pharmaceutically acceptable salt or solvate of milvexian, wherein the composition is administered twice daily.

In some embodiments the patient is administered a pharmaceutical composition comprising about 50 mg (on a milvexian basis) of a pharmaceutically acceptable salt of milvexian, wherein the composition is administered twice daily.

In some embodiments the patient is administered a pharmaceutical composition comprising about 50 mg (on a milvexian basis) of a pharmaceutically acceptable solvate of milvexian, wherein the composition is administered twice daily.

In some embodiments the patient is administered a pharmaceutical composition comprising about 75.0 mg of milvexian, wherein the composition is administered twice daily.

In some embodiments the patient is administered a pharmaceutical composition comprising about 75.0 mg (on a milvexian basis) of a pharmaceutically acceptable salt or solvate of milvexian, wherein the composition is administered twice daily.

In some embodiments the patient is administered a pharmaceutical composition comprising about 75.0 mg (on a milvexian basis) of a pharmaceutically acceptable salt of milvexian, wherein the composition is administered twice daily.

In some embodiments the patient is administered a pharmaceutical composition comprising about 75.0 mg (on a milvexian basis) of a pharmaceutically acceptable solvate of milvexian, wherein the composition is administered twice daily. In some embodiments, the pharmaceutically acceptable solvate of milvexian is a hydrate including monohydrate, hemi-hydrate, or a mixture of monohydrate and hemi-hydrate. In some embodiments, the pharmaceutically acceptable solvate of milvexian is a channel hydrate.

In some embodiments the patient is administered a pharmaceutical composition comprising about 87.5 mg of milvexian, wherein the composition is administered twice daily.

In some embodiments the patient is administered a pharmaceutical composition comprising about 87.5 mg (on a milvexian basis) of a pharmaceutically acceptable salt or solvate of milvexian, wherein the composition is administered twice daily.

In some embodiments the patient is administered a pharmaceutical composition comprising about 87.5 mg (on a milvexian basis) of a pharmaceutically acceptable salt of milvexian, wherein the composition is administered twice daily.

In some embodiments the patient is administered a pharmaceutical composition comprising about 87.5 mg (on a milvexian basis) of a pharmaceutically acceptable solvate of milvexian, wherein the composition is administered twice daily. In some embodiments, the pharmaceutically acceptable solvate of milvexian is a hydrate including monohydrate, hemi-hydrate, or a mixture of monohydrate and hemi-hydrate. In some embodiments, the pharmaceutically acceptable solvate of milvexian is a channel hydrate.

In some aspects of the methods of the disclosure in which the human patient is administered the pharmaceutical composition comprising milvexian (or a pharmaceutically acceptable salt or solvate thereof, on a milvexian basis) twice daily, one of the two administrations is made in the morning, and the other of the two administrations is made in the evening.

In some embodiments, each of the morning administration and the evening administration is made at the same time each day.

In some aspects of the methods of the disclosure, the pharmaceutical composition comprising milvexian (or a pharmaceutically acceptable salt or solvate thereof, on a milvexian basis) is administered twice daily for at least 13 weeks.

In some embodiments, the pharmaceutical composition comprising milvexian (or a pharmaceutically acceptable salt or solvate thereof, on a milvexian basis) is administered twice daily for 13 weeks.

In other embodiments, the pharmaceutical composition comprising milvexian (or a pharmaceutically acceptable salt or solvate thereof, on a milvexian basis) is administered twice daily for more than 13 weeks.

In other embodiments, the pharmaceutical composition comprising milvexian (or a pharmaceutically acceptable salt or solvate thereof, on a milvexian basis) is administered twice daily for at least 26 weeks, at least 52 weeks, at least 78 weeks, at least 104 weeks, at least 130 weeks, at least 156 weeks, at least 182 weeks, or at least 208 weeks.

In some embodiments, the pharmaceutical composition comprising milvexian (or a pharmaceutically acceptable salt or solvate thereof, on a milvexian basis) is administered twice daily chronically, i.e., indefinitely.

In some aspects of the methods of the disclosure, the pharmaceutical composition comprising milvexian (or a pharmaceutically acceptable salt or solvate thereof) is a solid oral pharmaceutical composition comprising milvexian (or a pharmaceutically acceptable salt or solvate thereof) and one or more pharmaceutically acceptable excipients such as binder, filler, lubricant, disintegrant, and glidant.

In some embodiments, the solid oral pharmaceutical composition comprises a spray-dried amorphous solid dispersion (SDP) consisting essentially of milvexian free form and a pH-dependent enterosoluble polymer.

In some embodiments, the SDP comprises the milvexian free form and the pH-dependent enterosoluble polymer in a weight ratio of 3:1 (milvexian:polymer).

In some embodiments, the pH-dependent enterosoluble polymer is cellulose acetate trimellitate (CAT), cellulose acetate phthalate (CAP), Hydroxypropyl methylcellulose phthalate (HPMCP), hydroxypropyl methylcellulose ES grade (HPMC ES), hydroxypropyl methyl cellulose acetate succinate (HPMC-AS) LF, LG, MF, MG or HF Grades such as Aqoat®, Polyvinyl acetate phthalate (PVAP) such as Sureteric® and Opadry® and Shellac resins such as SSB® Aquagold, or polyvinylpyrrolidone (PVP).

In some embodiments, the pH-dependent enterosoluble polymer is hydroxypropyl methyl cellulose-AS MG.

In some embodiments, the SDP is a spray-dried amorphous solid dispersion prepared according to the composition and method described in WO 2020210629. This SDP consists essentially of milvexian free form and hypromellose acetate succinate (HPMCAS-MG) in a weight ratio of 3:1 (milvexian:HPMCAS-MG).

In other embodiments, the solid oral pharmaceutical composition further comprises a binder, such as, for example, microcrystalline cellulose (MCC), silicified microcrystalline cellulose (SMCC), or a combination thereof.

In some embodiments, the solid oral pharmaceutical composition further comprises a filler, such as, for example, lactose monohydrate.

In some embodiments, the solid oral pharmaceutical composition further comprises a disintegrant.

In some embodiments, the solid oral pharmaceutical composition further comprises a lubricant.

In some embodiments, the solid oral pharmaceutical composition is a tablet.

In some embodiments, the solid oral pharmaceutical composition is a direct compression tablet.

In other embodiments, the solid oral pharmaceutical composition is a roller compaction tablet.

In some embodiments, the solid oral pharmaceutical composition is a film-coated tablet.

In some embodiments, the film coating comprises polyvinyl alcohol, titanium dioxide, polyethylene glycol-polyvinyl alcohol graft copolymer, and talc.

In other embodiments, the film coating comprises polyethylene glycol-polyvinyl alcohol graft copolymer.

In some embodiments, the film coating comprises polyvinyl alcohol, colorant, macrogol (PEG) polyvinyl alcohol grafted copolymer, and talc.

In some embodiments, the film coating comprises polyvinyl alcohol, iron oxide, macrogol (PEG) polyvinyl alcohol grafted copolymer, and talc.

In some embodiments, the film coating comprises OpadryQX 321A220063 Yellow, a film coating material that comprises polyvinyl alcohol, iron oxide, macrogol (PEG) polyvinyl alcohol grafted copolymer, and talc.

In some embodiments, the solid oral pharmaceutical composition is an immediate release tablet.

In some embodiments, the solid oral pharmaceutical composition is a film-coated immediate release tablet.

In some embodiments, the solid oral pharmaceutical composition is a film-coated immediate release tablet rapidly dispersible in aqueous media (e.g., water or applesauce).

In some embodiments, the solid oral pharmaceutical composition is a tablet having the composition and/or properties shown in Table A:

TABLE A
Film-coated Tablet of Milvexian at 100 mg or 25 mg Strength

| | Amount | | |
|---|---|---|---|
| | | Amount | Amount |
| | | per dose | per dose |
| Component | wt. % | (25 mg) | (100 mg) |
| SDP^{b} | 22.22 | 33.33 | 133.3 |
| Silicified Microcrystalline cellulose | 43.07 | 64.6 | 258.4 |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 Yellow^{c} | 3.0 | 4.5 | 18.0 |
| | | 154.5 | 618 |
| Tablet hardness | | Average 60N to | Average 140N to |
| | | 120N (Ph. | 220N (Ph. |
| | | Eur. 2.9.8), | Eur. 2.9.8), |
| | | preferably 90N | preferably 180N |
| Tablet friability | | <0.5% | <0.5% |
| Disintegration time in water | | 13 | 18 |
| (seconds) | | | |

^{b}SDP: spray-dried amorphous solid dispersion prepared according to the composition and method described in WO 2020210629. SDP consists essentially of milvexian free form and hypromellose acetate succinate (HPMCAS-MG) in a weight ratio of 3:1 (milvexian:HPMCAS-MG).
^{c}OpadryQX 321A220063 Yellow: a film coating material comprises polyvinyl alcohol, iron oxide, macrogol (PEG) polyvinyl alcohol grafted copolymer, and talc.

In some embodiments in which the solid oral pharmaceutical composition is a tablet, the tablet has a disintegration time in water of less than 20 seconds at 37° C.

As used herein, the term “disintegration time” refers to the time required for the tablet to break into particles under a given set of conditions. In some embodiments, the disintegration time is determined using the apparatus described in Eur. Ph. (PTZ-E Pharma Test, Hainburg, Germany), in distilled water at 37° C. using disks.

In other embodiments in which the solid oral pharmaceutical composition is a tablet, the tablet has a disintegration time in water of less than 15 seconds at 37° C.

In other embodiments in which the solid oral pharmaceutical composition is a tablet, the tablet has a disintegration time in water of less than 10 seconds at 37° C.

In some embodiments, the solid oral pharmaceutical composition is a capsule.

In some aspects of the methods of the disclosure, the human patient to who the composition comprising milvexian (or a pharmaceutically acceptable salt or solvate thereof) is administered, is unable to swallow a tablet dosage form.

In some embodiments of the disclosed methods, the solid oral pharmaceutical composition is dispersed in an aqueous medium to form an aqueous dispersion for administration.

In some embodiments of the disclosed methods, the solid oral pharmaceutical composition is a tablet which is dispersed in an aqueous medium to form an aqueous dispersion for administration.

In those embodiments in which the solid oral pharmaceutical composition is dispersed in an aqueous medium to form an aqueous dispersion, the aqueous medium is water, saline, phosphate buffer, vegetable juice, or a fruit juice, including, for example, apple sauce.

In those embodiments in which the solid oral pharmaceutical composition is a tablet which is dispersed in an aqueous medium to form an aqueous dispersion, the aqueous medium is water, saline, phosphate buffer, vegetable juice, or a fruit juice, including, for example, apple sauce.

In some embodiments of the disclosed methods in which the solid oral pharmaceutical composition is dispersed in an aqueous medium to form an aqueous dispersion for administration, the aqueous dispersion is administered to the human patient via a nasogastric tube or spoon.

In some embodiments of the disclosed methods in which the solid oral pharmaceutical composition is a tablet which is dispersed in an aqueous medium to form an aqueous dispersion for administration, the aqueous dispersion is administered to the human patient via a nasogastric tube or spoon.

In some embodiments of the disclosed methods, oral administration of the solid oral pharmaceutical composition results in a milvexian plasma half-life ranging from about 13 hours to about 16 hours. In some embodiments of the disclosed methods, oral administration of the pharmaceutical composition comprising milvexian (or a pharmaceutically acceptable salt or solvate thereof) results in a milvexian plasma half-life ranging from about 13 hours to about 16 hours during repeat dosing. “Half-life (t_{1/2}),” as used herein, refers to apparent terminal elimination half-life having a mean value calculated using formula t_{1/2}=logₑ2/λz, where λz is the apparent terminal elimination rate-constant estimated by linear regression using the terminal log-linear phase of the logarithmic (log) transformed concentration vs time data. In some embodiments, half-life (t_{1/2}) has a mean value ranges from about 8 hours to about 16 hours. In some embodiments, half-life (t_{1/2}) has a mean value ranges from about 11 hours to about 16 hours. In a preferred embodiment, half-life (t_{1/2}) has a mean value ranges from about 13 hours to about 16 hours.

In some embodiments of the disclosed methods, administration of the solid oral pharmaceutical composition comprising milvexian (or a pharmaceutically acceptable salt or solvate thereof) to the human patient results in milvexian plasma concentrations reaching steady-state at about 3 days to 6 days. As used herein, the term “steady-state” refers to steady-state plasma concentrations as defined by regulatory agencies such as the U.S. Food & Drug Administration (FDA) and the European Medicines Agency (EMA).

In some embodiments of the disclosed methods, administration of the solid oral pharmaceutical composition comprising milvexian (or a pharmaceutically acceptable salt or solvate thereof) to the human patient results in milvexian plasma concentrations reaching steady-state in about 4 days.

In some embodiments of the disclosed methods, administration of the solid oral pharmaceutical composition comprising milvexian (or a pharmaceutically acceptable salt or solvate thereof) to the human patient results in milvexian plasma concentrations reaching steady-state in about 5 days.

In some embodiments of the disclosed methods, administration of the solid oral pharmaceutical composition comprising milvexian (or a pharmaceutically acceptable salt or solvate thereof) to the human patient results in milvexian plasma concentrations reaching steady-state in about 6 days.

In some embodiments, milvexian has a terminal plasma half-life (t_{1/2}) ranging from about 13 hours to about 16 hours.

In some embodiments, administration of milvexian 100 mg BID as monotherapy to the human patient results in milvexian plasma concentrations reaching steady-state characterized by one or more of the following: (i) a steady state Cₘₐₓ ranging from about 1194 ng/mL to about 2326 ng/mL, (ii) a steady state mean (std) Cₘₐₓ of 1760 (566) ng/ml, (iii) a steady state AUC₀₋₂₄ ranging from about 23300 ng*h/mL to about 49100 ng*h/mL, or (iv) a steady state mean (std) AUC₀₋₂₄ of 36200 (12900) ng*h/mL.

In other embodiments, the steady state after the administration of milvexian 100 mg BID as monotherapy to the human patient is characterized by Cₘₐₓ ranging from about 1194 ng/ml to about 2326 ng/mL.

In other embodiments, the steady state after the administration of milvexian 100 mg BID as monotherapy to the human patient is characterized by mean (std) Cₘₐₓ of 1760 (566) ng/mL.

In other embodiments, the steady state after the administration of milvexian 100 mg BID as monotherapy to the human patient is characterized by AUC₀₋₂₄ ranging from about 23300 ng*h/mL to about 49100 ng*h/mL.

In other embodiments, the steady state after the administration of milvexian 100 mg BID as monotherapy to the human patient is characterized by mean (std) AUC of 36200 (12900) ng*h/mL.

In some embodiments, administration of milvexian 100 mg BID on top of standard care antiplatelet therapy to the human patient results in milvexian plasma concentrations reaching steady-state characterized by one or more of the following: (i) a steady state Cₘₐₓ ranging from about 1134 ng/ml to about 2206 ng/ml, (ii) a steady state Cₘₐₓ at mean (std) of 1670 (536) ng/mL, (iii) a steady state AUC₀₋₂₄ ranging from about 22900 ng*h/mL to about 47100 ng*h/mL, or (iv) a steady state AUC₀₋₂₄ at mean (std) of 35000 (12100) ng*h/mL.

In other embodiments, the steady state after the administration of milvexian 100 mg BID on top of standard care antiplatelet therapy to the human patient is characterized by Cₘₐₓ ranging from about 1134 ng/ml to about 2206 ng/mL.

In other embodiments, the steady state after the administration of milvexian 100 mg BID on top of standard care antiplatelet therapy to the human patient is characterized by mean (std) Cₘₐₓ of 1670 (536) ng/mL.

In other embodiments, the steady state after the administration of milvexian 100 mg BID on top of standard care antiplatelet therapy to the human patient is characterized by AUC₀₋₂₄ ranging from about 22900 ng*h/mL to about 47100 ng*h/mL

In other embodiments, the steady state after the administration of milvexian 100 mg BID on top of standard care antiplatelet therapy to the human patient is characterized by mean (std) AUC of 35000 (12100) ng*h/mL.

In some embodiments of the disclosed methods, the solid oral pharmaceutical composition comprising milvexian (or a pharmaceutically acceptable salt or solvate thereof) is administered without regard to the timing of food intake.

In some aspects of the methods of the disclosure, the administration of the pharmaceutical composition comprising milvexian (or a pharmaceutically acceptable salt or solvate thereof) reduces FXI clotting activity in the patient by an amount ranging from about 27% to about 64% relative to baseline. Methods of determining FXI clotting activity are described herein. As used here “baseline” refers to the patient's FXI clotting activity prior to the administration of the composition comprising milvexian (or a pharmaceutically acceptable salt or solvate thereof).

In some embodiments, the administration of the pharmaceutical composition comprising milvexian (or a pharmaceutically acceptable salt or solvate thereof) reduces FXI clotting activity in the patient by an amount of about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, or about 64%, relative to baseline.

In some embodiments of the disclosed methods, the administration results in a prolongation of activated partial thromboplastin time (aPTT) in the patient with a ratio to baseline of 2.1 to 2.6, such as, for example, 2.1, 2.2, 2.3, 2.4, 2.5, or 2.6.

In some aspects of the disclosed methods, the administration does not result in a statistically significant increase in major bleeding complications. In these aspects, “statistically significant increase” refers to an increase from the patient's baseline major bleeding, i.e., the patient's major bleeding before being administered milvexian (or a pharmaceutically acceptable salt or solvate thereof).

In some embodiments, the administration does not result in a statistically significant increase in major bleeding complications as assessed under ISTH criteria.

In some embodiments, the administration does not result in a statistically significant increase in major bleeding complications as assessed by CRNM bleeding criteria.

In some embodiments, the administration does not result in a statistically significant increase in major bleeding complications as assessed by ISTH CRNM bleeding criteria.

In some embodiments, the administration does not result in a statistically significant increase in major bleeding complications as assessed by ISTH major or CRNM bleeding criteria.

In some embodiments, the administration does not result in a statistically significant increase in major bleeding complications as assessed by GUSTO, BARC, or TIMI criteria.

In some embodiments of the methods of the disclosure, the administration does not result in a statistically significant increase in GUSTO severe or life-threatening bleeding.

In some embodiments of the methods of the disclosure, the administration does not result in a statistically significant increase in major bleeding complications as assessed by BARC 3c and 5 categories.

In some embodiments of the methods of the disclosure, the administration does not result in a statistically significant increase in major bleeding complications as assessed by BARC 3b, 3c, and 5 categories.

In some embodiments of the methods of the disclosure, the administration does not result in a statistically significant increase in clinically relevant nonmajor bleeding as assessed by TIMI major bleeding.

In some embodiments of the methods of the disclosure, the administration does not result in a statistically significant increase in clinically relevant nonmajor bleeding as assessed by TIMI major bleeding or TIMI minor bleeding.

In some aspects, the disclosure provides methods of preventing one or more of stroke and non-central nervous system (CNS) systemic embolism events in a patient with a history of atrial fibrillation or flutter, wherein the method comprises administering to the patient an immediate release film coated tablet comprising 50 mg or 100 mg of milvexian (or a pharmaceutically acceptable salt thereof, on a milvexian basis) and one or more pharmaceutically acceptable excipients, wherein the immediate release film-coated tablet is administered twice a day with or without a meal, wherein the patient is at least 18 years old; wherein the patient further has one or more of Category (A) risk factors selected from (i) age greater than or equal to 75 years or (ii) history of ischemic stroke or silent brain infarct; or two or more of concomitant or comorbid Category (B) cardiovascular risk factor selected from the group consisting of: (i) age between 65 and 74 years; (ii) hypertension; (iii) diabetes mellitus; (iv) history of coronary artery disease (CAD) (including post myocardial infarction or acute coronary syndrome event with or without percutaneous coronary intervention (PCI)) or peripheral artery disease (PAD); and (v) history of heart failure. In some embodiments, heart failure is defined as either: (a) history of hospitalization with HF as the primary cause (regardless of ejection fraction) or (b) left ventricular ejection fraction (LVEF) of <40% (most recent and assessed <1 year) regardless of history of heart failure hospitalization).

In other aspects, the disclosure provides methods of preventing one or more adverse cardiovascular events in a patient with atrial fibrillation or flutter, wherein each event is selected from the group consisting of cardiovascular death, myocardial infarction, stroke, and non-central nervous system (CNS) systemic embolism, said method comprising administering to the patient an immediate release film-coated tablet comprising 50 mg or 100 mg of milvexian, or a pharmaceutically acceptable salt thereof, and one or more pharmaceutically acceptable excipient, wherein the immediate release film-coated tablet is administered twice a day with or without a meal, wherein the patient is at least 18 years old; wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of: (i) age between 65 and 74 years; (ii) hypertension; (iii) diabetes mellitus; (iv) history of coronary artery disease (CAD) (including post myocardial infarction or acute coronary syndrome event with or without percutaneous coronary intervention (PCI)) or peripheral artery disease (PAD); and (v) history of heart failure. In some embodiments, heart failure is defined as either: (a) history of hospitalization with HF as the primary cause (regardless of ejection fraction) or (b) left ventricular ejection fraction (LVEF) of <40% (most recent and assessed <1 year) regardless of history of heart failure hospitalization).

In some embodiments of such methods, the adverse cardiovascular event is cardiovascular death.

Administration of milvexian according to the disclosure is generally safe and well tolerated. In some aspects, administration of milvexian, at a dose of 25 mg, 50 mg, or 100 mg results in no clinically significant QTc interval prolongation. For example, administration of milvexian, at a dose of 25 mg, 50 mg, or 100 mg does not result in a ΔΔQTc of 10 msec or longer. In some aspects, administration of milvexian, at a dose of 25 mg, 50 mg, or 100 mg results in a ΔΔQTc of less than 10 msec. In some aspects, administration of milvexian, at a dose of 25 mg, 50 mg, or 100 mg results in ΔΔQTc of less than 9 msec. In some aspects, administration of milvexian, at a dose of 25 mg, 50 mg, or 100 mg results in a ΔΔQTc of less than 8 msec. In some aspects, administration of milvexian, at a dose of 25 mg, 50 mg, or 100 mg results in a ΔΔQTc in QTc of less than 7 msec. In some aspects, administration of milvexian, at a dose of 25 mg, 50 mg, or 100 mg results in a ΔΔQTc of less than 6 msec. In some aspects, administration of milvexian, at a dose of 25 mg, 50 mg, or 100 mg results in a ΔΔQTc of less than 5 msec. In some aspects, administration of milvexian, at a dose of 25 mg, 50 mg, or 100 mg results in a ΔΔQTc of less than 4 msec.

In some aspects, administration of milvexian at a dose of 50 mg results in no clinically significant QTc interval prolongation. In some aspects, administration of milvexian, at a dose of 50 mg, results in a ΔΔQTc of less than 10 msec. In some aspects, administration of milvexian, at a dose of 50 mg, results in a ΔΔQTc of less than 9 msec. In some aspects, administration of milvexian, at a dose of 50 mg, results in a ΔΔQTc of less than 8 msec. In some aspects, administration of milvexian, at a dose of 50 mg, results in a ΔΔQTc of less than 7 msec. In some aspects, administration of milvexian, at a dose of 50 mg, results in a ΔΔQTc of less than 6 msec. In some aspects, administration of milvexian, at a dose of 50 mg, results in a ΔΔQTc of less than 5 msec. In some aspects, administration of milvexian, at a dose of 50 mg, results in a ΔΔQTc of less than 4 msec.

In some aspects, administration of milvexian at a dose of 100 mg results in no clinically significant QTc interval prolongation. In some aspects, administration of milvexian, at a dose of 100 mg, results in a ΔΔQTc of less than 10 msec. In some aspects, administration of milvexian, at a dose of 100 mg, results in a ΔΔQTc of less than 9 msec. In some aspects, administration of milvexian, at a dose of 100 mg, results in a ΔΔQTc of less than 8 msec. In some aspects, administration of milvexian, at a dose of 100 mg, results in a ΔΔQTc of less than 7 msec. In some aspects, administration of milvexian, at a dose of 100 mg, results in a ΔΔQTc of less than 6 msec. In some aspects, administration of milvexian, at a dose of 100 mg, results in a ΔΔQTc of less than 5 msec. In some aspects, administration of milvexian, at a dose of 100 mg, results in a ΔΔQTc of less than 4 msec.

The disclosure is directed to a method of preventing stroke and non-CNS systemic embolism in an adult patient with atrial fibrillation (AF), comprising administering to the patient 100 mg BID milvexian. The disclosure is directed to methods of preventing stroke and non-CNS systemic embolism in adult patients with atrial fibrillation (AF), comprising administering 100.0 mg BID milvexian.

### Aspects

It will be understood that references herein to methods of using milvexian or compositions comprising milvexian for treating or preventing the conditions of the disclosure should also be interpreted as references to: (i) the milvexian or compositions comprising milvexian for use in methods of treating or preventing the conditions of the disclosure; and/or (ii) the use of milvexian or compositions comprising milvexian in the manufacture of a medicament for treating or preventing the conditions of the disclosure.

The disclosure is also directed to the following aspects.

### Aspect Set #1

Aspect 1. A method for preventing one or more of stroke and non-central nervous system (CNS) systemic embolism events in a human patient with atrial fibrillation, wherein the method comprises administering to the human patient a pharmaceutical composition comprising about 100 mg of milvexian (or a pharmaceutically acceptable salt or solvate thereof, on a milvexian basis); wherein the pharmaceutical composition is administered twice daily.
Aspect 2. A method for preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about about 100 mg of milvexian (or a pharmaceutically acceptable salt or solvate thereof, on a milvexian basis); wherein the composition is administered twice daily.
Aspect 3. The method of aspect 2, wherein the adverse cardiovascular event is cardiovascular death.
Aspect 4. The method of any one of aspects 1-3, wherein the patient has atrial fibrillation, and also has concomitant coronary artery disease (CAD) and/or peripheral artery disease (PAD).
Aspect 5. The method of any one of aspects 1-4, wherein the pharmaceutical composition is a solid oral pharmaceutical composition comprising milvexian (or a pharmaceutically acceptable salt or solvate thereof) and one or more pharmaceutically acceptable excipients.
Aspect 6. The method of aspect 5, wherein the solid oral pharmaceutical composition is an immediate release tablet.
Aspect 7. The method of aspect 6, wherein the immediate release tablet has a disintegration time in water of less than 20 seconds at 37° C.
Aspect 8. The method of any one of aspects 5-7, wherein the administration results in a milvexian plasma half-life ranging from about 13 hours to about 16 hours.
Aspect 9. The method of any one of aspects 5-7, wherein the administration results in a milvexian plasma half-life ranging from about 13 hours to about 16 hours during repeat dosing.
Aspect 10. The method of any one of the preceding aspects, wherein the administration results in milvexian plasma concentrations reaching steady-state at about 4 days to 6 days.
Aspect 11. The method of any one of the preceding aspects, wherein the pharmaceutical composition comprising milvexian (or a pharmaceutically acceptable salt or solvate thereof) is administered without regard to the timing of food intake.
Aspect 12. The method of any one of the preceding aspects, wherein the composition comprising milvexian (or a pharmaceutically acceptable salt or solvate thereof) is administered twice daily for at least 13 weeks.
Aspect 13. The method of any one of the preceding aspects, wherein the composition comprising milvexian (or a pharmaceutically acceptable salt or solvate thereof) is administered once daily in the morning and once daily in the evening.
Aspect 14. The method of any one of the preceding aspects, wherein the morning administration is made at the same time each day and the evening administration is made at the same time each day.
Aspect 15. A method of preventing one or more of stroke and non-central nervous system (CNS) systemic embolism events in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 16. A method of preventing one or more adverse cardiovascular events in a patient with atrial fibrillation, wherein each event is selected from the group consisting of cardiovascular death, myocardial infarction, stroke, and non-central nervous system (CNS) systemic embolism said method comprising administering to the patient an immediate release film-coated tablet comprising 100 mg of milvexian, or a pharmaceutically acceptable salt thereof, and one or more pharmaceutically acceptable excipient,
   wherein the immediate release film-coated tablet is administered twice a day with or without a meal,
   wherein the patient is at least 18 years old;
   wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 17. The method of aspect 16, wherein the adverse cardiovascular event is stroke and non-central nervous system (CNS) systemic embolism.
Aspect 18. The method of any one of aspects 15-17, wherein the immediate release film-coated tablet has a disintegration time in water of less than 20 seconds at 37° C.
Aspect 19. The method of any one of aspects 15-18, wherein the administered milvexian has a plasma half-life of 13-16 hours.
Aspect 20. The method of any one of aspects 15-18, wherein the administered milvexian has a plasma half-life of 13-16 hours during repeat dosing.
Aspect 21. The method of any one of aspects 15-20, wherein the composition comprising milvexian (or a pharmaceutically acceptable salt or solvate thereof) is administered twice daily for at least 13 weeks.
Aspect 22. The method of any one of aspects 15-21, wherein the composition comprising milvexian (or a pharmaceutically acceptable salt or solvate thereof) is administered once daily in the morning and once daily in the evening.
Aspect 23. The method of aspect 22, wherein the morning administration is made at the same time each day and the evening administration is made at the same time each day.
Aspect 24. The method of any one of the preceding aspects, wherein the administration reduces FXI clotting activity in the patient by about 27% to about 64% relative to the baseline FXI clotting activity.
Aspect 25. The method of any one of the preceding aspects, wherein the administration results in a prolongation of activated partial thromboplastin time (aPTT) in the patient with a ratio to baseline of 2.1 to 2.6.
Aspect 26. A method for preventing stroke in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 27. A method for preventing one or more non-central nervous system (CNS) systemic embolism in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 28. A method for preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.
Aspect 29. The method of aspect 28, wherein the adverse cardiovascular event is cardiovascular death.
Aspect 30. The method of aspect 28, wherein the adverse cardiovascular event is myocardial infarction.
Aspect 31. The method of aspect 28, wherein the adverse cardiovascular event is stroke.
Aspect 32. The method of aspect 28, wherein the adverse cardiovascular event is non-central nervous system (CNS) systemic embolism.
Aspect 33. The method of any one of aspects 26-32, wherein the patient has atrial fibrillation, and also has concomitant coronary artery disease (CAD) and/or peripheral artery disease (PAD).
Aspect 34. The method of aspect 33, wherein the patient has atrial fibrillation, and also has concomitant coronary artery disease (CAD).
Aspect 35. The method of aspect 33, wherein the patient has atrial fibrillation, and also has concomitant peripheral artery disease (PAD).
Aspect 36. The method of any one of aspects 26-35, wherein the pharmaceutical composition is a solid oral pharmaceutical composition comprising milvexian (or a pharmaceutically acceptable salt or solvate thereof) and one or more pharmaceutically acceptable excipients.
Aspect 37. The method of aspect 36, wherein the solid oral pharmaceutical composition is an immediate release tablet.
Aspect 38. The method of aspect 37, wherein the immediate release tablet has a disintegration time in water of less than 20 seconds at 37° C.
Aspect 39. The method of any one of aspects 26-38, wherein the administration results in a milvexian plasma half-life ranging from about 13 hours to about 16 hours.
Aspect 40. The method of any one of aspects 26-39, wherein the administration results in a milvexian plasma half-life ranging from about 13 hours to about 16 hours during repeat dosing.
Aspect 41. The method of any one of aspects 26-40, wherein the administration results in milvexian plasma concentrations reaching steady-state at about 4 days to 6 days.
Aspect 42. The method of any one of aspects 26-41, wherein the pharmaceutical composition comprising milvexian (or a pharmaceutically acceptable salt or solvate thereof) is administered without regard to the timing of food intake.
Aspect 43. The method of any one aspects 26-42, wherein the patient is unable to swallow a tablet dosage form.
Aspect 44. The method of any one aspects 26-43, wherein the pharmaceutical composition is dispersed in an aqueous medium to form an aqueous dispersion.
Aspect 45. The method of aspect 44, wherein the aqueous medium is water, saline, phosphate buffer, vegetable juice, or fruit juice, including, for example, apple sauce.
Aspect 46. The method of aspect 44 or aspect 45, wherein the aqueous dispersion is administered to the human patient via a nasogastric tube or spoon.
Aspect 47. The method of any one of aspects 44-46, wherein the milvexian is administered orally as an aqueous dispersion by dispersing the pharmaceutical composition in an aqueous medium in less than 60 seconds at 37° C.
Aspect 48. The method of any one of any one of aspects 26-47, wherein the composition comprising milvexian (or a pharmaceutically acceptable salt or solvate thereof) is administered twice daily for at least 13 weeks.
Aspect 49. The method of any one of any one of aspects 26-48, wherein the composition comprising milvexian (or a pharmaceutically acceptable salt or solvate thereof) is administered once in the morning and once in the evening.
Aspect 50. The method of aspect 49, wherein the morning administration is made at the same time each day and the evening administration is made at the same time each day.
Aspect 51. The method of any one of aspects 26-50, wherein the administration reduces FXI clotting activity in the patient by about 27% to about 64% relative to the baseline FXI clotting activity.
Aspect 52. The method of any one of any one of aspects 26-51, wherein the administration results in a prolongation of activated partial thromboplastin time (aPTT) in the patient with a ratio to baseline of 2.1 to 2.6.
Aspect 53. A method of preventing stroke in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 54. A method of preventing non-central nervous system (CNS) systemic embolism events in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 55. The method of any one of aspects 53-54, wherein the patient has atrial fibrillation, and also has concomitant coronary artery disease (CAD) and/or peripheral artery disease (PAD).
Aspect 56. The method of aspect 55, wherein the patient has atrial fibrillation, and also has concomitant coronary artery disease (CAD).
Aspect 57. The method of aspect 55, wherein the patient has atrial fibrillation, and also has concomitant peripheral artery disease (PAD).
Aspect 58. The method of any one of aspects 53-57, wherein the immediate-release film-coated tablet has a disintegration time in water of less than 20 seconds at 37° C.
Aspect 59. The method of any one of aspects 53-58, wherein the administration results in a milvexian plasma half-life ranging from about 13 hours to about 16 hours.
Aspect 60. The method of any one of aspects 53-59, wherein the administration results in a milvexian plasma half-life ranging from about 13 hours to about 16 hours during repeat dosing.
Aspect 61. The method of any one of aspects 53-60, wherein the administration results in milvexian plasma concentrations reaching steady-state at about 4 days to 6 days.
Aspect 62. The method of any one of aspects 53-61, wherein the immediate-release film-coated tablet is administered without regard to the timing of food intake.
Aspect 63. The method of any one aspects 53-62, wherein the patient is unable to swallow a tablet dosage form.
Aspect 64. The method of any one aspects 53-63, wherein the immediate-release film-coated tablet is dispersed in an aqueous medium to form an aqueous dispersion.
Aspect 65. The method of aspect 64, wherein the aqueous medium is water, saline, phosphate buffer, vegetable juice, or fruit juice, including, for example, apple sauce.
Aspect 66. The method of aspect 65 or aspect 65, wherein the aqueous dispersion is administered to the human patient via a nasogastric tube or spoon.
Aspect 67. The method of any one of aspects 53-66, wherein the milvexian is administered orally as an aqueous dispersion by dispersing the immediate-release film-coated tablet in an aqueous medium in less than 60 seconds at 37° C.
Aspect 68. The method of any one of any one of aspects 53-67, wherein the immediate-release film-coated tablet is administered twice daily for at least 13 weeks.
Aspect 69. The method of any one of any one of aspects 53-68, wherein the immediate-release film-coated tablet is administered once in the morning and once in the evening.
Aspect 70. The method of aspect 69, wherein the morning administration is made at the same time each day and the evening administration is made at the same time each day.
Aspect 71. The method of any one of aspects 53-70, wherein the administration reduces FXI clotting activity in the patient by about 27% to about 64% relative to the baseline FXI clotting activity.
Aspect 72. The method of any one of any one of aspects 53-71, wherein the administration results in a prolongation of activated partial thromboplastin time (aPTT) in the patient with a ratio to baseline of 2.1 to 2.6.
Aspect 73. The method of any one of the preceding aspects, wherein the method further comprises administering a standard-of-care antiplatelet therapy to the patient.
Aspect 74. The method of aspect 73, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.
Aspect 75. The method of any one of aspects 73 and 74, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.
Aspect 76. The method of any one of aspects 73-75, wherein the standard-of-care antiplatelet therapy is aspirin, clopidogrel, ticagrelor, or prasugrel.
Aspect 77. The method of any one of aspects 73-76, wherein the standard-of-care antiplatelet therapy is clopidogrel, ticagrelor, or prasugrel.
Aspect 78. The method of any one of aspects 73-77, wherein the standard-of-care antiplatelet therapy comprises a single antiplatelet therapy (SAPT).
Aspect 79. The method of aspect 78, wherein the single antiplatelet therapy comprises aspirin, or a P2Y12 inhibitor selected from clopidogrel, ticagrelor, and prasugrel.
Aspect 80. The method of any one of aspects 73-77, wherein the standard-of-care antiplatelet therapy comprises a dual antiplatelet therapy (DAPT).
Aspect 81. The method of aspect 80, wherein the dual antiplatelet therapy comprises aspirin and a P2Y12 inhibitor selected from clopidogrel, ticagrelor, or prasugrel.
Aspect 82. The method of any one of aspects 80 and 81, wherein the dual antiplatelet therapy comprises a aspirin and a P2Y12 inhibitor is clopidogrel, ticagrelor, or prasugrel.
Aspect 83. The method of any one of aspects 80-82, wherein the dual antiplatelet therapy comprises aspirin and clopidogrel.
Aspect 84. The method of any one of aspects 80-83, wherein the dual antiplatelet therapy (DAPT) is administered for 21 days, followed by single antiplatelet therapy (SAPT) thereafter.
Aspect 85. The method of any one of aspects 80-83, wherein the dual antiplatelet therapy (i.e., aspirin and P2Y12 inhibitor) is administered for more than 90 days (with or without de-escalation to SAPT).
Aspect 86. Milvexian for use in preventing one or more of stroke and non-central nervous system (CNS) systemic embolism events in a human patient with atrial fibrillation, wherein the method comprises administering to the human patient a pharmaceutical composition comprising about 100 mg of milvexian (or a pharmaceutically acceptable salt or solvate thereof, on a milvexian basis); wherein the pharmaceutical composition is administered twice daily.
Aspect 87. Milvexian for use in preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising 100 mg of milvexian (or a pharmaceutically acceptable salt or solvate thereof, on a milvexian basis); wherein the composition is administered twice daily.
Aspect 88. Milvexian for use of aspect 87, wherein the adverse cardiovascular event is cardiovascular death.
Aspect 89. Milvexian for use of any one of aspects 86-88, wherein the patient has atrial fibrillation, and also has concomitant coronary artery disease (CAD) and/or peripheral artery disease (PAD).
Aspect 90. Milvexian for use of any one of aspects 86-89, wherein the pharmaceutical composition is a solid oral pharmaceutical composition comprising milvexian (or a pharmaceutically acceptable salt or solvate thereof) and one or more pharmaceutically acceptable excipients.
Aspect 91. Milvexian for use of aspect 90, wherein the solid oral pharmaceutical composition is an immediate release tablet.
Aspect 92. Milvexian for use of aspect 91, wherein the immediate release tablet has a disintegration time in water of less than 20 seconds at 37° C.
Aspect 93. Milvexian for use of any one of aspects 86-92, wherein the administration results in a milvexian plasma half-life ranging from about 13 hours to about 16 hours.
Aspect 94. Milvexian for use of any one of aspects 86-93, wherein the administration results in a milvexian plasma half-life ranging from about 13 hours to about 16 hours during repeat dosing.
Aspect 95. Milvexian for use of any one of aspects 86-94, wherein the administration results in milvexian plasma concentrations reaching steady-state at about 4 days to 6 days.
Aspect 96. Milvexian for use of any one of aspects 86-95, wherein the pharmaceutical composition comprising milvexian (or a pharmaceutically acceptable salt or solvate thereof) is administered without regard to the timing of food intake.
Aspect 97. Milvexian for use of any one of aspects 86-96, wherein the composition comprising milvexian (or a pharmaceutically acceptable salt or solvate thereof) is administered twice daily for at least 13 weeks.
Aspect 98. Milvexian for use of any one of aspects 86-97, wherein the composition comprising milvexian (or a pharmaceutically acceptable salt or solvate thereof) is administered once in the morning and once in the evening.
Aspect 99. Milvexian for use of any one of aspects 86-98, wherein the morning administration is made at the same time each day and the evening administration is made at the same time each day.
Aspect 100. Milvexian for use in preventing one or more of stroke and non-central nervous system (CNS) systemic embolism in a patient with atrial fibrillation, wherein the method comprising administering to the patient an immediate release film coated tablet comprising 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (vi) age between 65 and 74 years;
      (vii) hypertension;
      (viii) diabetes mellitus;
      (ix) history of coronary artery disease (CAD) (including post myocardial infarction or acute coronary syndrome event with or without percutaneous coronary intervention (PCI)) or peripheral artery disease (PAD); and
      (x) history of heart failure.
Aspect 101. Milvexian for use in preventing one or more adverse cardiovascular events in a patient with atrial fibrillation, wherein each event is selected from the group consisting of cardiovascular death, myocardial infarction, stroke, and non-central nervous system (CNS) systemic embolism said method comprising administering to the patient an immediate release film-coated tablet comprising 100 mg of milvexian, or a pharmaceutically acceptable salt thereof, and one or more pharmaceutically acceptable excipient,
   wherein the immediate release film-coated tablet is administered twice a day with or without a meal,
   wherein the patient is at least 18 years old;
   wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 102. Milvexian for use of aspect 101, wherein the adverse cardiovascular event is cardiovascular death.
Aspect 103. Milvexian for use of any one of aspects 100-102, wherein immediate release film-coated tablet has a disintegration time in water of less than 20 seconds at 37° C.
Aspect 104. Milvexian for use of any one of aspects 100-103, wherein the administered milvexian has a plasma half-life of 13-16 hours.
Aspect 105. Milvexian for use of any one of aspects 100-104, wherein the administered milvexian has a plasma half-life of 13-16 hours during repeat dosing.
Aspect 106. Milvexian for use of any one of aspects 100-105, wherein the composition comprising milvexian (or a pharmaceutically acceptable salt or solvate thereof) is administered twice daily for at least 13 weeks.
Aspect 107. Milvexian for use of any one of aspects 86-106, wherein the composition comprising milvexian (or a pharmaceutically acceptable salt or solvate thereof) is administered once daily in the morning and once daily in the evening.
Aspect 108. Milvexian for use of aspect 107, wherein the morning administration is made at the same time each day and the evening administration is made at the same time each day.
Aspect 109. Milvexian for use of any one of aspects 86-108, wherein the administration reduces FXI clotting activity in the patient by about 27% to about 64% relative to the baseline FXI clotting activity.
Aspect 110. Milvexian for use of any one of aspects 86-109, wherein the administration results in a prolongation of activated partial thromboplastin time (aPTT) in the patient with a ratio to baseline of 2.1 to 2.6.
Aspect 111. Milvexian for use in preventing stroke in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 112. Milvexian for use in preventing one or more non-central nervous system (CNS) systemic embolism in a human patient with atrial fibrillation, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 113. Milvexian for use in preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.
Aspect 114. Milvexian for use of any one of aspects 111-113, wherein the adverse cardiovascular event is cardiovascular death.
Aspect 115. Milvexian for use of any one of aspects 111-113, wherein the adverse cardiovascular event is myocardial infarction.
Aspect 116. Milvexian for use of any one of aspects 111-113, wherein the adverse cardiovascular event is stroke.
Aspect 117. Milvexian for use of any one of aspects 111-113, wherein the adverse cardiovascular event is non-central nervous system (CNS) systemic embolism.
Aspect 118. Milvexian for use of any one of aspects 111-117, wherein the patient has atrial fibrillation, and also has concomitant coronary artery disease (CAD) and/or peripheral artery disease (PAD).
Aspect 119. Milvexian for use of aspect 118, wherein the patient has atrial fibrillation, and also has concomitant coronary artery disease (CAD).
Aspect 120. Milvexian for use of aspect 118, wherein the patient has atrial fibrillation, and also has concomitant peripheral artery disease (PAD).
Aspect 121. Milvexian for use of any one of aspects 111-120, wherein the pharmaceutical composition is a solid oral pharmaceutical composition comprising milvexian (or a pharmaceutically acceptable salt or solvate thereof) and one or more pharmaceutically acceptable excipients.
Aspect 122. Milvexian for use of aspect 121, wherein the solid oral pharmaceutical composition is an immediate release tablet.
Aspect 123. Milvexian for use of aspect 122, wherein the immediate release tablet has a disintegration time in water of less than 20 seconds at 37° C.
Aspect 124. Milvexian for use of any one of aspects 111-123, wherein the administration results in a milvexian plasma half-life ranging from about 13 hours to about 16 hours.
Aspect 125. Milvexian for use of any one of aspects 111-124, wherein the administration results in a milvexian plasma half-life ranging from about 13 hours to about 16 hours during repeat dosing.
Aspect 126. Milvexian for use of any one of aspects 111-125, wherein the administration results in milvexian plasma concentrations reaching steady-state at about 4 days to 6 days.
Aspect 127. Milvexian for use of any one of aspects 111-126, wherein the pharmaceutical composition comprising milvexian (or a pharmaceutically acceptable salt or solvate thereof) is administered without regard to the timing of food intake.
Aspect 128. Milvexian for use of any one aspects 111-127, wherein the patient is unable to swallow a tablet dosage form.
Aspect 129. Milvexian for use of any one aspects 111-128, wherein the pharmaceutical composition is dispersed in an aqueous medium to form an aqueous dispersion.
Aspect 130. Milvexian for use of aspect 129, wherein the aqueous medium is water, saline, phosphate buffer, vegetable juice, or fruit juice, including, for example, apple sauce.
Aspect 131. Milvexian for use of aspect 129 or aspect 130, wherein the aqueous dispersion is administered to the human patient via a nasogastric tube or spoon.
Aspect 132. Milvexian for use of any one of aspects 129-131, wherein Milvexian is administered orally as an aqueous dispersion by dispersing the pharmaceutical composition in an aqueous medium in less than 60 seconds at 37° C.
Aspect 133. Milvexian for use of any one of any one of aspects 111-132, wherein the composition comprising milvexian (or a pharmaceutically acceptable salt or solvate thereof) is administered twice daily for at least 13 weeks.
Aspect 134. Milvexian for use of any one of any one of aspects 111-133, wherein the composition comprising milvexian (or a pharmaceutically acceptable salt or solvate thereof) is administered once daily in the morning and once daily in the evening.
Aspect 135. Milvexian for use of aspect 134, wherein the morning administration is made at the same time each day and the evening administration is made at the same time each day.
Aspect 136. Milvexian for use of any one of aspects 111-135, wherein the administration reduces FXI clotting activity in the patient by about 27% to about 64% relative to the baseline FXI clotting activity.
Aspect 137. Milvexian for use of any one of any one of aspects 111-136, wherein the administration results in a prolongation of activated partial thromboplastin time (aPTT) in the patient with a ratio to baseline of 2.1 to 2.6.
Aspect 138. Milvexian for use in preventing stroke in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 139. Milvexian for use in preventing non-central nervous system (CNS) systemic embolism events in a patient with atrial fibrillation, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 140. Milvexian for use of any one of aspects 138-139, wherein the patient has atrial fibrillation, and also has concomitant coronary artery disease (CAD) and/or peripheral artery disease (PAD).
Aspect 141. Milvexian for use of aspect 140, wherein the patient has atrial fibrillation, and also has concomitant coronary artery disease (CAD).
Aspect 142. Milvexian for use of aspect 140, wherein the patient has atrial fibrillation, and also has concomitant peripheral artery disease (PAD).
Aspect 143. Milvexian for use of any one of aspects 138-142, wherein the immediate-release film-coated tablet has a disintegration time in water of less than 20 seconds at 37° C.
Aspect 144. Milvexian for use of any one of aspects 138-143, wherein the administration results in a milvexian plasma half-life ranging from about 13 hours to about 16 hours.
Aspect 145. Milvexian for use of any one of aspects 138-144, wherein the administration results in a milvexian plasma half-life ranging from about 13 hours to about 16 hours during repeat dosing.
Aspect 146. Milvexian for use of any one of aspects 138-145, wherein the administration results in milvexian plasma concentrations reaching steady-state at about 4 days to 6 days.
Aspect 147. Milvexian for use of any one of aspects 138-146, wherein the immediate-release film-coated tablet is administered without regard to the timing of food intake.
Aspect 148. Milvexian for use of any one aspects 138-147, wherein the patient is unable to swallow a tablet dosage form.
Aspect 149. Milvexian for use of any one aspects 138-148, wherein the immediate-release film-coated tablet is dispersed in an aqueous medium to form an aqueous dispersion.
Aspect 150. Milvexian for use of aspect 149, wherein the aqueous medium is water, saline, phosphate buffer, vegetable juice, or fruit juice, including, for example, apple sauce.
Aspect 151. Milvexian for use of aspect 149 or aspect 150, wherein the aqueous dispersion is administered to the human patient via a nasogastric tube or spoon.
Aspect 152. Milvexian for use of any one of aspects 138-151, wherein the milvexian is administered orally as an aqueous dispersion by dispersing the immediate-release film-coated tablet in an aqueous medium in less than 60 seconds at 37° C.
Aspect 153. Milvexian for use of any one of any one of aspects 138-152, wherein the immediate-release film-coated tablet is administered twice daily for at least 13 weeks.
Aspect 154. Milvexian for use of any one of any one of aspects 138-153, wherein the immediate-release film-coated tablet is administered once daily in the morning and once daily in the evening.
Aspect 155. Milvexian for use of aspect 154, wherein the morning administration is made at the same time each day and the evening administration is made at the same time each day.
Aspect 156. Milvexian for use of any one of aspects 138-155, wherein the administration reduces FXI clotting activity in the patient by about 27% to about 64% relative to the baseline FXI clotting activity.
Aspect 157. Milvexian for use of any one of any one of aspects 138-156, wherein the administration results in a prolongation of activated partial thromboplastin time (aPTT) in the patient with a ratio to baseline of 2.1 to 2.6.
Aspect 158. Milvexian for use of any one of aspects 86-157, wherein the use further comprises administering a standard-of-care antiplatelet therapy to the patient.
Aspect 159. Milvexian for use of aspect 158, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.
Aspect 160. Milvexian for use of aspects 158 and 159, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.
Aspect 161. Milvexian for use of any one of aspects 158-160, wherein the standard-of-care antiplatelet therapy is selected from aspirin, clopidogrel, ticagrelor, or prasugrel.
Aspect 162. Milvexian for use of any one of aspects 158-161, wherein the standard-of-care antiplatelet therapy is selected from clopidogrel, ticagrelor, or prasugrel.
Aspect 163. Milvexian for use of any one of aspects 158-162, wherein the standard-of-care antiplatelet therapy comprises a single antiplatelet therapy (SAPT).
Aspect 164. Milvexian for use of aspect 163, wherein the single antiplatelet therapy comprises aspirin, or a P2Y12 inhibitor selected from clopidogrel, ticagrelor, or prasugrel.
Aspect 165. Milvexian for use of any one of aspects 158-164, wherein the standard-of-care antiplatelet therapy comprises a dual antiplatelet therapy (DAPT).
Aspect 166. Milvexian for use of aspect 165, wherein the dual antiplatelet therapy comprises aspirin and a P2Y12 inhibitor selected from clopidogrel, ticagrelor, or prasugrel.
Aspect 167. Milvexian for use of any one of aspects 165 and 166, wherein the dual antiplatelet therapy comprises aspirin and a P2Y12 inhibitor selected from clopidogrel, ticagrelor, or prasugrel.
Aspect 168. Milvexian for use of any one of aspects 165-167, wherein the dual antiplatelet therapy comprises a aspirin and clopidogrel.
Aspect 169. Milvexian for use of any one of aspects 165-168, wherein the dual antiplatelet therapy (DAPT) is administered for 21 days, followed by single antiplatelet therapy (SAPT) thereafter.
Aspect 170. Milvexian for use of any one of aspects 165-168, wherein the dual antiplatelet therapy (i.e., aspirin and P2Y12 inhibitor) is administered for more than 90 days (with or without de-escalation to SAPT).
Aspect 171. A method for preventing one or more of stroke and non-central nervous system (CNS) systemic embolism in a human patient with atrial fibrillation, wherein the method comprises administering to the human patient a pharmaceutical composition comprising about 100 mg of milvexian, or a pharmaceutically acceptable salt thereof; wherein the pharmaceutical composition is administered twice daily.
Aspect 172. A method for preventing one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.
Aspect 173. A method for reducing the risk of one or more of stroke and non-central nervous system (CNS) systemic embolism in a human patient with atrial fibrillation, wherein the method comprises administering to the human patient a pharmaceutical composition comprising about 100 mg of milvexian, or a pharmaceutically acceptable salt thereof; wherein the pharmaceutical composition is administered twice daily.
Aspect 174. A method for reducing the risk of one or more of adverse cardiovascular events in a patient with atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.
Aspect 175. A method for preventing one or more of stroke and non-central nervous system (CNS) systemic embolism events in a human patient with a history of atrial fibrillation, wherein the method comprises administering to the human patient a pharmaceutical composition comprising 100 mg of milvexian (or a pharmaceutically acceptable salt or solvate thereof, on a milvexian basis); wherein the pharmaceutical composition is administered twice daily.
Aspect 176. A method for preventing one or more of adverse cardiovascular events in a patient with a history of atrial fibrillation, wherein each event is cardiovascular death, myocardial infarction, stroke, or non-central nervous system (CNS) systemic embolism, wherein the method comprises administering to the human patient a pharmaceutical composition comprising 100 mg of milvexian (or a pharmaceutically acceptable salt or solvate thereof, on a milvexian basis); wherein the composition is administered twice daily.
Aspect 177. The method of aspect 176, wherein the adverse cardiovascular event is cardiovascular death.
Aspect 178. The method of any one of aspects 171-177, wherein the patient has a history of atrial fibrillation, and also has concomitant coronary artery disease (CAD) and/or peripheral artery disease (PAD).
Aspect 179. The method of any one of aspects 171-178, wherein the pharmaceutical composition comprises 100 mg of milvexian (or a pharmaceutically acceptable salt or solvate thereof, on a milvexian basis).
Aspect 180. The method of any one of aspects 171-179, wherein the pharmaceutical composition is a solid oral pharmaceutical composition comprising milvexian (or a pharmaceutically acceptable salt or solvate thereof) and one or more pharmaceutically acceptable excipients.
Aspect 181. The method of aspect 180, wherein the solid oral pharmaceutical composition is an immediate release tablet.
Aspect 182. The method of aspect 181, wherein the immediate release tablet has a disintegration time in water of less than 20 seconds at 37° C.
Aspect 183. The method of any one of aspects 180-182, wherein the administration results in a milvexian plasma half-life ranging from about 13 hours to about 16 hours.
Aspect 184. The method of any one of aspects 171-183, wherein the administration results in milvexian plasma concentrations reaching steady-state at about 4 days to 6 days.
Aspect 185. The method of any one of aspects 171-184, wherein the pharmaceutical composition comprising milvexian (or a pharmaceutically acceptable salt or solvate thereof) is administered without regard to the timing of food intake.
Aspect 186. A method of preventing one or more of stroke and non-central nervous system (CNS) systemic embolism events in a patient with a history of atrial fibrillation, wherein the method comprises administering to the patient an immediate release film coated tablet comprising 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.
Aspect 187. A method of preventing one or more adverse cardiovascular events in a patient with a history of atrial fibrillation, wherein each event is selected from the group consisting of cardiovascular death, myocardial infarction, stroke, and non-central nervous system (CNS) systemic embolism said method comprising administering to the patient an immediate release film-coated tablet comprising 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.
Aspect 188. The method of aspect 187, wherein the adverse cardiovascular event is cardiovascular death.
Aspect 189. The method of any one of aspects 186-188, wherein immediate release film-coated tablet has a disintegration time in water of less than 20 seconds at 37° C.
Aspect 190. The method of any one of aspects 186-189, wherein the administered milvexian has a plasma half-life of 13-16 hours.
Aspect 191. The method of any one of aspects 171-190, wherein the administration reduces FXI clotting activity in the patient by about 27% to about 64% relative to the baseline FXI clotting activity.
Aspect 192. The method of any one of aspects 171-191, wherein the administration results in a prolongation of activated partial thromboplastin time (aPTT) in the patient with a ratio to baseline of 2.1 to 2.6.
Aspect 193. The method of any one of aspects 171-192, wherein the composition comprising milvexian (or a pharmaceutically acceptable salt or solvate thereof) is administered twice daily for at least 13 weeks.
Aspect 194. The method of any one of aspects 171-193, wherein the composition comprising milvexian (or a pharmaceutically acceptable salt or solvate thereof) is administered once daily in the morning and once daily in the evening.
Aspect 195. The method of aspect 194, wherein the morning administration is made at the same time each day and the evening administration is made at the same time each day.
Aspect 196. Milvexian for use in a method of preventing one or more of stroke and non-central nervous system (CNS) systemic embolism events in a human patient with a history of atrial fibrillation, wherein the method comprises administering to the human patient a pharmaceutical composition comprising 100 mg, of milvexian (or a pharmaceutically acceptable salt or solvate thereof, on a milvexian basis); wherein the pharmaceutical composition is administered twice daily.
Aspect 197. Milvexian for use in a method for preventing one or more of adverse cardiovascular events in a patient with a history of atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising 100 mg, of milvexian (or a pharmaceutically acceptable salt or solvate thereof, on a milvexian basis); wherein the composition is administered twice daily.
Aspect 198. Milvexian for use of aspect 197, wherein the adverse cardiovascular event is cardiovascular death.
Aspect 199. Milvexian for use of any one of aspects 196-198, wherein the patient has a history of atrial fibrillation, and also has concomitant coronary artery disease (CAD) and/or peripheral artery disease (PAD).
Aspect 200. Milvexian for use of any one of aspects 196-199, wherein the pharmaceutical composition comprises 100 mg of milvexian (or a pharmaceutically acceptable salt or solvate thereof, on a milvexian basis).
Aspect 201. Milvexian for use of any one of aspects 196-200, wherein the pharmaceutical composition is a solid oral pharmaceutical composition comprising milvexian (or a pharmaceutically acceptable salt or solvate thereof) and one or more pharmaceutically acceptable excipients.
Aspect 202. Milvexian for use of aspect 201, wherein the solid oral pharmaceutical composition is an immediate release tablet.
Aspect 203. Milvexian for use of aspect 202, wherein the immediate release tablet has a disintegration time in water of less than 20 seconds at 37° C.
Aspect 204. Milvexian for use of any one of aspects 201-203, wherein the administration results in a milvexian plasma half-life ranging from about 13 hours to about 16 hours.
Aspect 205. Milvexian for use of any one of aspects 196-204, wherein the administration results in milvexian plasma concentrations reaching steady-state at about 6 days.
Aspect 206. Milvexian for use of any one of aspects 196-205, wherein the pharmaceutical composition comprising milvexian (or a pharmaceutically acceptable salt or solvate thereof) is administered without regard to the timing of food intake.
Aspect 207. Mivexian for use in a method of preventing one or more of stroke and non-central nervous system (CNS) systemic embolism events in a patient with a history of atrial fibrillation, wherein the method comprising administering to the patient an immediate release film coated tablet comprising 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of Category (A) risk factors selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.
Aspect 208. Milvexian for use in a method of preventing one or more adverse cardiovascular events in a patient with a history of atrial fibrillation, wherein each event is cardiovascular death, myocardial infarction, stroke, or non-central nervous system (CNS) systemic embolism, said method comprising administering to the patient an immediate release film-coated tablet comprising 100 mg of milvexian, or a pharmaceutically acceptable salt thereof, and one or more pharmaceutically acceptable excipient,
wherein the immediate release film-coated tablet is administered twice a day with or without a meal,
wherein the patient is at least 18 years old;
wherein the patient further has one or more of Category (A) risk factor selected from (i) age greater than or equal to 75 years or (ii) history of ischemic stroke or silent brain infarct; or two or more of concomitant or comorbid Category (B) cardiovascular risk factor selected from the group consisting of:
   (i) age between 65 and 74 years;
   (ii) hypertension;
   (iii) diabetes mellitus;
   (iv) history of coronary artery disease (CAD) (including post myocardial infarction or acute coronary syndrome event with or without percutaneous coronary intervention (PCI)) or peripheral artery disease (PAD); and
   (v) history of heart failure.
Aspect 209. Milvexian for use of aspect 208, wherein the adverse cardiovascular event is cardiovascular death.
Aspect 210. Milvexian for use of any one of aspects 207-209, wherein immediate release film-coated tablet has a disintegration time in water of less than 20 seconds.
Aspect 211. Milvexian for use of any one of aspects 207-210, wherein the administered milvexian has a plasma half-life of 13-16 hours.
Aspect 212. Milvexian for use of any one of aspects 196-211, wherein the administration reduces FXI clotting activity in the patient by about 27% to about 64% relative to the baseline FXI clotting activity.
Aspect 213. Milvexian for use of any one of aspects 196-212, wherein the administration results in a prolongation of activated partial thromboplastin time (aPTT) in the patient with a ratio to baseline of 2.1 to 2.6.
Aspect 214. Milvexian for use of any one of aspects 196-213, wherein the composition comprising milvexian (or a pharmaceutically acceptable salt or solvate thereof) is administered twice daily for at least 13 weeks.
Aspect 215. Milvexian for use of any one of aspects 196-214, wherein the composition comprising milvexian (or a pharmaceutically acceptable salt or solvate thereof) is administered once in the morning and once in the evening.
Aspect 216. Milvexian for use of aspect 215, wherein the morning administration is made at the same time each day and the evening administration is made at the same time each day.
Aspect 217. The method of any one of the preceding aspects, wherein the milvexian administration results in no clinically significant QTc interval prolongation.
Aspect 218. Milvexian for use of any one of the preceding aspects, wherein the milvexian administration results in no clinically significant QTc interval prolongation.
Aspect 219. Milvexian for use of any one of the preceding aspects, wherein the milvexian administration results in no clinically meaningful change to prothrombin time, with a maximal mean percent change from baseline of approximately 5%.
Aspect 220. Milvexian for use of any one of the preceding aspects, wherein the administration results in no clinically relevant effects on any bleeding caused by milvexian exposure as measured by steady state AUC or Cₘₐₓ.
Aspect 221. Milvexian for use of any one of the preceding aspects, wherein the administration results in minimal impairment of hemostasis in the human patient.
Aspect 222. The method of any one of the preceding aspects, wherein the milvexian administration results in no clinically meaningful change to prothrombin time, with a maximal mean percent change from baseline of approximately 5%.
Aspect 223. The method of any one of the preceding aspects, wherein the administration results in no clinically relevant effects on any bleeding caused by milvexian exposure as measured by steady state AUC or Cₘₐₓ.

Aspect 224. The method of any one of the preceding aspects, wherein the administration results in minimal impairment of hemostasis in the human patient.
Aspect 225. The method of any one of aspects 171-195, wherein the regimen comprises an immediate release tablet.
Aspect 226. The method of aspect 225, wherein the immediate release tablet has a disintegration time in water of less than 20 seconds at 37° C.
Aspect 227. The method of any one of aspects 171-195, wherein the administration results in a milvexian plasma terminal half-life ranging from about 13 hours to about 16 hours.
Aspect 228. The method of any one of aspects 171-195, wherein the regimen is administered without regard to the timing of food intake.
Aspect 229. The method of any one of aspects 171-195, wherein the immediate release tablet is dispersed in an aqueous medium to form an aqueous dispersion.
Aspect 230. The method of aspect 229, wherein the aqueous medium comprises water or apple sauce.
Aspect 231. A method of preventing stroke or non-central nervous system (CNS) systemic embolism in a human patient with atrial fibrillation, wherein the method comprises orally administering to the human patient a regimen comprising about 100 mg of milvexian twice daily; wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 1194 ng/ml to about 2326 ng/ml, (ii) a steady state Cₘₐₓ mean (std) of 1760 (566) ng/mL, (iii) a steady state AUC₀₋₂₄ ranging from about 23300 ng*h/mL to about 49100 ng*h/mL, or (iv) a steady state AUC₀₋₂₄ mean (std) of 36200 (12900) ng*h/mL; (3) no clinically significant QTc interval prolongation, (4) no clinically relevant effects on any bleeding caused by milvexian exposure as measured by steady state AUC or Cₘₐₓ; (5) minimal impairment of hemostasis in the human patient, (6) no clinically meaningful change to prothrombin time, with a maximal mean percent change from baseline of approximately 5%; or (7) any combination of the aforementioned clinical outcomes.
Aspect 232. A method for preventing one or more of stroke, or non-central nervous system (CNS) systemic embolism in a patient with atrial fibrillation, wherein the method comprises orally administering to the human patient a regimen comprising about 100 mg of milvexian twice daily.
Aspect 233. A method for preventing one or more of cardiovascular death, myocardial infarction, stroke, or non-central nervous system (CNS) systemic embolism in a patient with atrial fibrillation, wherein the method comprises orally administering to the human patient a regimen comprising about 100 mg of milvexian twice daily.
Aspect 234. A method for preventing stroke in a human patient with atrial fibrillation, wherein the method comprises orally administering to the human patient a regimen comprising about 100 mg of milvexian twice daily.
Aspect 235. A method for preventing ischemic stroke in a human patient with atrial fibrillation, wherein the method comprises orally administering to the human patient a regimen comprising about 100 mg of milvexian twice daily.
Aspect 236. A method for preventing non-central nervous system (CNS) systemic embolism in a human patient with atrial fibrillation, wherein the method comprises orally administering to the human patient a regimen comprising 100 mg of milvexian twice daily.
Aspect 237. A method for preventing one or more of all cause death, myocardial infarction, stroke, and non-central nervous system (CNS) systemic embolism in a patient with atrial fibrillation, wherein the method comprises orally administering to the human patient a regimen comprising about 100 mg of milvexian twice daily.
Aspect 238. A method for preventing cardiovascular death events in a human patient with atrial fibrillation, wherein the method comprises orally administering to the human patient a regimen comprising about 100 mg of milvexian twice daily.
Aspect 239. A method for preventing all cause death in a human patient with atrial fibrillation, wherein the method comprises orally administering to the human patient a regimen comprising about 100 mg of milvexian twice daily.
Aspect 240. A method for preventing cardiovascular death, myocardial infarction, stroke, acute limb ischemia, deep vein thrombosis, or pulmonary embolism events in a human patient with atrial fibrillation, wherein the method comprises orally administering to the human patient a regimen comprising about 100 mg of milvexian twice daily.
Aspect 241. A method for preventing myocardial infarction in a human patient with atrial fibrillation, wherein the method comprises orally administering to the human patient a regimen comprising about 100 mg of milvexian twice daily.
Aspect 242. A method for preventing acute limb ischemia in a human patient with atrial fibrillation, wherein the method comprises orally administering to the human patient a regimen comprising about 100 mg of milvexian twice daily.
Aspect 243. A method for preventing deep vein thrombosis in a human patient with atrial fibrillation, wherein the method comprises orally administering to the human patient a regimen comprising about 100 mg of milvexian twice daily.
Aspect 244. A method for preventing pulmonary embolism in a human patient with atrial fibrillation, wherein the method comprises orally administering to the human patient a regimen comprising 100 mg milvexian twice daily.
Aspect 245. The method of any one of aspects 231-244, wherein the administration results in a steady state plasma concentration profile of milvexian in about 3 to 6 days.
Aspect 246. The method of any one of aspects 231-245, wherein the administration results in a steady state plasma concentration profile of milvexian in about 6 days.
Aspect 247. The method of any one of aspects 231-246, wherein the administration results in a steady state plasma concentration profile of milvexian characterized by:
   (i) a Cₘₐₓ at steady state ranging from about 1194 ng/mL to about 2326 ng/ml,
   (ii) a steady state Cₘₐₓ mean (std) of 1760 (566) ng/ml;
   (iii) a steady state AUC₀₋₂₄ ranging from about 23300 ng*h/mL to about 49100 ng*h/mL; or
   (iv) a steady state AUC₀₋₂₄ mean (std) of 36200 (12900) ng*h/mL.
Aspect 248. The method of any one of aspects 231-247, wherein the administration results in no clinically significant QTc interval prolongation.
Aspect 249. The method of any one of aspects 231-248, wherein the administration results in no clinically relevant effects on any bleeding caused by milvexian exposure as measured by steady state AUC or Cₘₐₓ.
Aspect 250. The method of any one of aspects 231-249, wherein the administration results in minimal impairment of hemostasis in the human patient.
Aspect 251. The method of any one of aspects 231-249, wherein the administration results in no clinically meaningful change to prothrombin time, with a maximal mean percent change from baseline of approximately 5%.
Aspect 252. The method of any one of aspects 231-251, wherein the regimen comprises an immediate release tablet.
Aspect 253. The method of aspect 252, wherein the immediate release tablet has a disintegration time in water of less than 20 seconds at 37° C.
Aspect 254. The method of any one of aspects 231-253, wherein the administration results in a milvexian plasma terminal half-life ranging from about 13 hours to about 16 hours.
Aspect 255. The method of any one of aspects 231-254, wherein the regimen is administered without regard to the timing of food intake.
Aspect 256. The method of any one of aspects 252-255, wherein the immediate release tablet is dispersed in an aqueous medium to form an aqueous dispersion.
Aspect 257. The method of aspect 256, wherein the aqueous medium comprises water or apple sauce.
Aspect 258. Milvexian for use in a method of preventing stroke or non-central nervous system (CNS) systemic embolism in a human patient with atrial fibrillation, wherein the method comprises orally administering to the human patient a regimen comprising about 100 mg of milvexian twice daily; wherein the method achieves at least one of the following clinical outcomes: (1) reaches a steady state plasma concentration profile of milvexian in about 6 days; (2) reaches a steady state plasma concentration profile of milvexian characterized by: (i) a steady state Cₘₐₓ ranging from about 1194 ng/ml to about 2326 ng/ml, (ii) a steady state Cₘₐₓ mean (std) of 1760 (566) ng/mL, (iii) a steady state AUC₀₋₂₄ ranging from about 23300 ng*h/mL to about 49100 ng*h/mL, or (iv) a steady state AUC₀₋₂₄ mean (std) of 36200 (12900) ng*h/mL; (3) a change from baseline in QTc in the patient of less than 10 millisecond, (4) no clinically relevant effects on any bleeding caused by milvexian exposure as measured by steady state AUC or Cmax; (5) minimal impairment of hemostasis in the human patient, (6) no clinically meaningful change to prothrombin time, with a maximal mean percent change from baseline of approximately 5%; or (7) any combination of the aforementioned outcomes.
Aspect 259. Milvexian for use in a method for preventing one or more of stroke, or non-central nervous system (CNS) systemic embolism in a patient with a history of atrial fibrillation, wherein the method comprises orally administering to the human patient a regimen comprising about 100 mg of milvexian twice daily.
Aspect 260. Milvexian for use in a method for preventing one or more of cardiovascular death, myocardial infarction, stroke, or non-central nervous system (CNS) systemic embolism in a patient with atrial fibrillation, wherein the method comprises orally administering to the human patient a regimen comprising about 100 mg of milvexian twice daily.
Aspect 261. Milvexian for use in a method for preventing stroke in a human patient with a history of atrial fibrillation, wherein the method comprises orally administering to the human patient a regimen comprising about 100 mg of milvexian twice daily.
Aspect 262. Milvexian for use in a method for preventing ischemic stroke in a human patient with a history of atrial fibrillation, wherein the method comprises orally administering to the human patient a regimen comprising about 100 mg of milvexian twice daily.
Aspect 263. Milvexian for use in a method for preventing non-central nervous system (CNS) systemic embolism in a human patient with a history of atrial fibrillation, wherein the method comprises orally administering to the human patient a regimen comprising about 100 mg of milvexian twice daily.
Aspect 264. Milvexian for use in a method for preventing one or more of all cause death, myocardial infarction, stroke, or non-central nervous system (CNS) systemic embolism in a patient with atrial fibrillation, wherein the method comprises orally administering to the human patient a regimen comprising about 100 mg of milvexian twice daily.
Aspect 265. Milvexian for use in a method for preventing cardiovascular death in a human patient with atrial fibrillation, wherein the method comprises orally administering to the human patient a regimen comprising about 100 mg of milvexian twice daily.
Aspect 266. Milvexian for use in a method for preventing all cause death in a human patient with atrial fibrillation, wherein the method comprises orally administering to the human patient a regimen comprising about 100 mg of milvexian twice daily.
Aspect 267. Milvexian for use in a method for preventing cardiovascular death, myocardial infarction, stroke, acute limb ischemia, deep vein thrombosis, or pulmonary embolism in a human patient with a history of atrial fibrillation, wherein the method comprises orally administering to the human patient a regimen comprising about 100 mg of milvexian twice daily.
Aspect 268. Milvexian for use in a method for preventing myocardial infarction in a human patient with a history of atrial fibrillation, wherein the method comprises orally administering to the human patient a regimen comprising about 100 mg of milvexian twice daily.
Aspect 269. Milvexian for use in a method for preventing acute limb ischemia in a human patient with a history of atrial fibrillation, wherein the method comprises orally administering to the human patient a regimen comprising about 100 mg of milvexian twice daily.
Aspect 270. Milvexian for use in a method for preventing deep vein thrombosis in a human patient with a history of atrial fibrillation, wherein the method comprises orally administering to the human patient a regimen comprising about 100 mg of milvexian twice daily.
Aspect 271. Milvexian for use in a method for preventing pulmonary embolism in a human patient with a history of atrial fibrillation, wherein the method comprises orally administering to the human patient a regimen comprising about 100 mg of milvexian twice daily.
Aspect 272. Milvexian for use of any one of aspects 258-271, wherein the administration results in a steady state plasma concentration profile of milvexian in about 4 to 6 days.
Aspect 273. Milvexian for use of any one of aspects 258-272, wherein the administration results in a steady state plasma concentration profile of milvexian in about 6 days.
Aspect 274. Mmilvexian for use of any one of aspects 258-273, wherein the administration results in a steady state plasma concentration profile of milvexian characterized by:
   (i) a Cₘₐₓ at steady state ranging from about 1194 ng/mL to about 2326 ng/ml,
   (ii) a steady state Cₘₐₓ mean (std) of 1760 (566) ng/mL;
   (iii) a steady state AUC₀₋₂₄ ranging from about 23300 ng*h/mL to about 49100 ng*h/mL; or
   (iv) a steady state AUC₀₋₂₄ mean (std) of 36200 (12900) ng*h/mL.
Aspect 275. Milvexian for use of any one of aspects 258-274, wherein the administration results in no clinically significant QTc interval prolongation.
Aspect 276. Milvexian for use of any one of aspects 258-275, wherein the administration results in no clinically relevant effects on any bleeding caused by milvexian exposure as measured by steady state AUC or Cₘₐₓ.
Aspect 277. Milvexian for use of any one of aspects 258-275, wherein the administration results in minimal impairment of hemostasis in the human patient.
Aspect 278. Milvexian for use of any one of aspects 258-275, wherein the administration results in no clinically meaningful change to prothrombin time, with a maximal mean percent change from baseline of approximately 5%.
Aspect 279. Milvexian for use of any one of aspects 258-278, wherein the regimen comprises an immediate release tablet.
Aspect 280. Milvexian for use of aspect 279, wherein the immediate release tablet has a disintegration time in water of less than 20 seconds at 37° C.
Aspect 281. Milvexian for use of any one of aspects 258-275, wherein the administration results in a milvexian plasma terminal half-life ranging from about 13 hours to about 16 hours.
Aspect 282. Milvexian for use of any one of aspects 258-275, wherein the regimen is administered without regard to the timing of food intake.
Aspect 283. Milvexian for use of any one of aspects 279-282, wherein the immediate release tablet is dispersed in an aqueous medium to form an aqueous dispersion.
Aspect 284. Milvexian for use of aspect 283, wherein the aqueous medium comprises water or apple sauce.
Aspect 285. Any one of aspects 1-284, wherein “100 mg of milvexian” is replaced by “50 mg of milvexian.”
Aspect 286. Any one of aspects 1-284, wherein “100 mg of milvexian” is replaced by “75 mg of milvexian.”
Aspect 287. Any one of aspects 1-284, wherein “100 mg of milvexian” is replaced by “87.5 mg of milvexian.”

### Aspect Set #2

Aspect 1. A method for preventing one or more of stroke and non-central nervous system (CNS) systemic embolism events in a human patient with a history of atrial fibrillation or flutter, wherein the method comprises administering to the human patient a pharmaceutical composition comprising about 50 mg, about 75.0 mg, about 87.5 mg, or about 100 mg of milvexian (or a pharmaceutically acceptable salt or solvate thereof, on a milvexian basis); wherein the pharmaceutical composition is administered twice daily.
Aspect 2. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg, about 75.0 mg, about 87.5 mg, or about 100 mg of milvexian (or a pharmaceutically acceptable salt or solvate thereof, on a milvexian basis); wherein the composition is administered twice daily.
Aspect 3. The method of aspect 2, wherein the adverse cardiovascular event is cardiovascular death.
Aspect 4. The method of any one of aspects 1-3, wherein the patient has a history of atrial fibrillation or flutter, and also has concomitant coronary artery disease (CAD) and/or peripheral artery disease (PAD).
Aspect 5. The method of any one of aspects 1-4, wherein the pharmaceutical composition comprises about 100 mg of milvexian (or a pharmaceutically acceptable salt or solvate thereof, on a milvexian basis).
Aspect 6. The method of any one of aspects 1-4, wherein the pharmaceutical composition comprises about 87.5 mg of milvexian (or a pharmaceutically acceptable salt or solvate thereof, on a milvexian basis).
Aspect 7. The method of any one of aspects 1-4, wherein the pharmaceutical composition comprises about 75 mg of milvexian (or a pharmaceutically acceptable salt or solvate thereof, on a milvexian basis).
Aspect 8. The method of any one of aspects 1-4, wherein the pharmaceutical composition comprises about 50 mg of milvexian (or a pharmaceutically acceptable salt or solvate thereof, on a milvexian basis).
Aspect 9. The method of any one of aspects 1-8, wherein the pharmaceutical composition is a solid oral pharmaceutical composition comprising milvexian (or a pharmaceutically acceptable salt or solvate thereof) and one or more pharmaceutically acceptable excipients.
Aspect 10. The method of aspect 9, wherein the solid oral pharmaceutical composition is an immediate release tablet.
Aspect 11. The method of aspect 10, wherein the immediate release tablet has a disintegration time in water of less than 20 seconds at 37° C.
Aspect 12. The method of any one of aspects 5-11, wherein the administration results in a milvexian plasma half-life ranging from about 13 hours to about 16 hours.
Aspect 13. The method of any one of aspects 5-11, wherein the administration results in a milvexian plasma half-life ranging from about 13 hours to about 16 hours during repeat dosing.
Aspect 14. The method of any one of the preceding aspects, wherein the administration results in milvexian plasma concentrations reaching steady-state at about 3 days to 6 days.
Aspect 15. The method of any one of the preceding aspects, wherein the pharmaceutical composition comprising milvexian (or a pharmaceutically acceptable salt or solvate thereof) is administered without regard to the timing of food intake.
Aspect 16. The method of any one of the preceding aspects, wherein the composition comprising milvexian (or a pharmaceutically acceptable salt or solvate thereof) is administered twice daily for at least 13 weeks.
Aspect 17. The method of any one of the preceding aspects, wherein the composition comprising milvexian (or a pharmaceutically acceptable salt or solvate thereof) is administered once daily in the morning and once daily in the evening.
Aspect 18. The method of any one of the preceding aspects, wherein the morning administration is made at the same time each day and the evening administration is made at the same time each day.
Aspect 19. A method of preventing one or more of stroke and non-central nervous system (CNS) systemic embolism events in a patient with a history of atrial fibrillation or flutter, wherein the method comprising administering to the patient an immediate release film coated tablet comprising about 50 mg, about 75.0 mg, about 87.5 mg, or about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (xi) age between 65 and 74 years;
      (xii) hypertension;
      (xiii) diabetes mellitus;
      (xiv) history of coronary artery disease (CAD) (including post myocardial infarction or acute coronary syndrome event with or without percutaneous coronary intervention (PCI)) or peripheral artery disease (PAD); and
      (xv) history of heart failure.
Aspect 20. A method of preventing one or more adverse cardiovascular events in a patient with a history of atrial fibrillation or flutter, wherein each event is selected from the group consisting of cardiovascular death, myocardial infarction, stroke, and non-central nervous system (CNS) systemic embolism said method comprising administering to the patient an immediate release film-coated tablet comprising about 50 mg, about 75.0 mg, about 87.5 mg, or 100 mg of milvexian, or a pharmaceutically acceptable salt thereof, and one or more pharmaceutically acceptable excipient,
   wherein the immediate release film-coated tablet is administered twice a day with or without a meal,
   wherein the patient is at least 18 years old;
   wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 21. The method of aspect 20, wherein the adverse cardiovascular event is stroke and non-central nervous system (CNS) systemic embolism.
Aspect 22. The method of any one of aspects 19-21, wherein immediate release film-coated tablet has a disintegration time in water of less than 20 seconds at 37° C.
Aspect 23. The method of any one of aspects 19-22, wherein the administered milvexian has a plasma half-life of 13-16 hours.
Aspect 24. The method of any one of aspects 19-23, wherein the administered milvexian has a plasma half-life of 13-16 hours during repeat dosing.
Aspect 25. The method of any one of aspects 19-24, wherein the composition comprising milvexian (or a pharmaceutically acceptable salt or solvate thereof) is administered twice daily for at least 13 weeks.
Aspect 26. The method of any one of aspects 19-25, wherein the composition comprising milvexian (or a pharmaceutically acceptable salt or solvate thereof) is administered once daily in the morning and once daily in the evening.
Aspect 27. The method of aspect 26, wherein the morning administration is made at the same time each day and the evening administration is made at the same time each day.
Aspect 28. The method of any one of the preceding aspects, wherein the administration reduces FXI clotting activity in the patient by about 27% to about 64% relative to the baseline FXI clotting activity.
Aspect 29. The method of any one of the preceding aspects, wherein the administration results in a prolongation of activated partial thromboplastin time (aPTT) in the patient with a ratio to baseline of 2.1 to 2.6.
Aspect 30. A method for preventing stroke in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.
Aspect 31. A method for preventing stroke in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.
Aspect 32. A method for preventing stroke in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.
Aspect 33. A method for preventing stroke in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 34. A method for preventing one or more non-central nervous system (CNS) systemic embolism events in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.
Aspect 35. A method for preventing one or more non-central nervous system (CNS) systemic embolism events in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.
Aspect 36. A method for preventing one or more non-central nervous system (CNS) systemic embolism events in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.
Aspect 37. A method for preventing one or more non-central nervous system (CNS) systemic embolism events in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 38. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising 50 mg of milvexian (or a pharmaceutically acceptable salt or solvate thereof, on a milvexian basis), wherein the composition is administered twice daily.
Aspect 39. The method of aspect 38, wherein the adverse cardiovascular event is cardiovascular death.
Aspect 40. The method of aspect 38, wherein the adverse cardiovascular event is myocardial infarction.
Aspect 41. The method of aspect 38, wherein the adverse cardiovascular event is stroke.
Aspect 42. The method of aspect 38, wherein the adverse cardiovascular event is non-central nervous system (CNS) systemic embolism.
Aspect 43. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.
Aspect 44. The method of aspect 43, wherein the adverse cardiovascular event is cardiovascular death.
Aspect 45. The method of aspect 43, wherein the adverse cardiovascular event is myocardial infarction.
Aspect 46. The method of aspect 43, wherein the adverse cardiovascular event is stroke.
Aspect 47. The method of aspect 43, wherein the adverse cardiovascular event is non-central nervous system (CNS) systemic embolism.
Aspect 48. The method of any one of aspects 30-47, wherein the patient has a history of atrial fibrillation or flutter, and also has concomitant coronary artery disease (CAD) and/or peripheral artery disease (PAD).
Aspect 49. The method of aspect 48, wherein the patient has a history of atrial fibrillation or flutter, and also has concomitant coronary artery disease (CAD).
Aspect 50. The method of aspect 48, wherein the patient has a history of atrial fibrillation or flutter, and also has concomitant peripheral artery disease (PAD).
Aspect 51. The method of any one of aspects 30-50, wherein the pharmaceutical composition is a solid oral pharmaceutical composition comprising milvexian (or a pharmaceutically acceptable salt or solvate thereof) and one or more pharmaceutically acceptable excipients.
Aspect 52. The method of aspect 51, wherein the solid oral pharmaceutical composition is an immediate release tablet.
Aspect 53. The method of aspect 52, wherein the immediate release tablet has a disintegration time in water of less than 20 seconds at 37° C.
Aspect 54. The method of any one of aspects 30-53, wherein the administration results in a milvexian plasma half-life ranging from about 13 hours to about 16 hours.
Aspect 55. The method of any one of aspects 30-54, wherein the administration results in a milvexian plasma half-life ranging from about 13 hours to about 16 hours during repeat dosing.
Aspect 56. The method of any one of aspects 30-55, wherein the administration results in milvexian plasma concentrations reaching steady-state at about 3 days to 6 days.
Aspect 57. The method of any one of aspects 30-56, wherein the pharmaceutical composition comprising milvexian (or a pharmaceutically acceptable salt or solvate thereof) is administered without regard to the timing of food intake.
Aspect 58. The method of any one aspects 30-57, wherein the patient is unable to swallow a tablet dosage form.
Aspect 59. The method of any one aspects 30-58, wherein the pharmaceutical composition is dispersed in an aqueous medium to form an aqueous dispersion.
Aspect 60. The method of aspect 59, wherein the aqueous medium is water, saline, phosphate buffer, vegetable juice, or fruit juice, including, for example, apple sauce.
Aspect 61. The method of aspect 59 or aspect 60, wherein the aqueous dispersion is administered to the human patient via a nasogastric tube or spoon.
Aspect 62. The method of any one of aspects 59-61, wherein the milvexian is administered orally as an aqueous dispersion by dispersing the pharmaceutical composition in an aqueous medium in less than 60 seconds at 37° C.
Aspect 63. The method of any one of any one of aspects 30-62, wherein the composition comprising milvexian (or a pharmaceutically acceptable salt or solvate thereof) is administered twice daily for at least 13 weeks.
Aspect 64. The method of any one of any one of aspects 30-63, wherein the composition comprising milvexian (or a pharmaceutically acceptable salt or solvate thereof) is administered once daily in the morning and once daily in the evening.
Aspect 65. The method of aspect 64, wherein the morning administration is made at the same time each day and the evening administration is made at the same time each day.
Aspect 66. The method of any one of aspects 30-65, wherein the administration reduces FXI clotting activity in the patient by about 27% to about 64% relative to the baseline FXI clotting activity.
Aspect 67. The method of any one of any one of aspects 30-66, wherein the administration results in a prolongation of activated partial thromboplastin time (aPTT) in the patient with a ratio to baseline of 2.1 to 2.6.
Aspect 68. A method of preventing stroke in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 69. A method of preventing non-central nervous system (CNS) systemic embolism events in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 70. A method of preventing stroke in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 71. A method of preventing non-central nervous system (CNS) systemic embolism events in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 72. A method of preventing stroke in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 73. A method of preventing non-central nervous system (CNS) systemic embolism events in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 74. A method of preventing stroke in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 75. A method of preventing non-central nervous system (CNS) systemic embolism events in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 76. The method of any one of aspects 68-75, wherein the patient has a history of atrial fibrillation or flutter, and also has concomitant coronary artery disease (CAD) and/or peripheral artery disease (PAD).
Aspect 77. The method of aspect 76, wherein the patient has a history of atrial fibrillation or flutter, and also has concomitant coronary artery disease (CAD).
Aspect 78. The method of aspect 76, wherein the patient has a history of atrial fibrillation or flutter, and also has concomitant peripheral artery disease (PAD).
Aspect 79. The method of any one of aspects 68-78, wherein the immediate-release film-coated tablet has a disintegration time in water of less than 20 seconds at 37° C.
Aspect 80. The method of any one of aspects 68-79, wherein the administration results in a milvexian plasma half-life ranging from about 13 hours to about 16 hours.
Aspect 81. The method of any one of aspects 68-80, wherein the administration results in a milvexian plasma half-life ranging from about 13 hours to about 16 hours during repeat dosing.
Aspect 82. The method of any one of aspects 68-81, wherein the administration results in milvexian plasma concentrations reaching steady-state at about 3 days to 6 days.
Aspect 83. The method of any one of aspects 68-82, wherein the immediate-release film-coated tablet is administered without regard to the timing of food intake.
Aspect 84. The method of any one aspects 68-83, wherein the patient is unable to swallow a tablet dosage form.
Aspect 85. The method of any one aspects 68-84, wherein the immediate-release film-coated tablet is dispersed in an aqueous medium to form an aqueous dispersion.
Aspect 86. The method of aspect 85, wherein the aqueous medium is water, saline, phosphate buffer, vegetable juice, or fruit juice, including, for example, apple sauce.
Aspect 87. The method of aspect 85 or aspect 86, wherein the aqueous dispersion is administered to the human patient via a nasogastric tube or spoon.
Aspect 88. The method of any one of aspects 68-87, wherein the milvexian is administered orally as an aqueous dispersion by dispersing the immediate-release film-coated tablet in an aqueous medium in less than 60 seconds at 37° C.
Aspect 89. The method of any one of any one of aspects 68-88, wherein the immediate-release film-coated tablet is administered twice daily for at least 13 weeks.
Aspect 90. The method of any one of any one of aspects 68-89, wherein the immediate-release film-coated tablet is administered once daily in the morning and once daily in the evening.
Aspect 91. The method of aspect 90, wherein the morning administration is made at the same time each day and the evening administration is made at the same time each day.
Aspect 92. The method of any one of aspects 68-91, wherein the administration reduces FXI clotting activity in the patient by about 27% to about 64% relative to the baseline FXI clotting activity.
Aspect 93. The method of any one of any one of aspects 68-91, wherein the administration results in a prolongation of activated partial thromboplastin time (aPTT) in the patient with a ratio to baseline of 2.1 to 2.6.
Aspect 94. The method of any one of the preceding aspects, wherein the method further comprises administering a standard-of-care antiplatelet therapy to the patient.
Aspect 95. The method of aspect 94, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.
Aspect 96. The method of any one of aspects 94 and 95, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.
Aspect 97. The method of any one of aspects 94-97, wherein the standard-of-care antiplatelet therapy is selected from aspirin, clopidogrel, ticagrelor, or prasugrel.
Aspect 98. The method of any one of aspects 94-97, wherein the standard-of-care antiplatelet therapy is selected from clopidogrel, ticagrelor, or prasugrel.
Aspect 99. The method of any one of aspects 94-98, wherein the standard-of-care antiplatelet therapy comprises a single antiplatelet therapy (SAPT).
Aspect 100. The method of aspect 99, wherein the single antiplatelet therapy comprises a aspirin, or a P2Y12 inhibitor selected from clopidogrel, ticagrelor, or prasugrel.
Aspect 101. The method of any one of aspects 94-98, wherein the standard-of-care antiplatelet therapy comprises a dual antiplatelet therapy (DAPT).
Aspect 102. The method of aspect 101, wherein the dual antiplatelet therapy comprises aspirin and a P2Y12 inhibitor selected from clopidogrel, ticagrelor, or prasugrel.
Aspect 103. The method of any one of aspects 101 and 102, wherein the dual antiplatelet therapy comprises a aspirin and a P2Y12 inhibitor selected from clopidogrel, ticagrelor, or prasugrel.
Aspect 104. The method of any one of aspects 101-103, wherein the dual antiplatelet therapy comprises a aspirin and clopidogrel.
Aspect 105. The method of any one of aspects 101-104, wherein the dual antiplatelet therapy (DAPT) is administered for 21 days, followed by single antiplatelet therapy (SAPT) thereafter.
Aspect 106. The method of any one of aspects 101-104, wherein the dual antiplatelet therapy (ie., aspirin and P2Y12 inhibitor) is administered for more than 90 days (with or without de-escalation to SAPT).
Aspect 107. Milvexian for use in preventing one or more of stroke and non-central nervous system (CNS) systemic embolism events in a human patient with a history of atrial fibrillation or flutter, wherein the method comprises administering to the human patient a pharmaceutical composition comprising about 50 mg, about 75.0 mg, about 87.5 mg, or about 100 mg of milvexian (or a pharmaceutically acceptable salt or solvate thereof, on a milvexian basis); wherein the pharmaceutical composition is administered twice daily.
Aspect 108. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg, about 75.0 mg, about 87.5 mg, or 100 mg of milvexian (or a pharmaceutically acceptable salt or solvate thereof, on a milvexian basis); wherein the composition is administered twice daily.
Aspect 109. The milvexian for use of aspect 108, wherein the adverse cardiovascular event is cardiovascular death.
Aspect 110. The milvexian for use of any one of aspects 107-109, wherein the patient has a history of atrial fibrillation or flutter, and also has concomitant coronary artery disease (CAD) and/or peripheral artery disease (PAD).
Aspect 111. The milvexian for use of any one of aspects 107-110, wherein the pharmaceutical composition comprises 100 mg of milvexian (or a pharmaceutically acceptable salt or solvate thereof, on a milvexian basis).
Aspect 112. The milvexian for use of any one of aspects 107-110, wherein the pharmaceutical composition comprises 87.5 mg of milvexian (or a pharmaceutically acceptable salt or solvate thereof, on a milvexian basis).
Aspect 113. The milvexian for use of any one of aspects 107-110, wherein the pharmaceutical composition comprises 75 mg of milvexian (or a pharmaceutically acceptable salt or solvate thereof, on a milvexian basis).
Aspect 114. The milvexian for use of any one of aspects 107-110, wherein the pharmaceutical composition comprises 50 mg of milvexian (or a pharmaceutically acceptable salt or solvate thereof, on a milvexian basis).
Aspect 115. The milvexian for use of any one of aspects 107-114, wherein the pharmaceutical composition is a solid oral pharmaceutical composition comprising milvexian (or a pharmaceutically acceptable salt or solvate thereof) and one or more pharmaceutically acceptable excipients.
Aspect 116. The milvexian for use of aspect 115, wherein the solid oral pharmaceutical composition is an immediate release tablet.
Aspect 117. The milvexian for use of aspect 116, wherein the immediate release tablet has a disintegration time in water of less than 20 seconds at 37° C.
Aspect 118. The milvexian for use of any one of aspects 107-117, wherein the administration results in a milvexian plasma half-life ranging from about 13 hours to about 16 hours.
Aspect 119. The milvexian for use of any one of aspects 107-118, wherein the administration results in a milvexian plasma half-life ranging from about 13 hours to about 16 hours during repeat dosing.
Aspect 120. The milvexian for use of any one of aspects 107-119, wherein the administration results in milvexian plasma concentrations reaching steady-state at about 3 days to 6 days.
Aspect 121. The milvexian for use of any one of aspects 107-120, wherein the pharmaceutical composition comprising milvexian (or a pharmaceutically acceptable salt or solvate thereof) is administered without regard to the timing of food intake.
Aspect 122. The milvexian for use of any one of aspects 107-121, wherein the composition comprising milvexian (or a pharmaceutically acceptable salt or solvate thereof) is administered twice daily for at least 13 weeks.
Aspect 123. The milvexian for use of any one of aspects 107-122, wherein the composition comprising milvexian (or a pharmaceutically acceptable salt or solvate thereof) is administered once daily in the morning and once daily in the evening.
Aspect 124. The milvexian for use of any one of aspects 107-123, wherein the morning administration is made at the same time each day and the evening administration is made at the same time each day.
Aspect 125. Milvexian for use in preventing one or more of stroke and non-central nervous system (CNS) systemic embolism events in a patient with a history of atrial fibrillation or flutter, wherein the method comprising administering to the patient an immediate release film coated tablet comprising about 50 mg, about 75.0 mg, about 87.5 mg, or 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (xvi) age between 65 and 74 years;
      (xvii) hypertension;
      (xviii) diabetes mellitus;
      (xix) history of coronary artery disease (CAD) (including post myocardial infarction or acute coronary syndrome event with or without percutaneous coronary intervention (PCI)) or peripheral artery disease (PAD); and
      (xx) history of heart failure.
Aspect 126. Milvexian for use in preventing one or more adverse cardiovascular events in a patient with a history of atrial fibrillation or flutter, wherein each event is selected from the group consisting of cardiovascular death, myocardial infarction, stroke, and non-central nervous system (CNS) systemic embolism said method comprising administering to the patient an immediate release film-coated tablet comprising about 50 mg, about 75.0 mg, about 87.5 mg, or 100 mg of milvexian, or a pharmaceutically acceptable salt thereof, and one or more pharmaceutically acceptable excipient,
   wherein the immediate release film-coated tablet is administered twice a day with or without a meal,
   wherein the patient is at least 18 years old;
   wherein the patient further has one or more of Category (A) risk factor selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 127. The milvexian for use of aspect 126, wherein the adverse cardiovascular event is cardiovascular death.
Aspect 128. The milvexian for use of any one of aspects 125-127, wherein immediate release film-coated tablet has a disintegration time in water of less than 20 seconds at 37° C.
Aspect 129. The milvexian for use of any one of aspects 125-128, wherein the administered milvexian has a plasma half-life of 13-16 hours.
Aspect 130. The milvexian for use of any one of aspects 125-129, wherein the administered milvexian has a plasma half-life of 13-16 hours during repeat dosing.
Aspect 131. The milvexian for use of any one of aspects 125-130, wherein the composition comprising milvexian (or a pharmaceutically acceptable salt or solvate thereof) is administered twice daily for at least 13 weeks.
Aspect 132. The milvexian for use of any one of aspects 105-131, wherein the composition comprising milvexian (or a pharmaceutically acceptable salt or solvate thereof) is administered once daily in the morning and once daily in the evening.
Aspect 133. The milvexian for use of aspect 132, wherein the morning administration is made at the same time each day and the evening administration is made at the same time each day.
Aspect 134. The milvexian for use of any one of aspects 107-133, wherein the administration reduces FXI clotting activity in the patient by about 27% to about 64% relative to the baseline FXI clotting activity.
Aspect 135. The milvexian for use of any one of aspects 107-134, wherein the administration results in a prolongation of activated partial thromboplastin time (aPTT) in the patient with a ratio to baseline of 2.1 to 2.6.
Aspect 136. Milvexian for use in preventing stroke in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.
Aspect 137. Milvexian for use in preventing stroke in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.
Aspect 138. Milvexian for use in preventing stroke in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.
Aspect 139. Milvexian for use in preventing stroke in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 140. Milvexian for use in preventing one or more non-central nervous system (CNS) systemic embolism events in a human patient with a history of atrial fibrillation or flutter, wherein the method comprises administering to the human patient a pharmaceutical composition comprising about 50 mg of milvexian (or a pharmaceutically acceptable salt or solvate thereof, on a milvexian basis), wherein the pharmaceutical composition is administered twice daily.
Aspect 141. Milvexian for use in preventing one or more non-central nervous system (CNS) systemic embolism events in a human patient with a history of atrial fibrillation or flutter, wherein the method comprises administering to the human patient a pharmaceutical composition comprising about 75 mg of milvexian (or a pharmaceutically acceptable salt or solvate thereof, on a milvexian basis), wherein the pharmaceutical composition is administered twice daily.
Aspect 142. Milvexian for use in preventing one or more non-central nervous system (CNS) systemic embolism events in a human patient with a history of atrial fibrillation or flutter, wherein the method comprises administering to the human patient a pharmaceutical composition comprising about 87.5 mg of milvexian (or a pharmaceutically acceptable salt or solvate thereof, on a milvexian basis), wherein the pharmaceutical composition is administered twice daily.
Aspect 143. Milvexian for use in preventing one or more non-central nervous system (CNS) systemic embolism events in a human patient with a history of atrial fibrillation or flutter, wherein the method comprises administering to the human patient a pharmaceutical composition comprising about 100 mg of milvexian (or a pharmaceutically acceptable salt or solvate thereof, on a milvexian basis), wherein the pharmaceutical composition is administered twice daily.
Aspect 144. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg of milvexian (or a pharmaceutically acceptable salt or solvate thereof, on a milvexian basis), wherein the composition is administered twice daily.
Aspect 145. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 75 mg of milvexian (or a pharmaceutically acceptable salt or solvate thereof, on a milvexian basis), wherein the composition is administered twice daily.
Aspect 146. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 87.5 mg of milvexian (or a pharmaceutically acceptable salt or solvate thereof, on a milvexian basis), wherein the composition is administered twice daily.
Aspect 147. Milvexian for use in preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian (or a pharmaceutically acceptable salt or solvate thereof, on a milvexian basis); wherein the composition is administered twice daily.
Aspect 148. The milvexian for use of any one of aspects 144-147, wherein the adverse cardiovascular event is cardiovascular death.
Aspect 149. The milvexian for use of any one of aspects 144-147, wherein the adverse cardiovascular event is myocardial infarction.
Aspect 150. The milvexian for use of any one of aspects 144-147, wherein the adverse cardiovascular event is stroke.
Aspect 151. The milvexian for use of any one of aspects 144-147, wherein the adverse cardiovascular event is non-central nervous system (CNS) systemic embolism.
Aspect 152. The milvexian for use of any one of aspects 136-151, wherein the patient has a history of atrial fibrillation or flutter, and also has concomitant coronary artery disease (CAD) and/or peripheral artery disease (PAD).
Aspect 153. The milvexian for use of aspect 152, wherein the patient has a history of atrial fibrillation or flutter, and also has concomitant coronary artery disease (CAD).
Aspect 154. The milvexian for use of aspect 152, wherein the patient has a history of atrial fibrillation or flutter, and also has concomitant peripheral artery disease (PAD).
Aspect 155. The milvexian for use of any one of aspects 136-154, wherein the pharmaceutical composition is a solid oral pharmaceutical composition comprising milvexian (or a pharmaceutically acceptable salt or solvate thereof) and one or more pharmaceutically acceptable excipients.
Aspect 156. The milvexian for use of aspect 155, wherein the solid oral pharmaceutical composition is an immediate release tablet.
Aspect 157. The milvexian for use of aspect 156, wherein the immediate release tablet has a disintegration time in water of less than 20 seconds at 37° C.
Aspect 158. The milvexian for use of any one of aspects 136-157, wherein the administration results in a milvexian plasma half-life ranging from about 13 hours to about 16 hours.
Aspect 159. The milvexian for use of any one of aspects 136-158, wherein the administration results in a milvexian plasma half-life ranging from about 13 hours to about 16 hours during repeat dosing.
Aspect 160. The milvexian for use of any one of aspects 136-159, wherein the administration results in milvexian plasma concentrations reaching steady-state at about 3 days to 6 days.
Aspect 161. The milvexian for use of any one of aspects 136-160, wherein the pharmaceutical composition comprising milvexian (or a pharmaceutically acceptable salt or solvate thereof) is administered without regard to the timing of food intake.
Aspect 162. The milvexian for use of any one aspects 136-161, wherein the patient is unable to swallow a tablet dosage form.
Aspect 163. The milvexian for use of any one aspects 136-162, wherein the pharmaceutical composition is dispersed in an aqueous medium to form an aqueous dispersion.
Aspect 164. The milvexian for use of aspect 163, wherein the aqueous medium is water, saline, phosphate buffer, vegetable juice, or fruit juice, including, for example, apple sauce.
Aspect 165. The milvexian for use of aspect 163 or aspect 164, wherein the aqueous dispersion is administered to the human patient via a nasogastric tube or spoon.
Aspect 166. The milvexian for use of any one of aspects 163-165, wherein the milvexian is administered orally as an aqueous dispersion by dispersing the pharmaceutical composition in an aqueous medium in less than 60 seconds at 37° C.
Aspect 167. The milvexian for use of any one of any one of aspects 136-166, wherein the composition comprising milvexian (or a pharmaceutically acceptable salt or solvate thereof) is administered twice daily for at least 13 weeks.
Aspect 168. The milvexian for use of any one of any one of aspects 136-167, wherein the composition comprising milvexian (or a pharmaceutically acceptable salt or solvate thereof) is administered once daily in the morning and once daily in the evening.
Aspect 169. The milvexian for use of aspect 168, wherein the morning administration is made at the same time each day and the evening administration is made at the same time each day.
Aspect 170. The milvexian for use of any one of aspects 136-169, wherein the administration reduces FXI clotting activity in the patient by about 27% to about 64% relative to the baseline FXI clotting activity.
Aspect 171. The milvexian for use of any one of any one of aspects 136-170, wherein the administration results in a prolongation of activated partial thromboplastin time (aPTT) in the patient with a ratio to baseline of 2.1 to 2.6.
Aspect 172. Milvexian for use in preventing stroke in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 173. Milvexian for use in preventing non-central nervous system (CNS) systemic embolism events in a patient with a history of atrial fibrillation or flutter, wherein the method comprising administering to the patient an immediate release film coated tablet comprising about 50 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 174. Milvexian for use in preventing stroke in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 175. Milvexian for use in preventing non-central nervous system (CNS) systemic embolism events in a patient with a history of atrial fibrillation or flutter, wherein the method comprising administering to the patient an immediate release film coated tablet comprising about 75 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 176. Milvexian for use in preventing stroke in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 177. Milvexian for use in preventing non-central nervous system (CNS) systemic embolism events in a patient with a history of atrial fibrillation or flutter, wherein the method comprising administering to the patient an immediate release film coated tablet comprising about 87.5 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 178. Milvexian for use in preventing stroke in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 179. Milvexian for use in preventing non-central nervous system (CNS) systemic embolism events in a patient with a history of atrial fibrillation or flutter, wherein the method comprising administering to the patient an immediate release film coated tablet comprising about 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 180. The milvexian for use of any one of aspects 172-179, wherein the patient has a history of atrial fibrillation or flutter, and also has concomitant coronary artery disease (CAD) and/or peripheral artery disease (PAD).
Aspect 181. The milvexian for use of aspect 180, wherein the patient has a history of atrial fibrillation or flutter, and also has concomitant coronary artery disease (CAD).
Aspect 182. The milvexian for use of aspect 180, wherein the patient has a history of atrial fibrillation or flutter, and also has concomitant peripheral artery disease (PAD).
Aspect 183. The milvexian for use of any one of aspects 172-182, wherein the immediate-release film-coated tablet has a disintegration time in water of less than 20 seconds at 37° C.
Aspect 184. The milvexian for use of any one of aspects 172-183, wherein the administration results in a milvexian plasma half-life ranging from about 13 hours to about 16 hours.
Aspect 185. The milvexian for use of any one of aspects 172-184, wherein the administration results in a milvexian plasma half-life ranging from about 13 hours to about 16 hours during repeat dosing.
Aspect 186. The milvexian for use of any one of aspects 172-185, wherein the administration results in milvexian plasma concentrations reaching steady-state at about 3 days to 6 days.
Aspect 187. The milvexian for use of any one of aspects 172-186, wherein the immediate-release film-coated tablet is administered without regard to the timing of food intake.
Aspect 188. The milvexian for use of any one aspects 172-187, wherein the patient is unable to swallow a tablet dosage form.
Aspect 189. The milvexian for use of any one aspects 172-188, wherein the immediate-release film-coated tablet is dispersed in an aqueous medium to form an aqueous dispersion.
Aspect 190. The milvexian for use of aspect 189, wherein the aqueous medium is water, saline, phosphate buffer, vegetable juice, or fruit juice, including, for example, apple sauce.
Aspect 191. The milvexian for use of aspect 189 or aspect 190, wherein the aqueous dispersion is administered to the human patient via a nasogastric tube or spoon.
Aspect 192. The milvexian for use of any one of aspects 172-191, wherein the milvexian is administered orally as an aqueous dispersion by dispersing the immediate-release film-coated tablet in an aqueous medium in less than 60 seconds at 37° C.
Aspect 193. The milvexian for use of any one of any one of aspects 172-192, wherein the immediate-release film-coated tablet is administered twice daily for at least 13 weeks.
Aspect 194. The milvexian for use of any one of any one of aspects 172-193, wherein the immediate-release film-coated tablet is administered once daily in the morning and once daily in the evening.
Aspect 195. The milvexian for use of aspect 194, wherein the morning administration is made at the same time each day and the evening administration is made at the same time each day.
Aspect 196. The milvexian for use of any one of aspects 172-195, wherein the administration reduces FXI clotting activity in the patient by about 27% to about 64% relative to the baseline FXI clotting activity.
Aspect 197. The milvexian for use of any one of any one of aspects 172-196, wherein the administration results in a prolongation of activated partial thromboplastin time (aPTT) in the patient with a ratio to baseline of 2.1 to 2.6.
Aspect 198. The milvexian for use of any one of aspects 107-197, wherein the use further comprises administering a standard-of-care antiplatelet therapy to the patient.
Aspect 199. The milvexian for use of aspect 198, wherein, the standard-of-care antiplatelet therapy is selected from aspirin, adenosine diphosphate (ADP) receptor inhibitors; adenosine reuptake inhibitors; glycoprotein platelet inhibitors; phosphodiesterase inhibitors; or protease-activated receptor (PAR-1) antagonist.
Aspect 200. The milvexian for use of aspects 198 and 199, wherein the standard-of-care antiplatelet therapy is selected from aspirin, clopidogrel, ticagrelor, ticlopidine, prasugrel, dipyridamole, abciximab, eptifibatide, tirofiban, cilostazol, or vorapaxar.
Aspect 201. The milvexian for use of any one of aspects 198-200, wherein the standard-of-care antiplatelet therapy is selected from aspirin, clopidogrel, ticagrelor, or prasugrel.
Aspect 202. The milvexian for use of any one of aspects 198-201, wherein the standard-of-care antiplatelet therapy is selected from clopidogrel, ticagrelor, or prasugrel.
Aspect 203. The milvexian for use of any one of aspects 198-202, wherein the standard-of-care antiplatelet therapy comprises a single antiplatelet therapy (SAPT).
Aspect 204. The milvexian for use of aspect 203, wherein the single antiplatelet therapy comprises a aspirin, or a P2Y12 inhibitor selected from clopidogrel, ticagrelor, or prasugrel.
Aspect 205. The milvexian for use of any one of aspects 198-204, wherein the standard-of-care antiplatelet therapy comprises a dual antiplatelet therapy (DAPT).
Aspect 206. The milvexian for use of aspect 205, wherein the dual antiplatelet therapy comprises aspirin and a P2Y12 inhibitor selected from clopidogrel, ticagrelor, or prasugrel.
Aspect 207. The milvexian for use of any one of aspects 205 and 206, wherein the dual antiplatelet therapy comprises a aspirin and a P2Y12 inhibitor selected from clopidogrel, ticagrelor, or prasugrel.
Aspect 208. The milvexian for use of any one of aspects 205-207, wherein the dual antiplatelet therapy comprises a aspirin and clopidogrel.
Aspect 209. The milvexian for use of any one of aspects 205-208, wherein the dual antiplatelet therapy (DAPT) is administered for 21 days, followed by single antiplatelet therapy (SAPT) thereafter.
Aspect 210. The milvexian for use of any one of aspects 205-208, wherein the dual antiplatelet therapy (ie., aspirin and P2Y12 inhibitor) is administered for more than 90 days (with or without de-escalation to SAPT).
Aspect 211. A method for preventing one or more of stroke and non-central nervous system (CNS) systemic embolism events in a human patient with a history of atrial fibrillation or flutter, wherein the method comprises administering to the human patient a pharmaceutical composition comprising about 50 mg of milvexian, or a pharmaceutically acceptable salt thereof; wherein the pharmaceutical composition is administered twice daily.
Aspect 212. A method for preventing one or more of stroke and non-central nervous system (CNS) systemic embolism events in a human patient with a history of atrial fibrillation or flutter, wherein the method comprises administering to the human patient a pharmaceutical composition comprising about 100 mg of milvexian, or a pharmaceutically acceptable salt thereof, wherein the pharmaceutical composition is administered twice daily.
Aspect 213. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg of milvexian, or a pharmaceutically acceptable salt thereof; wherein the composition is administered twice daily.
Aspect 214. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.
Aspect 215. A method for reducing the risk of one or more of stroke and non-central nervous system (CNS) systemic embolism events in a human patient with a history of atrial fibrillation or flutter, wherein the method comprises administering to the human patient a pharmaceutical composition comprising about 50 mg of milvexian, or a pharmaceutically acceptable salt thereof; wherein the pharmaceutical composition is administered twice daily.
Aspect 216. A method for reducing the risk of one or more of stroke and non-central nervous system (CNS) systemic embolism events in a human patient with a history of atrial fibrillation or flutter, wherein the method comprises administering to the human patient a pharmaceutical composition comprising about 100 mg of milvexian, or a pharmaceutically acceptable salt thereof; wherein the pharmaceutical composition is administered twice daily.
Aspect 217. A method for reducing the risk of one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 50 mg of milvexian, or a pharmaceutically acceptable salt thereof; wherein the composition is administered twice daily.
Aspect 218. A method for reducing the risk of one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising about 100 mg of milvexian, or a pharmaceutically acceptable salt thereof; wherein the composition is administered twice daily.
Aspect 219. The method of any one of aspects 211-218, wherein the reduction of risk is measured by hazard ratio (HR) at a value ranging from about 0.5 to about 1.1.
Aspect 220. The method of aspect 219, wherein HR has a value ranging from about 0.65 to about 0.86.
Aspect 221. The method of aspect 220, wherein HR has a value of about 0.84.

### Aspect Set #3

Aspect 1. A method for preventing one or more of stroke and non-central nervous system (CNS) systemic embolism events in a human patient with a history of atrial fibrillation or flutter, wherein the method comprises administering to the human patient a pharmaceutical composition comprising 50 mg, or 100 mg, of milvexian (or a pharmaceutically acceptable salt or solvate thereof, on a milvexian basis); wherein the pharmaceutical composition is administered twice daily.
Aspect 2. A method for preventing one or more of adverse cardiovascular events in a patient with a history of atrial fibrillation or flutter, wherein each event is selected from the group consisting of cardiovascular death, myocardial infarction, stroke, and non-central nervous system (CNS) systemic embolism, wherein the method comprises administering to the human patient a pharmaceutical composition comprising 50 mg, or 100 mg, of milvexian (or a pharmaceutically acceptable salt or solvate thereof, on a milvexian basis); wherein the composition is administered twice daily.
Aspect 3. The method of aspect 2, wherein the adverse cardiovascular event is cardiovascular death.
Aspect 4. The method of any one of aspects 1-3, wherein the patient has a history of atrial fibrillation or flutter, and also has concomitant coronary artery disease (CAD) and/or peripheral artery disease (PAD).
Aspect 5. The method of any one of aspects 1-4, wherein the pharmaceutical composition comprises 100 mg of milvexian (or a pharmaceutically acceptable salt or solvate thereof, on a milvexian basis).
Aspect 6. The method of any one of aspects 1-4, wherein the pharmaceutical composition comprises 50 mg of milvexian (or a pharmaceutically acceptable salt or solvate thereof, on a milvexian basis).
Aspect 7. The method of any one of aspects 1-6, wherein the pharmaceutical composition is a solid oral pharmaceutical composition comprising milvexian (or a pharmaceutically acceptable salt or solvate thereof) and one or more pharmaceutically acceptable excipients.
Aspect 8. The method of aspect 7, wherein the solid oral pharmaceutical composition is an immediate release tablet.
Aspect 9. The method of aspect 8, wherein the immediate release tablet has a disintegration time in water of less than 20 seconds at 37° C.
Aspect 10. The method of any one of aspects 5-9, wherein the administration results in a milvexian plasma half-life ranging from about 13 hours to about 16 hours.
Aspect 11. The method of any one of the preceding aspects, wherein the administration results in milvexian plasma concentrations reaching steady-state at about 3 days to 6 days.
Aspect 12. The method of any one of the preceding aspects, wherein the pharmaceutical composition comprising milvexian (or a pharmaceutically acceptable salt or solvate thereof) is administered without regard to the timing of food intake.
Aspect 13. A method of preventing one or more of stroke and non-central nervous system (CNS) systemic embolism events in a patient with a history of atrial fibrillation or flutter, wherein the method comprising administering to the patient an immediate release film coated tablet comprising 50 mg or 100 mg of milvexian or a pharmaceutically acceptable salt thereof and one or more pharmaceutically acceptable excipients,
   wherein the immediate release film-coated tablet is administered twice a day with or without a meal,
   wherein the patient is at least 18 years old;
   wherein the patient further has one or more of Category (A) risk factors selected from (i) age greater than or equal to 75 years or (ii) history of ischemic stroke or silent brain infarct; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 14. A method of preventing one or more adverse cardiovascular events in a patient with a history of atrial fibrillation or flutter, wherein each event is selected from the group consisting of cardiovascular death, myocardial infarction, stroke, and non-central nervous system (CNS) systemic embolism said method comprising administering to the patient an immediate release film-coated tablet comprising 50 mg or 100 mg of milvexian, or a pharmaceutically acceptable salt thereof, and one or more pharmaceutically acceptable excipient,
   wherein the immediate release film-coated tablet is administered twice a day with or without a meal,
   wherein the patient is at least 18 years old;
   wherein the patient further has one or more of Category (A) risk factor selected from (i) age greater than or equal to 75 years or (ii) history of ischemic stroke or silent brain infarct; or two or more of concomitant or comorbid Category (B) cardiovascular risk factor selected from the group consisting of:
      (i) age between 65 and 74 years;
      (ii) hypertension;
      (iii) diabetes mellitus;
      (iv) history of coronary artery disease (CAD) (including post myocardial infarction or acute coronary syndrome event with or without percutaneous coronary intervention (PCI)) or peripheral artery disease (PAD); and
      (v) history of heart failure.
Aspect 15. The method of aspect 14, wherein the adverse cardiovascular event is cardiovascular death.
Aspect 16. The method of any one of aspects 13-15, wherein immediate release film-coated tablet has a disintegration time in water of less than 20 seconds at 37° C.
Aspect 17. The method of any one of aspects 13-16, wherein the administered milvexian has a plasma half-life of 13-16 hours.
Aspect 18. The method of any one of the preceding aspects, wherein the administration reduces FXI clotting activity in the patient by about 27% to about 64% relative to the baseline FXI clotting activity.
Aspect 19. The method of any one of the preceding aspects, wherein the administration results in a prolongation of activated partial thromboplastin time (aPTT) in the patient with a ratio to baseline of 2.1 to 2.6.
Aspect 20. The method of any one of the preceding aspects, wherein the composition comprising milvexian (or a pharmaceutically acceptable salt or solvate thereof) is administered twice daily for at least 13 weeks.
Aspect 21. The method of any one of the preceding aspects, wherein the composition comprising milvexian (or a pharmaceutically acceptable salt or solvate thereof) is administered once daily in the morning and once daily in the evening.
Aspect 22. The method of aspect 21, wherein the morning administration is made at the same time each day and the evening administration is made at the same time each day.

### EXAMPLES

### Example 1. A Phase 3, Randomized, Double-Blind, Double-Dummy, Parallel Group, Active-Controlled Study to Evaluate the Efficacy and Safety of Milvexian, an Oral Factor XIa Inhibitor, Versus Apixaban in Participants with Atrial Fibrillation

This is a Phase 3, prospective, randomized, double-blind, parallel-group, active-controlled, multicenter, event driven study comparing the efficacy and safety of milvexian to apixaban for the prevention of stroke and non-CNS systemic embolism in participants with atrial fibrillation. The primary hypothesis is that milvexian is non-inferior to apixaban in reducing the risk of the composite endpoint of stroke and non-CNS systemic embolism in participants with atrial fibrillation.

### Study Population

Approximately 15,500 participants with AF are randomized to reach the pre-specified number of required endpoint events.

### Inclusion Criteria

Each potential participant must satisfy all of the following criteria to be enrolled in the study:

### Age

1. Minimum age of 18 years (or the legal age of consent in the jurisdiction in which the study is taking place)

### Type of Participant and Disease Characteristics

2. Medically stable and appropriate for chronic antithrombotic treatment on the basis of physical examination, medical history, vital signs, and clinical laboratory tests performed as part of standard of care or at screening. Any abnormalities in physical examination, medical history or vital signs must be consistent with the underlying illness in the study population. If the results of the serum chemistry panel, hematology, or urinalysis are outside the normal reference ranges, the participant may be included only if the investigator judges the abnormalities or deviations from normal to be not clinically significant or to be appropriate and reasonable for the population under study. This determination must be recorded in the participant's source documents and initialed by the principal investigator or appropriately qualified designee.

3. Atrial fibrillation or flutter, paroxysmal or sustained not due to a reversible cause, and eligible to receive anticoagulation therapy:
i. Participants must have medical evidence of atrial fibrillation or flutter within 1 year of randomization. This could be obtained from a notation in the participant's record (eg, ECG, medical chart, hospital discharge summary).
ii. If electrical cardioversion or ablation is planned, the investigator plans to treat the patient with anticoagulation for the duration of the trial.

4. Participant must satisfy one or both of the following categories of risk factors (a or b):
a. One or more of the following risk factors:
   i. Age ≥75 years at the time of screening
   ii. History of a clinical symptomatic stroke or silent brain infarct of any type (ischemic, hemorrhagic, lacunar, or undetermined) or cerebral microbleeds (CMB):
   Ischemic stroke must be >7 days prior to first dose of study intervention. Hemorrhagic strokes, and hemorrhagic transformations must have occurred ≥3 months prior and are allowed per investigator discretion. A prior neurology consultation is recommended to ascertain if the patient is appropriate for anticoagulation. (See Exclusion Criterion 2 for history of subarachnoid hemorrhage, subdural hematoma, or spinal cord hemorrhage)
   Notes:
   CMBs are defined as rounded foci of <10 mm in size that appear hypointense and distinct from vascular flow voids, leptomeningeal hemosiderosis, or non-hemorrhagic subcortical mineralization on T2*-weighted MRI.
   Participants who received thrombolysis and/or mechanical thrombectomy for any indications other than stroke with or without stenting can be randomized, if they are at least 24-hours post-thrombolytic therapy/post-thrombectomy and have an INR ≤1.5 and aPTT ≤1.4 times the ULN prior to study randomization.
b. Two or more of the following risk factors:
   i. Age between 65 and 74 years, inclusive, at the time of screening
   ii. Hypertension (defined as use of antihypertensive medications within 6 months before the screening visit or persistent systolic blood pressure above 140 mmHg or diastolic blood pressure above 90 mmHg)
   iii. Diabetes mellitus (defined as a history of diabetes mellitus and current use of antidiabetic medications)
   iv. Atherosclerotic vascular disease meeting one or more of the following criteria:
      Lower extremity PAD defined as one or both of the following:
      a) a documented history of a resting ankle-brachial index (ABI) of <0.85 b) prior major vascular (non-traumatic) amputation (ankle or above), peripheral bypass, or peripheral percutaneous or surgical intervention for limb ischemia.
   AND/OR
   CAD defined as one of the following:
      a) a recent or past MI, excluding periprocedural or definite Type 2 MI
      b) history of coronary revascularization, either percutaneous (PCI) or surgical (CABG)
   v. Symptomatic heart failure (HF) defined as either: (a) History of hospitalization with HF as the primary cause (regardless of ejection fraction) or (b) left ventricular ejection fraction (LVEF) of <40% (most recent and assessed <1 year of randomization) regardless of history of heart failure hospitalization.

5. For participants receiving vitamin K antagonists (VKA) (e.g., Coumadin, Aldocumar, Coumadine, Jantoven, Marevan, Orfarin, Taro-Warfarin, Waran), the INR must be ≤2.0 at the time of randomization with the first dose of study intervention taken the same day.

### Weight

Not applicable.

### Sex and Contraceptive/Barrier Requirements

All female participants of childbearing potential must have a negative highly sensitive serum (β-human chorionic gonadotropin [β-hCG]) or urine test up to 2 days before the first dose of study intervention.

6. A female participant must agree not to be pregnant, breastfeeding, or planning to become pregnant while enrolled in this study until 4 days (five half-lives) after the last dose of study intervention.

7. A female participant must be
a. Not of childbearing potential; or
b. Of childbearing potential and practicing a highly effective method of contraception (failure rate of <1% per year when used consistently and correctly) and agrees to remain on a highly effective method until 4 days (5 half lives) after the last dose of study intervention—the end of relevant exposure.

8. A female participant using hormonal contraceptives should use an additional nonhormonal contraceptive method (above that required in the inclusion criterion 8b) until 4 days after the last dose (five half-lives) of study intervention—the end of relevant exposure.

### Exclusion Criteria

Any potential participant who meets any of the following criteria will be excluded from participating in the study:

### Medical Conditions

1. History of ischemic stroke, but only if within 7 days of stroke onset. An ischemic stroke with onset 8 or more days prior to randomization is permitted.

2. History of any of the following only if within 3 months of randomization: primary hemorrhagic stroke, hemorrhagic transformation of an ischemic stroke, subarachnoid hemorrhage, subdural hematoma, spinal cord hemorrhage.

3. A prior disabling stroke defined by a current mRS of ≥3. Refer to mRS scoring in Study Specific Materials.

4. Hemodynamically significant valve disease or those with valve disease that will potentially require surgical valve replacement during the study as determined by the investigator

5. Any condition other than AF that requires chronic anticoagulation at the discretion of the investigator and/or local guidelines, such as mechanical heart valves. However, participants with an indication for chronic antiplatelet therapy after placement of a bio-prosthetic nonmechanical valve (eg, transcatheter aortic valve replacement [TAVR]) do not satisfy this exclusion criterion and are eligible for study participation

6. Any condition that, in the opinion of the investigator, contraindicates anticoagulant therapy, or would, with anticoagulation, have an unacceptable risk of bleeding, or would interfere with the study endpoint assessments, eg, large cerebral infarct volume, active/recent major bleeding, known hereditary bleeding diathesis.

7. Known presence of atrial myxoma or left ventricular thrombus

8. Active endocarditis

9. Current active liver disease (eg, acute hepatitis, known cirrhosis), including participants receiving antiviral treatment for hepatitis

10. Require dialysis at time of randomization

11. Patients with eGFR <25 mL/min/1.73 m² at screening

12. History of any significant drug allergy (such as anaphylaxis, Stevens-Johnson Syndrome, toxic epidermal necrolysis, drug reaction with eosinophilia and systemic symptoms [DRESS]).

13. Hospitalized for acute heart failure at the time of randomization.

14. Known allergies, hypersensitivity, or intolerance to milvexian or its excipients.

15. Unable to swallow medications, as determined by the investigator at screening

### Prior/Concomitant Therapy

16. Planned use of any disallowed therapies including isoniazid (INH).

### Disallowed Therapies:

Isoniazid;
Aspirin>100 mg/day>7 days of consecutive use;
Concomitant use of omeprazole or esomeprazole with clopidogrel;
Additional chronic anticoagulants;
The concomitant use of a combined P-gp and strong CYP3A4 inhibitor (e.g., atazanavir, clarithromycin, itraconazole, ketoconazole, ritonavir, saquinavir) within 7 days of receiving study intervention and during the study;
The concomitant use of a combined P-gp and strong CYP3A4/5 inducer (e.g., carbamazepine, phenytoin, rifampin) within 7 days of receiving study intervention and during the study.

### Prior/Concurrent Clinical Study Experience

17. Received an investigational intervention or used an invasive investigational medical device within 4 weeks before the planned first dose of study intervention or is currently enrolled in an investigational interventional study.

### Diagnostic Assessments

18. Any of the following laboratory results, based on local laboratory, outside of the ranges specified below prior to randomization, confirmed by repeat:
a. Platelet count <50,000 mm³
b. ALT >3×ULN
c. Total bilirubin ≥1.5×ULN unless an alternative causative factor such as Gilbert's syndrome is identified
d. Hemoglobin <8.0 g/dL

### Other Exclusions

19. Any condition for which, in the opinion of the investigator, participation would not be in the best interest of the participant (eg, compromise the well-being) or that could prevent, limit, or confound the protocol-specified assessments or has a life expectancy of <12 months.

20. At the time of screening, any participant who will not consider following the study contact schedule, or not allow a contact to the participant, or to the designated family members or health care practitioner, to determine if any endpoint events and/or vital status, up until the end of the study should they prematurely discontinue study intervention or withdraw from study participation.

21. Participants who are incarcerated, including prisoners or subjects compulsorily detained for treatment of psychiatric disease.

22. Known current substance abuse that could impact study compliance

23. Employee of the investigator or study site, with direct involvement in the proposed study or other studies under the direction of that investigator or study site, as well as family members of the employees or the investigator.

### Dose Selection

The 100 mg twice daily dose regimen of milvexian chosen for this study was selected based on the totality of data, including data from the Phase 1 and Phase 2 studies and considerations of using an active comparator (apixaban) in the study design, and thrombotic event pathophysiology in patients with atrial fibrillation (AF; ie, primarily embolism from the left atrial appendage). Both the milvexian Phase 2 studies (AXIOMATICTKR and AXIOMATIC-SSP) primarily used twice daily dosing regimens. With a half-life of about 13 to 16 hours, repeating twice daily dosing provides a lower peak to trough ratio relative to the same total dose administered once a day according to the meta-analysis using applicant's proprietary algorithms (MBMA) and patient data. Madabushi et al., Review: Role of Model-Informed Drug Development Approaches in the Lifecycle of Drug Development and Regulatory Decision-Making, Pharmaceutical Research, 2022, vol. 39, pp. 1669-1680.

In the AXIOMATIC-TKR study (see Example 2), milvexian doses of 50-200 mg twice daily were superior to enoxaparin 40 mg once daily for the prevention of total venous thromboembolism (VTE) with only modest gains in efficacy for the 100 and 200 mg twice daily doses (enoxaparin 40 mg—21.4%, milvexian 50 mg—11.3%, 100 mg—9.0%, 200 mg—7.6%). Historical Phase 3 studies of VTE prevention in orthopedic surgery demonstrated that apixaban 2.5 mg twice daily and rivaroxaban 10 mg once daily were superior to enoxaparin 40 mg once daily. No fatal bleeding and no increases in symptomatic intracranial hemorrhage were observed with milvexian. No dose-response relationship was observed for bleeding events in either the AXIOMATIC-TKR or AXIOMATIC-SSP studies (see Examples 2 and 3, respectively), suggesting a lower bleeding liability for any dose of milvexian compared with Factor Xa inhibitors (i.e., both rivaroxaban and apixaban showed dose-related bleeding in Phase 2 orthopedic surgery studies and in combination with antiplatelets after an ACS).

In the development of currently approved direct oral anticoagulants (DOACs), studies of VTE prevention were used as a benchmark for predicting the prevention of cardioembolic events related to AF.

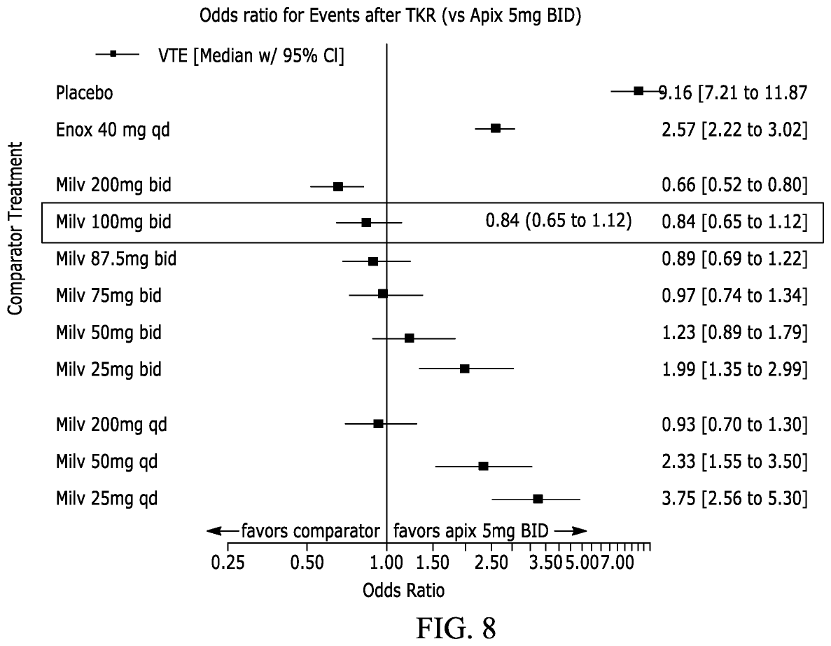

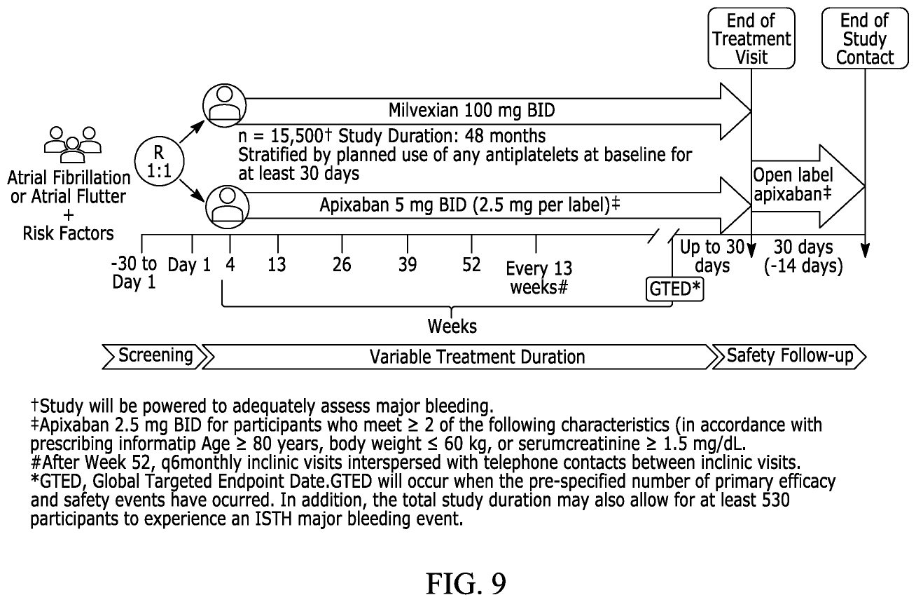

To guide selection of an efficacious dose of milvexian, a model based meta-analysis (MBMA) was performed. MBMA is a method used to integrate data from multiple studies using mathematical models to quantitatively describe the effect of treatments on trial outcomes. MBMA facilitates a quantitative comparison of the therapeutic landscape. A total 45 studies of VTE prevention, involving studies for currently approved DOACS, and milvexian AXIOMATIC-TKR and AXIOMATIC-SSP, were used to perform a MBMA. These included 54 treatment arms for 10 different treatments including placebo in a total of 5,434 trial participants. These randomized clinical trials were combined with the AXIOMATIC-TKR Phase 2 study results (applicant's proprietary information) to develop a non-linear mixed effect, Eₘₐₓ dose-response MBMA model (algorithm codes written by the applicant). Covariates included in the model were indication, geographic location, year of each trial, drug dosing frequency, and dosing information. Eₘₐₓ values were assumed to be shared across treatments for a single endpoint, while Ed₅₀ was estimated for each treatment separately. Random effects account for unexplained variability across trials by endpoint. This MBMA model provided a quantitative understanding of VTE rates for multiple milvexian doses compared against approved anti-thrombotic treatments (apixaban and Enoxaparin). Modeling results comparing milvexian against apixaban 5 mg BID as odds ratio of VTE are summarized in the forest plot in FIG. 8 where an odds ratio <1 favors milvexian. These results show that milvexian 100 mg twice daily (BID) exhibited an odds ratio of 0.84 [0.65, 1.12] (median [95% confidence interval, CI]) compared to apixaban 5 mg BID. The upper 95% CI bound of 1.12 falls within the boundary of non-inferiority for milvexian 100 mg BID against apixaban 5 mg BID. Lower doses were projected to have inferior efficacy as compared to apixaban 5 mg BID (See FIG. 8, odds ratio plot for milvexian at various BID doses vs apixaban at 5 mg BID in reducing VTE rates).

Considering both bleeding observations, efficacy projections, and other considerations, milvexian 100 mg twice daily was selected for confirming the primary hypothesis of the Phase 3 study.

### Study Drug

At randomization on Day 1, participants are randomly assigned in double blind manner in a 1:1 ratio to receive either milvexian 100 mg (twice daily) or apixaban 5.0 mg twice daily. The apixaban dose is 2.5 mg twice daily for participants with at least 2 of the following characteristics: age ≥80 years, body weight ≤60 kg, or serum creatinine ≥1.5 mg/dL. Study intervention may be taken without regard to food intake.

The milvexian dosage form is a film-coated, direct-compression immediate release tablet^{a} having the formulation described in Table 1 below:

TABLE 1
Film-coated Tablet of Milvexian at 100 mg Strength

| | Amount | | |
|---|---|---|---|
| | | Amount per dose | Amount per dose |
| Component | wt. % | (25 mg, Ex. 17) | (100 mg, Ex. 18) |
| SDP^{b} | 22.22 | 33.33 | 133.3 |
| Silicified Microcrystalline cellulose | 43.07 | 64.6 | 258.4 |
| Lactose monohydrate | 28.71 | 43.067 | 172.267 |
| Croscarmellose sodium | 5.0 | 7.5 | 30 |
| Magnesium stearate | 1.0 | 1.5 | 6 |
| Total Core Tablet Weight | 100 | 150 | 600 |
| OpadryQX 321A220063 Yellow^{c} | 3.0 | 4.5 | 18.0 |
| | | 154.5 | 618 |
| Tablet hardness | | Average 60N to | Average 140N to |
| | | 120N (Ph. | 220N (Ph. |
| | | Eur. 2.9.8), | Eur. 2.9.8), |
| | | preferably 90N | preferably 180N |
| Tablet friability | | <0.5% | <0.5% |
| Disintegration time in water | | 13 | 18 |
| (seconds) | | | |

a. The film-coated, direct-compression immediate release tablet of milvexian was prepared according to provisional application US 63/483,486, which is incorporated herein by its entirety.
^{b}SDP means spray dried power comprising spray-dried amorphous solid dispersion of milvexian in HPMC-AS-MG grade polymer in a weight ratio of 3:1 (milvexian:HPMC-AS-MG).
^{c}OpadryQX 321A220063 Yellow is composed of Macrogol (PEG) polyvinyl alcohol grafted copolymer, talc, titanium dioxide, glycerol monocaprylocaprate Type I, polyvinyl alcohol and iron oxide yellow.

The SDP (spray-dried powder) in the tablets (Ex. 17, Ex. 18) is prepared as follows. A solution containing about 12.3 wt. % of milvexian P1. Acetone (equivalent to about 11.25 wt. % of milvexian free form) and about 3.75 wt. % of HPMC-AS MG (AQOAT® AS-MG, Shin-Etsu Chemical Co., Ltd. (Niigata, Japan)) in a solvent mixture containing 80/20 w/w % DCM/MeOH is prepared. The clear solution at 21° C. is spray dried using a Buchi B-290 spray dryer with a 35 Kg/hr drying gas flow-rate capacity, set of the following parameters: Atomization gas flow rate at 25 mm (301 L/hr); feed rate at 7.7 g/min; inlet/outlet temperatures at 67/44° C., condenser temperature of −19° C., spray nozzle orifice diameter of 0.7 mm, and spray nozzle cap diameter of 1.4 mm. The spray drying process proceeds for 11 min to give 11.5 g (89% yield) of wet SDP. The wet SDP is subject to drying for 24 hours in a vacuum oven (Heraeus, Model VT6130 M) at 40° C., with nitrogen flow, and a vacuum of approximately 200 mbar to give 10.7 g (83% yield) of desired dry SDP.

The SDP product is a white powder having an assay of 98.8% and a purity of 99.9% by HPLC. The PXRD diffraction pattern shows a halo pattern with no crystalline peaks indicating the product is amorphous.

### Efficacy Evaluations

The primary efficacy endpoint event is the composite of stroke and non-CNS systemic embolism. Other secondary efficacy endpoint events include: (1) the composite of CV death, MI, stroke, and non-CNS systemic embolism, (2) CV death, (3) the composite of all-cause death, MI, stroke and non-CNS systemic embolism, and (4) the composite of CV death, MI, stroke, acute limb ischemia (ALI [any unanticipated revascularization (including amputation for ischemic limb)]), and urgent hospitalization for vascular cause of ischemic nature (including thrombotic events: deep vein thrombosis (DVT) and pulmonary embolism [PE]).

All efficacy events occurring from the time of randomization through the End-of-Study contact are collected.

All primary and secondary efficacy events are assessed as time to the first occurrence of the event from randomization through the global targeted endpoint date (GTED), which is defined as the date when the projected target number of primary efficacy endpoint events has been achieved.

Exploratory efficacy events include the components collected for the primary and secondary endpoints either alone and/or in combination. Thus, exploratory objectives include:

To explore if milvexian when compared to apixaban reduces the risk of:
a) The composite of All Cause Death, MI, stroke, and ALI
b) Stroke subtypes
c) MI
d) All Cause Death
e) Fatal or disabling stroke
f) Non-CNS SE
g) Other individual components and the subtypes of a component not included above

To characterize the PK of milvexian;
To explore the exposure-response relationships for key efficacy and safety endpoints and PD endpoints; and
To collect medical resource utilization data to be incorporated in economic modeling for participants treated with milvexian compared to apixaban.

### Pharmacokinetic Evaluations

Plasma samples are collected from approximately 5,000 participants. Samples are analyzed from participants in the PK subset to determine concentration of milvexian using a validated, specific, and sensitive (eg, liquid chromatography-mass spectrometry/mass spectrometry) method.

Based on the individual plasma concentration-time data and using the actual dose and sampling times, PK parameters for exposure-response analysis of milvexian and associated variables may be derived using population PK modeling.

### Pharmacodynamic and Biomarker Evaluations

Plasma samples for PD and biomarkers assays are collected in approximately 1,000 participants in the study.

The PD assay is aPTT. Exploratory biomarkers of disease pathophysiology are proteomics and D-dimer.

### Safety Evaluations

The principal safety endpoint family includes ISTH major bleeding events and the composite of ISTH major and CRNM bleeding events. Bleeding events are assessed by other criteria (GUSTO, BARC and TIMI). All safety events occurring from the time of randomization through the EOS contact are collected. Bleeding events are assessed as time to the first occurrence of the event from randomization through 2 days after the last dose of study intervention. Overall safety and tolerability assessment will include evaluation of adverse events, clinical laboratory tests, vital signs, and physical examinations.

In particular, the safety endpoints are (1) Time to the first occurrence of ISTH major bleeding; and (2) Time to the first occurrence of the composite of ISTH major and CRNM bleeding.

### Statistical Considerations:

Event-driven; variable treatment duration, at least minimum of exposure for 13 weeks (3 months)
Non-inferiority margin: 1.37 (67% retention)
HR: 1.00 for milvexian compared to apixaban
Alpha: 2.5% (one-sided for NI); 5% (two-sided) for superiority
Relative risk: 0.75 for ISTH major bleeding
Power: 90% for ST/SE, 90% for ISTH major bleeding
Enrollment period/total study duration: 3.5/4 years
Sample Size: approximately 15,500
Number of events: 430 for ST/SE; 530 for ISTH major bleeding.

The components of the composite primary efficacy endpoint will be summarized (with estimates and 95% confidence intervals) to better understand the contribution of component endpoints on the primary efficacy endpoint. Furthermore, cumulative event rates of the primary efficacy endpoint over time will be estimated using the Kaplan-Meier method.

### Example 2. Prevention of Venous Thromboembolism after Total Knee Arthroplasty

The AXIOMATIC-TKR (Antithrombotic treatment with FXIa inhibition to Optimize Management of Acute Thromboembolic events in TKR) study was a prospective, randomized, open-label, study drug-dose blind, active-controlled, multicenter, dose-ranging, blinded endpoint study of milvexian in participants undergoing primary unilateral elective TKR surgery. The primary objective was to determine the efficacy of milvexian in preventing total venous thromboembolism (VTE) events, defined as proximal and/or distal deep vein thrombosis (DVT) (asymptomatic confirmed by venography assessment or objectively confirmed symptomatic), nonfatal pulmonary embolism, or any death, during the treatment period. The primary hypothesis testing evaluated whether the total VTE rate in the combined BID doses of milvexian was <30%. Assessment of the dose-response for the occurrence of any bleeding was a secondary objective. Participants were randomized 1:1:1:1:1:1:2 to milvexian 25 mg QD, 200 mg QD, 25 mg BID, 50 mg BID, 100 mg BID, 200 mg BID or to enoxaparin 40 mg QD subcutaneous for 10 to 14 days. After the first interim analysis, randomization into the 25 mg QD dosing group was discontinued and milvexian 50 mg QD dosing was initiated. The milvexian used in AXIOMATIC-TKR phase II trial was formulated as SDD capsule formulations in 25 mg and 100 mg strength as described in WO2020210629.

A total of 1,242 participants were randomized and included in the intention-to-treat analysis set. The median age of the study population was 68 years. The mean duration of treatment exposure was 11.7 days in the combined milvexian groups and in the enoxaparin group. Total VTE was reported in 108 (13.6%) of the 796 combined milvexian participants and in 63 (12.2%) of 518 participants assigned to a milvexian BID dose regimen, compared to 54 (21.4%) of 252 enoxaparin participants (relative risk ratio [RR] 0.64, 95% CI 0.48-0.85 for combined milvexian vs. enoxaparin, and RR 0.57, 95% CI 0.41-0.79 for milvexian BID vs. enoxaparin). There was a statistically significant reduction (P<0.0001) of total VTE for the pooled milvexian BID dose regimens compared to the prespecified target of 30%. Milvexian demonstrated statistically significant reductions in total VTE compared to enoxaparin at each of the total daily dose regimens ≥100 mg. A statistically significant dose response trend was observed for the BID regimens (P=0.0004); post-hoc analyses also demonstrated a significant dose response trend (P=0.0003) for the once-daily dose groups.

The pharmacodynamics analysis set was defined as subjects who have received at least 1 dose of study drug and at least one valid blood sample drawn for PD analysis. PD analyses were performed on the PD analysis set, defined as subjects who had at least one valid blood sample drawn for PD. Descriptive statistics (N, mean, SD, median, range, CV (%) and IQ range) were used to summarize PD response at each nominal sampling time point by treatment group. Changes from baseline were summarized when applicable. PD response outside of limit of quantitation were not to be imputed and were to be noted in the summary statistics.

The pharmacodynamic results from this study are shown in Table 2, Table 3, Table 4, Table 5 below.

TABLE 2
Summary of Activated Partial Thromboplastin Time (aPTT) and Ratio to Baseline aPTT, at Nominal
Sampling Time Day 1 Through Day 14, by Dose and Treatment Group; PD Analysis Set

| | Measured Value | | | | | | Ratio to Baseline | | | | | |
|---|---|---|---|---|---|---|---|---|---|---|---|---|
| | N | Mean | SD | Med | Min | Max | N | Mean | SD | Med | Min | Max |
| Analysis set: PD analysis set | | | | | | | | | | | | |
| Milvexian 50 mg BID | 148 | | | | | | | | | | | |
| Milvexian 100 mg BID | 149 | | | | | | | | | | | |
| ENOXAPARIN | 296 | | | | | | | | | | | |
| Activated Partial | | | | | | | | | | | | |
| Thromboplastin Time (sec) | | | | | | | | | | | | |
| Milvexian 50 mg BID | | | | | | | | | | | | |
| Baseline | 140 | 27.82 | 6.679 | 26.45 | 20 | 68.7 | | | | | | |
| Day 1 2 h | 139 | 44.12 | 10.867 | 44.5 | 21.6 | 73.7 | 135 | 1.64 | 0.44 | 1.72 | 0.5 | 2.5 |
| Day 1 4 h | 134 | 47.83 | 10.936 | 47.1 | 24.6 | 118.6 | 129 | 1.76 | 0.361 | 1.77 | 0.5 | 3.1 |
| Day 2 Predose | 138 | 54.52 | 28.131 | 50.55 | 34.8 | 358.9 | 132 | 2.02 | 0.921 | 1.99 | 0.7 | 11.3 |
| Day 4 Predose | 86 | 57.84 | 10.983 | 55.95 | 38.7 | 113.6 | 84 | 2.13 | 0.469 | 2.12 | 0.9 | 4.7 |
| Day 4 4 h | 88 | 61.72 | 13.663 | 59 | 40.6 | 124.8 | 85 | 2.28 | 0.536 | 2.23 | 0.9 | 4.7 |
| Day 10-14 | 139 | 59.26 | 26.042 | 56.4 | 24.3 | 277.4 | 133 | 2.17 | 0.89 | 2.11 | 0.5 | 10.5 |
| Milvexian 100 mg BID | | | | | | | | | | | | |
| Baseline | 142 | 27.86 | 8.85 | 26.3 | 10.7 | 93.1 | | | | | | |
| Day 1 2 h | 142 | 50.92 | 15.283 | 51.6 | 22.6 | 126.1 | 137 | 1.89 | 0.624 | 1.9 | 0.4 | 5.1 |
| Day 1 4 h | 141 | 56.19 | 22.927 | 54.1 | 22.4 | 282.4 | 136 | 2.08 | 0.959 | 2.02 | 0.5 | 11.8 |
| Day 2 Predose | 140 | 61.63 | 13.832 | 59.9 | 38.9 | 156.5 | 136 | 2.32 | 0.603 | 2.27 | 0.6 | 5.8 |
| Day 4 Predose | 87 | 67.53 | 12.679 | 66 | 39.9 | 113.4 | 83 | 2.53 | 0.605 | 2.5 | 0.9 | 5.8 |
| Day 4 4 h | 88 | 70.64 | 12.13 | 69.05 | 38.6 | 106.5 | 84 | 2.67 | 0.681 | 2.66 | 1 | 6.9 |
| Day 10-14 | 143 | 73.19 | 33.524 | 69.7 | 29.3 | 367.5 | 137 | 2.77 | 1.448 | 2.6 | 0.7 | 16.6 |
| ENOXAPARIN | | | | | | | | | | | | |
| Baseline | 283 | 27.5 | 10.508 | 26 | 19.5 | 179 | | | | | | |
| Day 1 2 h | 146 | 37.03 | 53.325 | 27.5 | 20.9 | 400 | 138 | 1.36 | 1.882 | 1.07 | 0.3 | 16.7 |
| Day 1 4 h | 148 | 29.14 | 10.133 | 27.75 | 20.8 | 120.2 | 140 | 1.08 | 0.318 | 1.07 | 0.1 | 4.2 |
| Day 2 Predose | 177 | 28.11 | 6.552 | 27.4 | 20.3 | 97.7 | 169 | 1.05 | 0.188 | 1.05 | 0.3 | 2.6 |
| Day 4 Predose | 107 | 26.03 | 2.434 | 25.8 | 20.7 | 34.8 | 100 | 0.99 | 0.159 | 0.99 | 0.1 | 1.3 |
| Day 4 4 h | 105 | 27.37 | 8.122 | 26.1 | 20 | 105.9 | 98 | 1.04 | 0.293 | 1.03 | 0.1 | 3.4 |
| Day 10-14 | 277 | 27.8 | 10.632 | 25.9 | 19.8 | 154.2 | 265 | 1.04 | 0.365 | 1 | 0.1 | 4.5 |

TABLE 03
Summary of FXI Clotting Activity (FXI:c) and % Change from Baseline, at Nominal Sampling Time
Day 1 Through Day 14, by Dose and Treatment Group; PD Analysis Set

| | Measured Value | | | | | | % Change Ratio From Baseline | | | | | |
|---|---|---|---|---|---|---|---|---|---|---|---|---|
| | N | Mean | SD | Med | Min | Max | N | Mean | SD | Med | Min | Max |
| Analysis set: PD analysis set | | | | | | | | | | | | |
| Milvexian 50 mg BID | 148 | | | | | | | | | | | |
| Milvexian 100 mg BID | 149 | | | | | | | | | | | |
| ENOXAPARIN | 296 | | | | | | | | | | | |
| Milvexian 50 mg BID | | | | | | | | | | | | |
| Baseline | 143 | 1.05 | 0.226 | 1.07 | 0.1 | 1.5 | | | | | | |
| Day 1 4 h | 143 | 0.81 | 0.2 | 0.82 | 0.1 | 1.4 | 139 | −15.8 | 84.259 | −22.64 | −91.2 | 950 |
| Day 10-14 | 138 | 0.81 | 0.266 | 0.82 | 0.2 | 1.5 | 133 | −18.47 | 55.202 | −27.66 | −76.2 | 530 |
| Milvexian 100 mg BID | | | | | | | | | | | | |
| Baseline | 145 | 1.05 | 0.224 | 1.04 | 0.1 | 1.5 | | | | | | |
| Day 1 4 h | 145 | 0.65 | 0.249 | 0.64 | 0.1 | 1.5 | 142 | −37.59 | 21.387 | −40.88 | −74.2 | 50 |
| Day 10-14 | 140 | 0.44 | 0.224 | 0.37 | 0.1 | 1.5 | 137 | −56.89 | 24.129 | −64.34 | −88.8 | 47.1 |
| ENOXAPARIN | | | | | | | | | | | | |
| Baseline | 97 | 1.16 | 0.203 | 1.16 | 0.6 | 1.5 | | | | | | |
| Day 1 4 h | 14 | 1.06 | 0.195 | 1.07 | 0.7 | 1.4 | 14 | −7.24 | 8.112 | −7.24 | −18.6 | 4.6 |
| Day 10-14 | 10 | 1.36 | 0.11 | 1.39 | 1.2 | 1.5 | 9 | 36.64 | 27.762 | 37.21 | −2.9 | 91.8 |

Key: SD = standard deviation.
Note:
N for measured value is the number of subjects with a non-missing value for the parameter at the specified time point. N for percent change from baseline is the number of subjects with non-missing values at both baseline and the postbaseline time point.

TABLE 4
Summary of Thrombin Generation Assay (TGA) Parameter: Peak Height Ratio to Baseline at Nominal
Sampling Time Day 1 Through Day 14, by Dose and Treatment Group; PD Analysis Set

| | Measured Value | | | | | | Ratio to Baseline | | | | | |
|---|---|---|---|---|---|---|---|---|---|---|---|---|
| | N | Mean | SD | Med | Min | Max | N | Mean | SD | Med | Min | Ma |
| Analysis set: PD analysis set | | | | | | | | | | | | |
| Milvexian 50 mg BID | 148 | | | | | | | | | | | |
| Milvexian 100 mg BID | 149 | | | | | | | | | | | |
| ENOXAPARIN | 296 | | | | | | | | | | | |
| ETP Peak Height | | | | | | | | | | | | |
| (nmol/L) | | | | | | | | | | | | |
| Milvexian 50 mg BID | | | | | | | | | | | | |
| Baseline | 136 | 226.99 | 79.172 | 208.71 | 0 | 539.8 | | | | | | |
| Day 1 2 h | 139 | 136.17 | 85.036 | 129.86 | 0 | 542.2 | 131 | 0.84 | 2.837 | 0.63 | 0 | 32.9 |
| Day 1 4 h | 136 | 123.8 | 73.611 | 116.88 | 0 | 371.1 | 128 | 1.14 | 6.199 | 0.56 | 0 | 70.4 |
| Day 2 Predose | 136 | 133.91 | 68.406 | 131.12 | 0 | 326.9 | 128 | 1.64 | 11.838 | 0.62 | 0 | 134.5 |
| Day 4 Predose | 85 | 106.19 | 67.273 | 92.64 | 0 | 337.2 | 80 | 1.78 | 8.355 | 0.46 | 0 | 63.5 |
| Day 4 4 h | 86 | 92.36 | 70.283 | 80.31 | 0 | 354.1 | 82 | 1.75 | 9.154 | 0.39 | 0 | 75.7 |
| Day 10-14 | 136 | 97.53 | 76.57 | 81.88 | 0 | 376.6 | 128 | 0.96 | 4.235 | 0.4 | 0 | 41.1 |
| Milvexian 100 mg BID | | | | | | | | | | | | |
| Baseline | 139 | 226.32 | 85.671 | 208.99 | 0 | 627.6 | | | | | | |
| Day 1 2 h | 140 | 109.97 | 80.255 | 83.74 | 0 | 383.9 | 133 | 3.38 | 33.099 | 0.35 | 0 | 382.2 |
| Day 1 4 h | 136 | 100.31 | 68.142 | 90.09 | 0 | 333.7 | 129 | 2.52 | 23.357 | 0.4 | 0 | 265.7 |
| Day 2 Predose | 139 | 99.64 | 67.58 | 89.19 | 0 | 362.9 | 132 | 1.77 | 15.265 | 0.39 | 0 | 175.8 |
| Day 4 Predose | 87 | 70.96 | 58.873 | 63.85 | 0 | 285 | 81 | 0.32 | 0.289 | 0.29 | 0 | 1.4 |
| Day 4 4 h | 86 | 55.94 | 54.304 | 47.34 | 0 | 258.1 | 81 | 0.24 | 0.235 | 0.18 | 0 | 1.1 |
| Day 10-14 | 140 | 57.6 | 57.165 | 37.89 | 0 | 281.2 | 132 | 0.27 | 0.274 | 0.17 | 0 | 1.8 |
| ENOXAPARIN | | | | | | | | | | | | |
| Baseline | 271 | 214.6 | 83.91 | 202.17 | 0 | 511.9 | | | | | | |
| Day 1 2 h | 143 | 171.15 | 94.909 | 158.05 | 0 | 437.8 | 129 | 1.58 | 9.418 | 0.77 | 0 | 107.7 |
| Day 1 4 h | 153 | 183.54 | 101.228 | 160.49 | 0 | 657.4 | 139 | 1.71 | 10.848 | 0.78 | 0 | 128.6 |
| Day 2 Predose | 172 | 232.14 | 90.677 | 214.37 | 0 | 582 | 155 | 2.83 | 22.001 | 1.02 | 0 | 274.9 |
| Day 4 Predose | 106 | 250.47 | 107.259 | 227.87 | 0 | 602.9 | 95 | 3.73 | 24.922 | 1.11 | 0 | 244 |
| Day 4 4 h | 105 | 210.21 | 96.606 | 196.02 | 20.5 | 530.3 | 95 | 3.2 | 21.371 | 1 | 0.1 | 209.3 |
| Day 10-14 | 270 | 213.56 | 129.563 | 201.82 | 0 | 616.6 | 253 | 1.98 | 15.467 | 0.98 | 0 | 246.8 |

Key: SD = standard deviation.
Note:
N for measured value is the number of subjects with a non-missing value for the parameter at the specified time point. N for ratio to baseline is the number of subjects with non-missing values at both baseline and the postbaseline time point.

TABLE 5
Summary of Thrombin Generation Assay (TGA) Parameter: Lag time, and Ratio to Baseline at Nominal
Sampling Time Day 1 Through Day 14, by Dose and Treatment Group; PD Analysis Set

| | Measured Value | | | | | | Ratio to Baseline | | | | | |
|---|---|---|---|---|---|---|---|---|---|---|---|---|
| | N | Mean | SD | Med | Min | Max | N | Mean | SD | Med | Min | Max |
| Analysis set: PD analysis set | | | | | | | | | | | | |
| Milvexian 50 mg BID | 148 | | | | | | | | | | | |
| Milvexian 100 mg BID | 149 | | | | | | | | | | | |
| ENOXAPARIN | 296 | | | | | | | | | | | |
| ETP Lag Time (min) | | | | | | | | | | | | |
| Milvexian 50 mg BID | | | | | | | | | | | | |
| Baseline | 136 | 7.2 | 12.435 | 5.44 | 1.3 | 120 | | | | | | |
| Day 1 2 h | 139 | 15.96 | 18.595 | 11.67 | 1.3 | 120 | 132 | 2.63 | 2.533 | 2.26 | 0.1 | 22.2 |
| Day 1 4 h | 136 | 16.82 | 16.362 | 13.22 | 3.4 | 120 | 129 | 2.92 | 2.656 | 2.46 | 0.1 | 25.1 |
| Day 2 Predose | 136 | 17.64 | 19.756 | 12.78 | 2.3 | 120 | 129 | 3.11 | 3.151 | 2.33 | 0.1 | 22.2 |
| Day 4 Predose | 85 | 23.32 | 23.724 | 16.22 | 3.4 | 120 | 81 | 3.93 | 3.866 | 3.04 | 0.1 | 27.7 |
| Day 4 4 h | 86 | 27.12 | 24.18 | 18.62 | 5.6 | 120 | 83 | 4.58 | 3.581 | 3.49 | 0.1 | 20.4 |
| Day 10-14 | 136 | 27.19 | 27.038 | 18.28 | 3 | 120 | 129 | 4.17 | 3.366 | 3.27 | 0.2 | 23.2 |
| Milvexian 100 mg BID | | | | | | | | | | | | |
| Baseline | 139 | 7.8 | 14.189 | 5.44 | 2.7 | 120 | | | | | | |
| Day 1 2 h | 140 | 20.81 | 21.312 | 14.45 | 2.8 | 120 | 134 | 3.48 | 3.167 | 2.78 | 0.2 | 20.8 |
| Day 1 4 h | 136 | 23.44 | 24.082 | 15.52 | 4.4 | 120 | 130 | 3.79 | 3.234 | 2.93 | 0.4 | 20 |
| Day 2 Predose | 139 | 23.71 | 23.561 | 16.78 | 2.6 | 120 | 133 | 3.95 | 3.448 | 3.06 | 0.6 | 21.2 |
| Day 4 Predose | 87 | 35.22 | 32.86 | 22.89 | 5.2 | 120 | 82 | 6.04 | 5.312 | 4.33 | 1 | 27.3 |
| Day 4 4 h | 86 | 39.24 | 34.166 | 27.11 | 8.2 | 120 | 81 | 6.8 | 5.12 | 4.9 | 1.9 | 24.5 |
| Day 10-14 | 140 | 37.89 | 32.398 | 25.59 | 5.4 | 120 | 134 | 6.33 | 5.209 | 4.66 | 0.2 | 28.4 |
| ENOXAPARIN | | | | | | | | | | | | |
| Baseline | 271 | 7.03 | 7.237 | 6.11 | 1.7 | 120 | | | | | | |
| Day 1 2 h | 143 | 10.81 | 18.976 | 7.02 | 1.6 | 120 | 130 | 1.94 | 3.592 | 1.27 | 0.1 | 29.2 |
| Day 1 4 h | 153 | 8.39 | 9.72 | 6.83 | 2 | 120 | 140 | 1.37 | 0.735 | 1.26 | 0.2 | 8.4 |
| Day 2 Predose | 172 | 6.98 | 9.15 | 5.7 | 2 | 120 | 156 | 1.18 | 1.131 | 1.05 | 0 | 14.4 |
| Day 4 Predose | 106 | 7.44 | 11.195 | 5.96 | 2.4 | 120 | 95 | 1.48 | 2.972 | 1.11 | 0.4 | 29.9 |
| Day 4 4 h | 105 | 8.16 | 4.554 | 7.44 | 2.4 | 40.2 | 95 | 1.46 | 0.555 | 1.36 | 0.7 | 4.2 |
| Day 10-14 | 270 | 11.18 | 13.136 | 8.11 | 3.3 | 120 | 253 | 1.7 | 1.943 | 1.31 | 0.4 | 25.7 |

Key: SD = standard deviation.
Note:
N for measured value is the number of subjects with a non-missing value for the parameter at the specified time point. N for ratio to from baseline is the number of subjects with non-missing values at both baseline and the postbaseline time point.

### Example 3. Prevention of New Ischemic Stroke or New Covert Brain Infarction Following Acute Ischemic Stroke or Transient Ischemic Attack

The Phase 2 AXIOMATIC-SSP study enrolled participants ≥40 years of age with non-lacunar, non-cardioembolic ischemic stroke (NIHSS score ≤7) or TIA within 48 hours of sign or symptom onset. Qualifying strokes were symptomatic non-lacunar acute brain infarcts visible on neuroimaging (CT or MRI) with a National Institutes of Health Stroke Scale [NIHSS] score of ≤5 and, after safety review, the NIHSS score was extended to ≤7. Qualifying TIAs were acute onset neurologic symptoms attributable to focal brain ischaemia causing motor deficits or an ABCD² score ≥6 with complete resolution of the deficit and no brain infarction on neuroimaging. All participants had imaging evidence of visible intracranial or extracranial atherosclerotic plaque, ulceration, or thrombus of any degree (but not complete occlusion) in a feeding artery and a premorbid modified Rankin score (mRS) of ≤3.

Excluded were patients with: a history of haemorrhage in the brain or spinal cord or a condition that would exclude anticoagulant therapy; SARS-CoV-2 infection within four weeks prior to screening; imaging evidence of large vessel dissection; intracranial tumour (other than meningioma) or arteriovenous malformation that could explain the qualifying symptoms; estimated glomerular filtration rate of <15 mL/min per 1·73 m²; clinically meaningful liver disease (aspartate aminotransferase or alanine aminotransferase >3×upper limit of normal); treatment with strong inducers or inhibitors of both P-glycoprotein and cytochrome P450 isoenzyme 3A4 in the seven days before randomisation or planned ongoing treatment during the study; planned use of anticoagulants (except heparins for maintaining patency of indwelling catheters); or a requirement for dual antiplatelet therapy beyond 21 days.

Eligible participants were randomized to receive 1 of 5 doses of double-blind milvexian (25, 50, 100, 200 mg twice daily, 25 mg once daily) or placebo. The milvexian used in AXIOMATIC-SSP phase II trial was formulated as SDD capsule formulations in 25 mg and 100 mg strength as described in WO2020210629.

All participants received background treatment with open-label aspirin and clopidogrel (loading dose of 300-600 mg, followed by 75 mg/day) for 21 days, followed by open-label aspirin from Day 22 to 90.

The primary efficacy outcome was to assess dose response for the composite of new clinical ischemic stroke during treatment and new covert brain infarction detected by comparing MRI brain scans at baseline and day 90 in a modified Intention to treat (ITT) population (those participants with Day 90 MRI). A total of 2,366 participants were randomized and completed study participation.

There was no significant dose-response across milvexian doses ranging from 25 mg QD to 200 mg BID for the primary composite efficacy endpoint of symptomatic ischaemic stroke and incident covert brain infarction. In a pre-specified secondary analysis, milvexian was associated with numerically fewer symptomatic ischaemic strokes at all doses except 200 mg BID Doses from 25 to 100 mg twice daily showed similar numerical relative risk reduction at approximately 30% versus placebo. A similar trend was observed within 21 days, the intended treatment period with dual antiplatelet therapy (DAPT). Milvexian had no effect compared to placebo on incident covert brain infarcts, which contributed 66% of primary outcome events.

There was no significant dose-response for the main composite safety endpoint of major bleeding, the incidence of which was low (1-2%) in the trial population. The incidence of major bleeding (main safety endpoint) was low overall (0.6% to 1.6%). There was no increase in intracranial haemorrhage or symptomatic haemorrhagic transformation of brain infarction (modified BARC type 3c) with milvexian versus placebo, and there was no fatal bleeding (BARC type 5) in the study. The rate of major bleeding for milvexian at 25 mg once daily and twice daily doses was the same as for placebo. Moderate increases in major bleeding (BARC Type 3/5) were seen in participants treated with milvexian at doses 50 mg twice daily and above. All milvexian doses were associated with increases in ISTH major and CRNM bleeding (mainly driven by CRNM), as well as all bleeding. However, there was no apparent dose response for the bleeding. The overall AE and SAE profile for milvexian appeared to be similar to placebo, with the exception of the 200 mg twice daily dose. The milvexian 200 mg twice daily dose had the highest rate of study drug discontinuations for adverse events and a higher rate of renal serious and non-serious adverse events leading to study drug discontinuation.

The observed primary composite outcome event rate of 17% in the placebo group was consistent with the pre-study estimated rate of 15% in the placebo group that underpinned the sample size calculations, and the observed 35% (95% CI: −25, 66%) reduction in the relative risk of symptomatic ischaemic stroke with milvexian 100 mg BID was consistent with the pre-study estimated 32% reduction in the relative risk of the composite primary efficacy endpoint with milvexian 100 mg BID.

### Example 4. Bioavailability Study of 25 mg and 100 mg Film Coated DC Tablet Against 25 Mg and 100 mg Comparative SDP Oral Capsule in Healthy Participants and Results

The first Phase 1 trial is an open-label, randomized, crossover study to evaluate the relative oral bioavailability, pharmacokinetics, and food effect after single dose (for Part 1, Part 3, and Part 4) or multiple-dose (for Part 2). Part 1 of this first Phase 1 study is to evaluate the relative bioavailability and food effect of a single dose of 200 mg milvexian administered as film coated DC tablet compared with 100 mg comparative SDP oral capsule under fasting and fed conditions. Part 2 of this first Phase 1 study is to characterize the pharmacokinetic (PK) of multiple twice daily administered doses for 5 days of milvexian administered as 200 mg milvexian administered as film coated DC tablet and comparative SDP oral capsules at 25 mg or 200 mg. The 25 mg and 100 mg SDP granule capsule formulations (see Table 6 below) are described in WO 2020210629 of which the capsule comprises MCC and lactose anhydrous DC in a weight ratio of 1:1 binder (MCC) to filler (lactose anhydrous). The composition and physical properties for the 25 mg and 100 mg film coated DC tablets are provided in the Table 1 above.

Blood samples were drawn at predetermined time points following drug administration as specified in the clinical study protocols. Concentration of the samples are measured using a validated analytical method (Liquid Chromatography with Tandem Mass Spectroscopy). Individual subject pharmacokinetic parameters (e.g. Cmax, AUCₗₐₛₜ, and AUC_{∞}) are derived by non-compartmental methods using Phoenix™ WinNonlin® (version 8.1, Pharsight, A Certara™ Company, L.P., Princeton, NJ, USA) software from the time-concentration profiles

TABLE 6
SDP Capsule Formulation Composition of 100 mg and 25 mg

| | 100 mg SDP | 25 mg SDP |
|---|---|---|
| Component | capsule | capsule |
| Intragranular | | |
| SDP of 75% Compound | 133.3 | 33.33 |
| (I): HPMCAS* | | |
| Microcrystalline cellulose | 167.7 | 41.925 |
| (MCC) | | |
| Lactose anhydrous DC | 167.7 | 41.925 |
| Weight ratio of MCC to lactose | 1:1 | 1:1 |
| anhydrous | | |
| Croscarmellose sodium | 12.5 | 3.125 |
| Colloidal silicon dioxide | 2.50 | 0.625 |
| Magnesium stearate | 1.25 | 0.3125 |
| Total Intragranular | 485 | 121 |
| Extragranular | | |
| Croscarmellose sodium | 12.5 | 3.125 |
| Magnesium stearate | 2.5 | 0.625 |
| Total | 500 | 125 |

*SDP prepared according to the composition and method described in WO 2020210629

The treatment regimens for Part 1 and Part 2 are summarized in Table 7 below.

TABLE 7
Treatment Regimen used in Part 1 and Part 2

| Treatment | Formulation | Dose | Food |
|---|---|---|---|
| A (Part 1A) | 100 mg film-coated DC* | 200 mg (2 × 100 mg) | Fasted |
| | oral tablet | | |
| C (Part 1A) | 100 mg SDP oral capsule | 200 mg (2 × 100 mg) | Fasted |
| D (Part 1B) | 100 mg film-coated DC* | 200 mg (2 × 100 mg) | Fed |
| | oral tablet | | |
| F (Part 1B) | 100 mg SDP oral capsule | 200 mg (2 × 100 mg) | Fed |
| G (Part 2A) | 100 mg film-coated DC* | 200 mg (2 × 100 mg) | BID for |
| | oral tablet | | 5 days |
| H (Part 2A) | 100 mg SDP oral capsule | 200 mg (2 × 100 mg) | BID for |
| | | | 5 days |
| I (Part 2B) | 25 mg film-coated DC* | 25 mg (1 × 25 mg) | BID for |
| | oral tablet | | 5 days |
| J (Part 2B) | 25 mg SDP oral capsule | 25 mg (1 × 25 mg) | BID for |
| | | | 5 days |

*DC: direct compression
**RC: roller compaction

The absolute Bioavailability for SDP capsule comparative formulation Compara. Ex. 1 is 52% for fasted and 72% for fed conditions for a 200 mg dose.

In Part 1 of single dose administration regimen, 100 mg film-coated DC tablet shows about 9.0% to about 11% as compared to the oral SDP capsule at 200 mg dose. 100 mg film-coated DC* oral tablet has lower food effects. To achieve better patient compliance, it is preferred to have milvexian being administered with or without food. A drug formulation with small food effects provide better patient compliance.

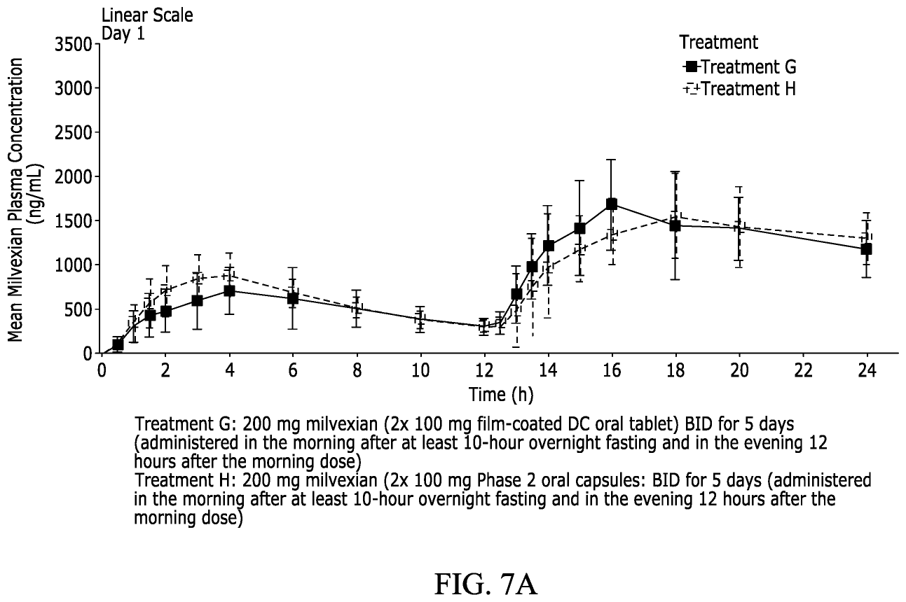

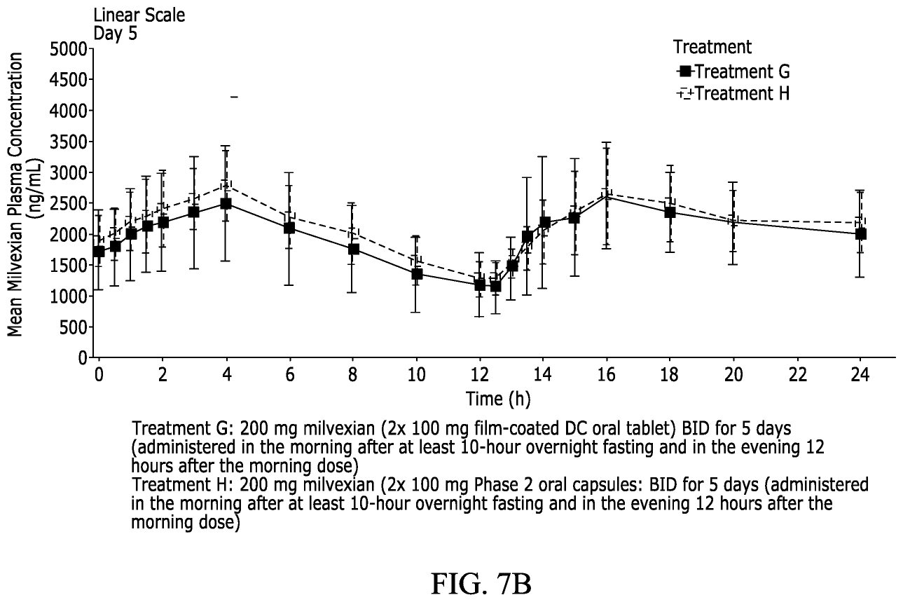

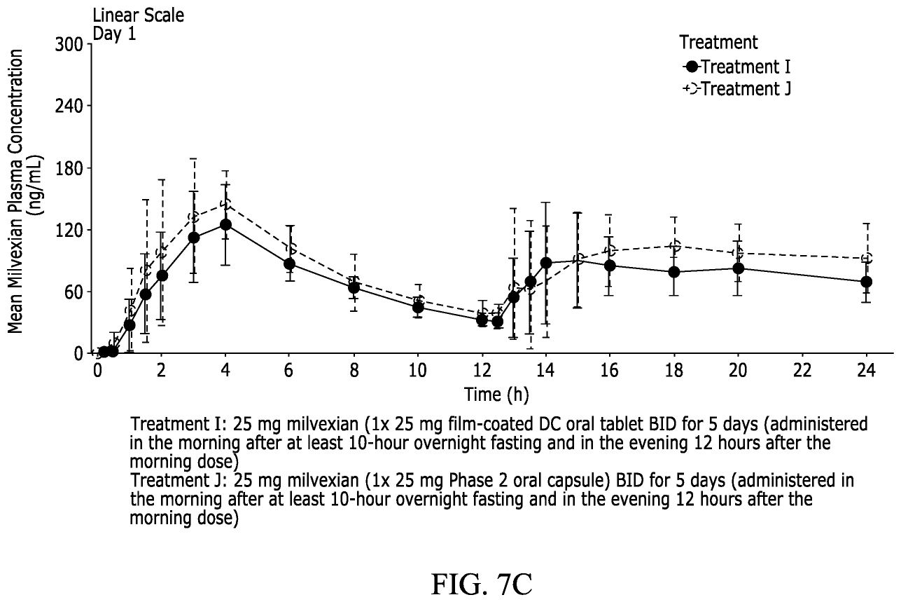

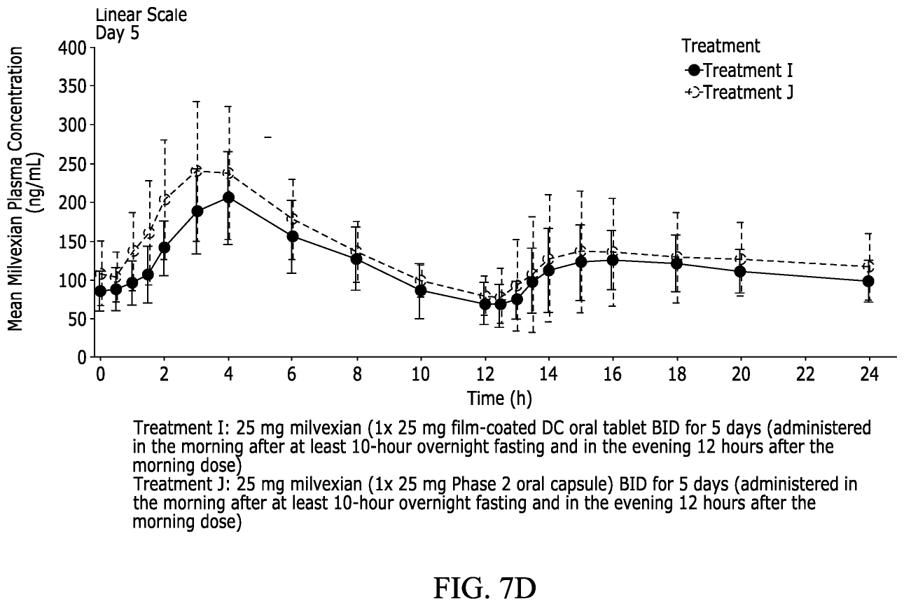

In Part 2 of multiple dose BID administration regimen, at 2×100 mg film-coated DC tablet shows about 5-7% lower bioavailability, as compared to the 2×100 mg SDP oral capsule. The 25 mg of the film-coated DC tablet (DC tablet) shows about 11-13% lower bioavailability, as compared to the 25 mg of SDP oral capsule) (See FIGS. 7A-7D for milvexian dosing curves as a function of time after BID administration).

In a second Phase 1 trial, Part 1 was an open-label, randomized, 3-way crossover study in healthy participants to evaluate the relative oral bioavailability, pharmacokinetics, and food effect of a single oral dose of 200 mg milvexian as 2×100 mg SDP DC tablet compared to 200 mg of 2×100 mg SDP granule capsule under fasting conditions and to assess the effect of food on the bioavailability of milvexian after a single dose of 200 mg milvexian as 2×100 mg SDP DC tablet. Part 2 was an open-label, randomized, 2-way crossover Study in healthy participants to evaluate the PK and relative bioavailability of a single oral dose of 50 mg milvexian as 2×25 mg SDP tablet compared to 50 mg milvexian as 2×25 mg SDP granule capsule in healthy participants under fasting conditions.

In the pooled analysis of PK (AUC_{inf} and Cₘₐₓ) data generated under the two phase 1 trials under fed and fasting conditions for 2×100 mg and 1×25 mg SDP DC tablets and 25 mg and 100 mg SDP granule capsule formulations, the results demonstrated that the tablet exhibited smaller inter-participant PK variability in healthy participants than those of capsule.

The results of clinical studies demonstrated that, for tablets with similar dissolution rates, AUC_{∞} (also known as AUC_{inf}) of the 2×100 mg film-coated DC* oral tablet coated tablet relative to the 2×100 mg granule capsule formulations, met bioequivalence criteria.

### Example 5

Delayed cardiac repolarization is an undesirable side-effect of some non-antiarrhythmic drugs. Due to the potential clinical consequences of delayed cardiac repolarization, a rigorous characterization of a new pharmaceutical agent's ability to prolong QT/QTc interval was recommended.

Results from in vitro studies indicated that milvexian inhibited cardiac potassium (hERG/IKr) channel currents with weak to moderate potency at concentrations that greatly exceed unbound concentrations in the plasma of subjects treated with clinically relevant dose regimens of milvexian. The present study, which was conducted after the in vitro study results became available, evaluated the effect of milvexian on cardiac repolarization in healthy subjects, an important aspect of cardiovascular safety.

This placebo- and positive-controlled, TQT study was conducted to evaluate the effect of multiple-dose administration of milvexian on the QT/QTc interval duration and ECG morphology at therapeutic and supratherapeutic doses at steady state, in healthy adults.

| | |
|---|---|
| Objectives | Endpoints |

Primary

| | |
|---|---|
| To assess the effects of milvexian on the | QTcF, change from baseline in QTcF |
| QT/QTcF intervals in healthy participants | (ΔQTcF), placebo-corrected change from |
| when administered at therapeutic (100 mg | baseline in QTcF (ΔΔQTcF). |
| twice daily as capsule) and supratherapeutic | |
| (200 mg twice daily as solution) doses for 4 | |
| days. | |

Secondary

| | |
|---|---|
| To assess the effects of milvexian on other | HR, ΔHR, ΔΔHR, PR, ΔPR, ΔΔPR, QRS |
| ECG parameters and ECG intervals in | duration, ΔQRS duration, and ΔΔQRS |
| healthy participants when administered at | duration. |
| therapeutic (100 mg twice daily as capsule) | |
| and supratherapeutic (200 mg twice daily as | |
| solution) doses for 4 days. | |
| To assess the assay sensitivity by evaluating | QTc, baseline QTc, change from baseline in |
| the mean ΔQTc between moxifloxacin and | QTc (ΔQTc), placebo-corrected change |
| placebo (ΔΔQTc) on Day 4. | from baseline in QTc (ΔΔQTc) for QTcB |
| | and QTcP. |
| To assess the relationship between the | Characterization of the milvexian |
| systemic plasma concentrations of | concentration - ΔΔQTcF relationship. |
| milvexian and QT/QTc changes. | |

The upper bound of two-sided 90% CI for the largest difference in mean change from baseline in QTc intervals (ΔQTc; based on the primary correction method—QTcF) between milvexian and placebo (ΔΔQTc) at the therapeutic (100 mg twice daily as capsule) and supratherapeutic (200 mg twice daily as solution) doses was less than 10 milliseconds.

A total of 66 participants were enrolled in the study and randomly assigned to 1 of 4 possible treatment sequences. All 66 (100.0%) participants enrolled in the study were included in the safety and PD analysis set: 55 participants each in the milvexian 100 mg and 200 mg groups, 58 participants in the moxifloxacin group, and 57 participants in the placebo group.

Pharmacokinetic analysis was performed on the plasma concentrations of each participant. Sixty-one participants were included in the PK analysis set, i.e., all randomized participants who received at least one dose of active study intervention whose PK profiles allowed for accurate calculation of at least one PK parameter. The PK/PD analysis was based on the participants included in the PD analysis set who had at least one measurement of milvexian concentration.

All 66 (100.0%) participants enrolled in the study received at least one entire dose of study intervention.

Forty-five (68.2%) participants completed all 4 periods with study intervention as planned. Per study intervention, 52 participants received milvexian 100 mg capsule twice daily for 4 days, 54 participants received milvexian 200 mg solution twice daily for 4 days, 58 participants received a single dose of 400 mg moxifloxacin, and 56 participants received placebo for 4 days.

There were no consistent or clinically relevant changes over time in mean vital signs.

No treatment-emergent actual values of QTcF or QTcB>480 ms or changes from baseline >60 ms were observed during the study. No ECG abnormalities were reported as TEAE in the study.

Milvexian exposure (i.e., Cₘₐₓ and AUC over a dosing interval) was approximately 2- to 3-times higher on Day 4, compared with Day 1, when 100 mg was administered twice daily as a capsule (Treatment A) and 200 mg was administered twice daily as a solution (Treatment B).

### Pharmacodynamic Results:

On administration of 100 and 200 mg milvexian, the upper limits of the 2-sided 90% CIs for ΔΔQTcF over the Day 1 and Day 4 postdose measurement intervals were below the protocol specified 10-ms limit at all timepoints (i.e., highest upper limits of 5.16 ms and 4.57 ms for 100 mg and 200 mg milvexian, respectively), showing there was no effect of clinical or regulatory concern on the QT interval in accordance with the ICH E14 guideline. These results were confirmed based on the ΔΔQTcF at milvexian Tₘₐₓ, i.e., the upper limits of the 90% CI were below 10 ms at the Tₘₐₓ of milvexian after administration of 100 mg or 200 mg twice daily, from analyses that used milvexian Tₘₐₓ on Day 1 and Day 4 (combined) or milvexian Tₘₐₓ on Day 1 and Day 4 separately.

Study-specific and Bazett's correction methods also showed there was no effect of clinical or regulatory concern on the QT interval in accordance with the ICH E14 guideline. Assay sensitivity for moxifloxacin was also demonstrated when using Bazett's or the study-specific power correction methods for HR.

No treatment-emergent actual values of QTc >480 ms were observed during the study. No treatment-emergent changes from baseline of QTcF or QTcP >60 ms were observed during the study. One (1.7%) participant had a treatment-emergent change from baseline of QTcB>60 ms during the study (i.e., following moxifloxacin).

No consistent or clinically relevant changes over time were observed in HR, RR interval, PR interval, or QRS width. No clinically meaningful differences were observed between treatment groups

### Pharmacokinetics/Pharmacodynamics Results:

Based on the results of linear mixed effects modeling, no statistical significant relationship was observed between milvexian concentration and ΔΔQTcF (p=0.8454), ΔΔQTcP (p=0.7102), or ΔΔQTcB (p=0.8670). At Cₘₐₓ, no statistically significant effect of treatment (100 or 200 mg milvexian) on ΔΔQTc was observed (p>0.5).

### CONCLUSIONS

No notable study limitations were identified by the sponsor.

Administration of multiple doses of 100 and 200 mg milvexian was generally safe and well tolerated in healthy adult participants.

The upper limits of all 2-sided 90% CIs of the time-matched differences between 100 mg milvexian and placebo and between 200 mg milvexian and placebo in changes from baseline in QTcF were below 10 ms. Hence, milvexian at therapeutic (100 mg) and supratherapeutic (200 mg) doses does not show evidence of QT/QTc interval prolongation of clinical or regulatory concern, according to the ICH E14 guideline.

Assay sensitivity was demonstrated by moxifloxacin as the positive control.

Based on the results of linear mixed effects modeling, no statistical significant relationship was observed between milvexian concentration and ΔΔQTcF (p=0.8454).

1. A method for preventing one or more of stroke and non-central nervous system (CNS) systemic embolism events in a human patient with a history of atrial fibrillation, wherein the method comprises administering to the human patient a pharmaceutical composition comprising 100 mg of milvexian (or a pharmaceutically acceptable salt or solvate thereof, on a milvexian basis); wherein the pharmaceutical composition is administered twice daily.

2. A method for preventing one or more of adverse cardiovascular events in a patient with a history of atrial fibrillation, wherein each event is selected from the group consisting of cardiovascular death, myocardial infarction, stroke, and non-central nervous system (CNS) systemic embolism, wherein the method comprises administering to the human patient a pharmaceutical composition comprising 100 mg of milvexian (or a pharmaceutically acceptable salt or solvate thereof, on a milvexian basis); wherein the composition is administered twice daily.

3. The method of claim 2, wherein the adverse cardiovascular event is cardiovascular death.

4. The method of claim 1, wherein the patient has a history of atrial fibrillation, and also has concomitant coronary artery disease (CAD) and/or peripheral artery disease (PAD).

5. The method of claim 1, wherein the pharmaceutical composition comprises 100 mg of milvexian.

6. The method of claim 1, wherein the pharmaceutical composition is a solid oral pharmaceutical composition.

7. The method of claim 6, wherein the solid oral pharmaceutical composition is an immediate release tablet.

8. The method of claim 1, wherein the administration results in milvexian plasma concentrations reaching steady-state at about 3 days to 6 days.

9. The method of claim 1, wherein the pharmaceutical composition is administered without regard to the timing of food intake.

10. A method of preventing one or more of stroke and non-central nervous system (CNS) systemic embolism events in a patient with a history of atrial fibrillation, wherein the method comprises administering to the patient an immediate release film coated tablet comprising 100 mg of milvexian or a pharmaceutically acceptable salt thereof and one or more pharmaceutically acceptable excipients,
wherein the immediate release film-coated tablet is administered twice a day with or without a meal,
wherein the patient is at least 18 years old;
wherein the patient further has one or more of category (A) risk factors selected from (i) age greater than or equal to 75 years or (ii) history of ischemic stroke or silent brain infarct; or two or more of concomitant or comorbid category (B) cardiovascular risk factors selected from the group consisting of:
(i) age between 65 and 74 years;
(ii) hypertension;
(iii) diabetes mellitus;
(iv) history of coronary artery disease (CAD) (including post myocardial infarction or an acute coronary syndrome event with or without percutaneous coronary intervention (PCI)) or peripheral artery disease (PAD); and
(v) history of heart failure.

11. A method of preventing one or more adverse cardiovascular events in a patient with a history of atrial fibrillation, wherein each event is selected from the group consisting of cardiovascular death, myocardial infarction, stroke, and non-central nervous system (CNS) systemic embolism, said method comprising administering to the patient an immediate release film-coated tablet comprising 100 mg of milvexian, or a pharmaceutically acceptable salt thereof, and one or more pharmaceutically acceptable excipients,
wherein the immediate release film-coated tablet is administered twice a day with or without a meal,
wherein the patient is at least 18 years old;
wherein the patient further has one or more of category (A) risk factors selected from (i) age greater than or equal to 75 years or (ii) history of ischemic stroke or silent brain infarct; or two or more of concomitant or comorbid category (B) cardiovascular risk factors selected from the group consisting of:
(i) age between 65 and 74 years;
(ii) hypertension;
(iii) diabetes mellitus;
(iv) history of coronary artery disease (CAD) (including post myocardial infarction or an acute coronary syndrome event with or without percutaneous coronary intervention (PCI)) or peripheral artery disease (PAD); and
(v) history of heart failure.

12. The method of claim 11, wherein the adverse cardiovascular event is cardiovascular death.

13. The method of claim 10, wherein the administered milvexian has a plasma half-life of 13-16 hours.

14. The method of claim 1, wherein the administration reduces factor XI (FXI) clotting activity in the patient by about 27% to about 64% relative to the baseline FXI clotting activity.

15. The method of claim 1, wherein the administration results in a prolongation of activated partial thromboplastin time (aPTT) in the patient with a ratio to baseline of 2.1 to 2.6.

16. The method of claim 1, wherein the pharmaceutical composition is administered twice daily for at least 13 weeks.

17. The method of claim 1, wherein the pharmaceutical composition is administered once daily in the morning and once daily in the evening.

18. The method of claim 17, wherein the morning administration is made at the same time each day and the evening administration is made at the same time each day.

19. The method of claim 1, wherein the milvexian administration results in no clinically significant corrected QT (QTc) interval prolongation.
